SEQUENCE LISTING (246 sequences)

SEQ ID NO: 1 - RNA, 76 nt, synthetic construct
  misc_feature 1..76: note = Artificial sequence generated for Engineered CRISPR-Cas System sand Methods for Sensitive and Specific Diagnostics
  source 1..76: mol_type = other RNA
attgcgacacgctgaagcgctgggggcaaattgtgcaatttgcggccagttgcaagggattgagccccgtaagggg

SEQ ID NO: 2 - RNA, 56 nt, synthetic construct
  misc_feature 1..56: note = Artificial sequence generated for Engineered CRISPR-Cas System sand Methods for Sensitive and Specific Diagnostics
  source 1..56: mol_type = other RNA
attgcgacacgctgaagcgctgggggcaaattgtgcaatttgcggccagttgcaag

SEQ ID NO: 3 - RNA, 76 nt, synthetic construct; identical to SEQ ID NO: 1
  misc_feature 1..76: note = Artificial sequence generated for Engineered CRISPR-Cas System sand Methods for Sensitive and Specific Diagnostics
  source 1..76: mol_type = other RNA

SEQ ID NO: 4 - RNA, 76 nt, synthetic construct
  misc_feature 1..76: note = Artificial sequence generated for Engineered CRISPR-Cas System sand Methods for Sensitive and Specific Diagnostics
  source 1..76: mol_type = other RNA
attgcgacggccgacgttgttttgatcgcgccccactgcgttctccatgttgcaagggattgagccccgtaagggg

SEQ ID NO: 5 - RNA, 76 nt, synthetic construct
  misc_feature 1..76: note = Artificial sequence generated for Engineered CRISPR-Cas System sand Methods for Sensitive and Specific Diagnostics
  source 1..76: mol_type = other RNA
attgcgacgttgcgactacgtgatgaggaacgagaagaggcttgactggttgcaagggattgagccccgtaagggg

SEQ ID NO: 6 - RNA, 76 nt, synthetic construct
  misc_feature 1..76: note = Artificial sequence generated for Engineered CRISPR-Cas System sand Methods for Sensitive and Specific Diagnostics
  source 1..76: mol_type = other RNA
attgcgacagcagcagcaaagcaagagcagcatcaccgccattgccaggttgcaagggattgagccccgtaagggg

SEQ ID NO: 7 - RNA, 76 nt, synthetic construct
  misc_feature 1..76: note = Artificial sequence generated for Engineered CRISPR-Cas System sand Methods for Sensitive and Specific Diagnostics
  source 1..76: mol_type = other RNA
attgcgacatgctttagtggcagtacgtttttgccgaggcttcttagagttgcaagggattgagccccgtaagggg

SEQ ID NO: 8 - RNA, 76 nt, synthetic construct
  misc_feature 1..76: note = Artificial sequence generated for Engineered CRISPR-Cas System sand Methods for Sensitive and Specific Diagnostics
  source 1..76: mol_type = other RNA
attgcgacttccgaagaacgctgaagcgctgggggcaaattgtgcaatgttgcaagggattgagccccgtaagggg

SEQ ID NO: 9 - RNA, 76 nt, synthetic construct
  misc_feature 1..76: note = Artificial sequence generated for Engineered CRISPR-Cas System sand Methods for Sensitive and Specific Diagnostics
  source 1..76: mol_type = other RNA
attgcgacattcagcaaaatgacttgatctttgaaatttggatctttggttgcaagggattgagccccgtaagggg

SEQ ID NO: 10 - RNA, 76 nt, synthetic construct
  misc_feature 1..76: note = Artificial sequence generated for Engineered CRISPR-Cas System sand Methods for Sensitive and Specific Diagnostics
  source 1..76: mol_type = other RNA
attgcgaccagtttgctgtttcttctgtctctgcggtaaggcttgagtgttgcaagggattgagccccgtaagggg

SEQ ID NO: 11 - RNA, 76 nt, synthetic construct
  misc_feature 1..76: note = Artificial sequence generated for Engineered CRISPR-Cas System sand Methods for Sensitive and Specific Diagnostics
  source 1..76: mol_type = other RNA
attgcgacgtcagcactgctcatggattgttgcaattgtttggagaaagttgcaagggattgagccccgtaagggg

SEQ ID NO: 12 - RNA, 76 nt, synthetic construct
  misc_feature 1..76: note = Artificial sequence generated for Engineered CRISPR-Cas System sand Methods for Sensitive and Specific Diagnostics
  source 1..76: mol_type = other RNA
attgcgacaaaagcgaaaacgtttatatagcccatctgccttgtgtgggttgcaagggattgagccccgtaagggg

SEQ ID NO: 13 - DNA, 58 bp, synthetic construct
  misc_feature 1..58: note = Artificial sequence generated for Engineered CRISPR-Cas System sand Methods for Sensitive and Specific Diagnostics
  source 1..58: mol_type = other DNA
gataatacgactcactatagggaactgattacaaacattggccgcaaattgcacaatt

SEQ ID NO: 14 - DNA, 56 bp, synthetic construct
  misc_feature 1..56: note = Artificial sequence generated for Engineered CRISPR-Cas System sand Methods for Sensitive and Specific Diagnostics
  source 1..56: mol_type = other DNA
gcgcgacattccgaagaacgctgaagcgctgggggcaaattgtgcaatttgcggcc

SEQ ID NO: 15 - DNA, 58 bp, synthetic construct; identical to SEQ ID NO: 13
  misc_feature 1..58: note = Artificial sequence generated for Engineered CRISPR-Cas System sand Methods for Sensitive and Specific Diagnostics
  source 1..58: mol_type = other DNA

SEQ ID NO: 16 - DNA, 56 bp, synthetic construct
  misc_feature 1..56: note = Artificial sequence generated for Engineered CRISPR-Cas System sand Methods for Sensitive and Specific Diagnostics
  source 1..56: mol_type = other DNA
gcgtgacattccaaagaatgcagaggcacttggagcaaattgtgcaatttgcggcc

SEQ ID NO: 17 - DNA, 58 bp, synthetic construct
  misc_feature 1..58: note = Artificial sequence generated for Engineered CRISPR-Cas System sand Methods for Sensitive and Specific Diagnostics
  source 1..58: mol_type = other DNA
ctagagctcgataatacgactcactatagggcgtgttgttttagatttcatctaaacg

SEQ ID NO: 18 - DNA, 35 bp, synthetic construct
  misc_feature 1..35: note = Artificial sequence generated for Engineered CRISPR-Cas System sand Methods for Sensitive and Specific Diagnostics
  source 1..35: mol_type = other DNA
atcctgcaggcacactgattaaagattgctatgtg

SEQ ID NO: 19 - DNA, 18 bp, synthetic construct
  misc_feature 1..18: note = Artificial sequence generated for Engineered CRISPR-Cas System sand Methods for Sensitive and Specific Diagnostics
  source 1..18: mol_type = other DNA
gctgctgaggcttctaag

SEQ ID NO: 20 - DNA, 21 bp, synthetic construct
  misc_feature 1..21: note = Artificial sequence generated for Engineered CRISPR-Cas System sand Methods for Sensitive and Specific Diagnostics
  source 1..21: mol_type = other DNA
gcgtcaatatgcttattcagc

SEQ ID NO: 21 - DNA, 39 bp, synthetic construct
  misc_feature 1..39: note = Artificial sequence generated for Engineered CRISPR-Cas System sand Methods for Sensitive and Specific Diagnostics
  source 1..39: mol_type = other DNA
tcagcgttcttcggaatgtcgctgtgtaggtcaaccacg

SEQ ID NO: 22 - DNA, 40 bp, synthetic construct
  misc_feature 1..40: note = Artificial sequence generated for Engineered CRISPR-Cas System sand Methods for Sensitive and Specific Diagnostics
  source 1..40: mol_type = other DNA
gcggccaatgtttgtaatcagtagacgtggtccagaacaa

SEQ ID NO: 23 - DNA, 22 bp, synthetic construct
  misc_feature 1..22: note = Artificial sequence generated for Engineered CRISPR-Cas System sand Methods for Sensitive and Specific Diagnostics
  source 1..22: mol_type = other DNA
ccttgtctgattagttcctggt

SEQ ID NO: 24 - DNA, 18 bp, synthetic construct
  misc_feature 1..18: note = Artificial sequence generated for Engineered CRISPR-Cas System sand Methods for Sensitive and Specific Diagnostics
  source 1..18: mol_type = other DNA
tggcatggaagtcacacc

SEQ ID NO: 25 - DNA, 38 bp, synthetic construct
  misc_feature 1..38: note = Artificial sequence generated for Engineered CRISPR-Cas System sand Methods for Sensitive and Specific Diagnostics
  source 1..38: mol_type = other DNA
taatacgactcactatagggagacgtggtccagaacaa

SEQ ID NO: 26 - protein, 241 aa, Thermus thermophilus
MKLKKVIRIRSVLLAKTGLRIGMSRDQMAIGDLDNPVVRNPLTDEPYIPGSSLKGKLRYLLEWSLGGDYILKAKERQVYASPDPKDPVARIFGLAPENDERSLAVARERGPTRLLVRDAYLTEDAKEALERTSARGGLYTEIKQEVFIPRLGGNANPRTTERVPAGARFRVEMTYRVLDDLDEEYFGKYLLRALELLELDGLGGHISRGYGQVYFLHPERLTEDQEGWPLKERLKVEEVVL

SEQ ID NO: 27 - protein, 241 aa, synthetic construct
MKLKKVIRIRSVLLAKTGLRIGMSRDQMAIGDLANPVVRNPLTDEPYIPGSSLKGKLRYLLEWSLGGDYILKAKERQVYASPDPKDPVARIFGLAPENDERSLAVARERGPTRLLVRDAYLTEDAKEALERTSARGGLYTEIKQEVFIPRLGGNANPRTTERVPAGARFRVEMTYRVLDDLDEEYFGKYLLRALELLELDGLGGHISRGYGQVYFLHPERLTEDQEGWPLKERLKVEEVVL

SEQ ID NO: 28 - DNA, 37 bp, synthetic construct
  misc_feature 1..37: note = Artificial sequence generated for Engineered CRISPR-Cas System sand Methods for Sensitive and Specific Diagnostics
  source 1..37: mol_type = other DNA
taatacgactcactatagggagacgtgtccagaacaa

SEQ ID NO: 29 - DNA, 24 bp, synthetic construct
  misc_feature 1..24: note = Artificial sequence generated for Engineered CRISPR-Cas System sand Methods for Sensitive and Specific Diagnostics
  source 1..24: mol_type = other DNA
accaaccaactttcgatctcttgt

SEQ ID NO: 30 - DNA, 25 bp, synthetic construct
  misc_feature 1..25: note = Artificial sequence generated for Engineered CRISPR-Cas System sand Methods for Sensitive and Specific Diagnostics
  source 1..25: mol_type = other DNA
catctttaagatgttgacgtgcctc

SEQ ID NO: 31 - DNA, 22 bp, synthetic construct
  misc_feature 1..22: note = Artificial sequence generated for Engineered CRISPR-Cas System sand Methods for Sensitive and Specific Diagnostics
  source 1..22: mol_type = other DNA
cggtaataaaggagctggtggc

SEQ ID NO: 32 - DNA, 24 bp, synthetic construct
  misc_feature 1..24: note = Artificial sequence generated for Engineered CRISPR-Cas System sand Methods for Sensitive and Specific Diagnostics
  source 1..24: mol_type = other DNA
aaggtgtctgcaattcatagctct

SEQ ID NO: 33 - DNA, 22 bp, synthetic construct
  misc_feature 1..22: note = Artificial sequence generated for Engineered CRISPR-Cas System sand Methods for Sensitive and Specific Diagnostics
  source 1..22: mol_type = other DNA
tggtgaaacttcatggcagacg

SEQ ID NO: 34 - DNA, 28 bp, synthetic construct
  misc_feature 1..28: note = Artificial sequence generated for Engineered CRISPR-Cas System sand Methods for Sensitive and Specific Diagnostics
  source 1..28: mol_type = other DNA
attgatgttgactttctctttttggagt

SEQ ID NO: 35 - DNA, 22 bp, synthetic construct
  misc_feature 1..22: note = Artificial sequence generated for Engineered CRISPR-Cas System sand Methods for Sensitive and Specific Diagnostics
  source 1..22: mol_type = other DNA
atcagaggctgctcgtgttgta

SEQ ID NO: 36 - DNA, 22 bp, synthetic construct
  misc_feature 1..22: note = Artificial sequence generated for Engineered CRISPR-Cas System sand Methods for Sensitive and Specific Diagnostics
  source 1..22: mol_type = other DNA
catttgcatcagaggctgctcg

SEQ ID NO: 37 - DNA, 22 bp, synthetic construct
  misc_feature 1..22: note = Artificial sequence generated for Engineered CRISPR-Cas System sand Methods for Sensitive and Specific Diagnostics
  source 1..22: mol_type = other DNA
tgcacaggtgacaatttgtcca

SEQ ID NO: 38 - DNA, 22 bp, synthetic construct
  misc_feature 1..22: note = Artificial sequence generated for Engineered CRISPR-Cas System sand Methods for Sensitive and Specific Diagnostics
  source 1..22: mol_type = other DNA
aggtgacaatttgtccaccgac

SEQ ID NO: 39 - DNA, 24 bp, synthetic construct
  misc_feature 1..24: note = Artificial sequence generated for Engineered CRISPR-Cas System sand Methods for Sensitive and Specific Diagnostics
  source 1..24: mol_type = other DNA
tcccacagaagtgttaacagagga

SEQ ID NO: 40 - DNA, 24 bp, synthetic construct
  misc_feature 1..24: note = Artificial sequence generated for Engineered CRISPR-Cas System sand Methods for Sensitive and Specific Diagnostics
  source 1..24: mol_type = other DNA
ttcccacagaagtgttaacagagg

SEQ ID NO: 41 - DNA, 22 bp, synthetic construct
  misc_feature 1..22: note = Artificial sequence generated for Engineered CRISPR-Cas System sand Methods for Sensitive and Specific Diagnostics
  source 1..22: mol_type = other DNA
atgacagcatctgccacaacac

SEQ ID NO: 42 - DNA, 22 bp, synthetic construct
  misc_feature 1..22: note = Artificial sequence generated for Engineered CRISPR-Cas System sand Methods for Sensitive and Specific Diagnostics
  source 1..22: mol_type = other DNA
gacagcatctgccacaacacag

SEQ ID NO: 43 - DNA, 22 bp, synthetic construct
  misc_feature 1..22: note = Artificial sequence generated for Engineered CRISPR-Cas System sand Methods for Sensitive and Specific Diagnostics
  source 1..22: mol_type = other DNA
ggaatttggtgccacttctgct

SEQ ID NO: 44 - DNA, 24 bp, synthetic construct
  misc_feature 1..24: note = Artificial sequence generated for Engineered CRISPR-Cas System sand Methods for Sensitive and Specific Diagnostics
  source 1..24: mol_type = other DNA
tcatcagattcaacttgcatggca

SEQ ID NO: 45 - DNA, 24 bp, synthetic construct
  misc_feature 1..24: note = Artificial sequence generated for Engineered CRISPR-Cas System sand Methods for Sensitive and Specific Diagnostics
  source 1..24: mol_type = other DNA
tcgcacaaatgtctacttagctgt

SEQ ID NO: 46 - DNA, 22 bp, synthetic construct
  misc_feature 1..22: note = Artificial sequence generated for Engineered CRISPR-Cas System sand Methods for Sensitive and Specific Diagnostics
  source 1..22: mol_type = other DNA
accacagcagttaaaacaccct

SEQ ID NO: 47 - DNA, 27 bp, synthetic construct
  misc_feature 1..27: note = Artificial sequence generated for Engineered CRISPR-Cas System sand Methods for Sensitive and Specific Diagnostics
  source 1..27: mol_type = other DNA
acagtgcttaaaaagtgtaaaagtgcc

SEQ ID NO: 48 - DNA, 26 bp, synthetic construct
  source 1..26: mol_type = other DNA
  misc_feature 1..26: note = Artificial sequence generated for Engineered CRISPR-Cas Systems and Methods for Sensitive and Specific Diagnostics
agtgcttaaaaagtgtaaaagtgcct

SEQ ID NO: 49 - DNA, 22 bp, synthetic construct
  misc_feature 1..22: note = Artificial sequence generated for Engineered CRISPR-Cas System sand Methods for Sensitive and Specific Diagnostics
  source 1..22: mol_type = other DNA
aacagaaactgtagctggcact

SEQ ID NO: 50 - DNA, 23 bp, synthetic construct
  misc_feature 1..23: note = Artificial sequence generated for Engineered CRISPR-Cas System sand Methods for Sensitive and Specific Diagnostics
  source 1..23: mol_type = other DNA
actgtagctggcactttgagaga

SEQ ID NO: 51 - DNA, 27 bp, synthetic construct
  misc_feature 1..27: note = Artificial sequence generated for Engineered CRISPR-Cas System sand Methods for Sensitive and Specific Diagnostics
  source 1..27: mol_type = other DNA
cttctttctttgagagaagtgaggact

SEQ ID NO: 52 - DNA, 25 bp, synthetic construct
  misc_feature 1..25: note = Artificial sequence generated for Engineered CRISPR-Cas System sand Methods for Sensitive and Specific Diagnostics
  source 1..25: mol_type = other DNA
tttgttggagtgttaacaatgcagt

SEQ ID NO: 53 - DNA, 23 bp, synthetic construct
  misc_feature 1..23: note = Artificial sequence generated for Engineered CRISPR-Cas System sand Methods for Sensitive and Specific Diagnostics
  source 1..23: mol_type = other DNA
gctgttatgtacatgggcacact

SEQ ID NO: 54 - DNA, 25 bp, synthetic construct
  misc_feature 1..25: note = Artificial sequence generated for Engineered CRISPR-Cas System sand Methods for Sensitive and Specific Diagnostics
  source 1..25: mol_type = other DNA
tgtccaacttagggtcaatttctgt

SEQ ID NO: 55 - DNA, 29 bp, synthetic construct
  misc_feature 1..29: note = Artificial sequence generated for Engineered CRISPR-Cas System sand Methods for Sensitive and Specific Diagnostics
  source 1..29: mol_type = other DNA
tggctattgattataaacactacacaccc

SEQ ID NO: 56 - DNA, 29 bp, synthetic construct
  misc_feature 1..29: note = Artificial sequence generated for Engineered CRISPR-Cas System sand Methods for Sensitive and Specific Diagnostics
  source 1..29: mol_type = other DNA
ggctattgattataaacactacacaccct

SEQ ID NO: 57 - DNA, 22 bp, synthetic construct
  misc_feature 1..22: note = Artificial sequence generated for Engineered CRISPR-Cas System sand Methods for Sensitive and Specific Diagnostics
  source 1..22: mol_type = other DNA
tagatctgtgtggccaacctct

SEQ ID NO: 58 - DNA, 22 bp, synthetic construct
  misc_feature 1..22: note = Artificial sequence generated for Engineered CRISPR-Cas System sand Methods for Sensitive and Specific Diagnostics
  source 1..22: mol_type = other DNA
gatctgtgtggccaacctcttc

SEQ ID NO: 59 - DNA, 27 bp, synthetic construct
  misc_feature 1..27: note = Artificial sequence generated for Engineered CRISPR-Cas System sand Methods for Sensitive and Specific Diagnostics
  source 1..27: mol_type = other DNA
acaactactaacatagttacacggtgt

SEQ ID NO: 60 - DNA, 25 bp, synthetic construct
  misc_feature 1..25: note = Artificial sequence generated for Engineered CRISPR-Cas System sand Methods for Sensitive and Specific Diagnostics
  source 1..25: mol_type = other DNA
accagtacagtaggttgcaatagtg

SEQ ID NO: 61 - DNA, 27 bp, synthetic construct
  misc_feature 1..27: note = Artificial sequence generated for Engineered CRISPR-Cas System sand Methods for Sensitive and Specific Diagnostics
  source 1..27: mol_type = other DNA
gcaattgtttttcagctattttgcagt

SEQ ID NO: 62 - DNA, 23 bp, synthetic construct
  misc_feature 1..23: note = Artificial sequence generated for Engineered CRISPR-Cas System sand Methods for Sensitive and Specific Diagnostics
  source 1..23: mol_type = other DNA
actgtagtgacaagtctctcgca

SEQ ID NO: 63 - DNA, 25 bp, synthetic construct
  misc_feature 1..25: note = Artificial sequence generated for Engineered CRISPR-Cas System sand Methods for Sensitive and Specific Diagnostics
  source 1..25: mol_type = other DNA
actacagtcagcttatgtgtcaacc

SEQ ID NO: 64 - DNA, 22 bp, synthetic construct
  misc_feature 1..22: note = Artificial sequence generated for Engineered CRISPR-Cas System sand Methods for Sensitive and Specific Diagnostics
  source 1..22: mol_type = other DNA
aatacaagcaccaaggtcacgg

SEQ ID NO: 65 - DNA, 24 bp, synthetic construct
  misc_feature 1..24: note = Artificial sequence generated for Engineered CRISPR-Cas System sand Methods for Sensitive and Specific Diagnostics
  source 1..24: mol_type = other DNA
acttgtgttcctttttgttgctgc

SEQ ID NO: 66 - DNA, 29 bp, synthetic construct
  misc_feature 1..29: note = Artificial sequence generated for Engineered CRISPR-Cas System sand Methods for Sensitive and Specific Diagnostics
  source 1..29: mol_type = other DNA
agtgtactctataagttttgatggtgtgt

SEQ ID NO: 67 - DNA, 22 bp, synthetic construct
  misc_feature 1..22: note = Artificial sequence generated for Engineered CRISPR-Cas System sand Methods for Sensitive and Specific Diagnostics
  source 1..22: mol_type = other DNA
ttctgagtactgtaggcacggc

SEQ ID NO: 68 - DNA, 28 bp, synthetic construct
  misc_feature 1..28: note = Artificial sequence generated for Engineered CRISPR-Cas System sand Methods for Sensitive and Specific Diagnostics
  source 1..28: mol_type = other DNA
acagaataaacaccaggtaagaatgagt

SEQ ID NO: 69 - DNA, 22 bp, synthetic construct
  misc_feature 1..22: note = Artificial sequence generated for Engineered CRISPR-Cas System sand Methods for Sensitive and Specific Diagnostics
  source 1..22: mol_type = other DNA
acttttgaagaagctgcgctgt

SEQ ID NO: 70 - DNA, 25 bp, synthetic construct
  misc_feature 1..25: note = Artificial sequence generated for Engineered CRISPR-Cas System sand Methods for Sensitive and Specific Diagnostics
  source 1..25: mol_type = other DNA
tggacagtaaactacgtcatcaagc

SEQ ID NO: 71 - DNA, 22 bp, synthetic construct
  misc_feature 1..22: note = Artificial sequence generated for Engineered CRISPR-Cas System sand Methods for Sensitive and Specific Diagnostics
  source 1..22: mol_type = other DNA
tgttcgcattcaaccaggacag

SEQ ID NO: 72 - DNA, 26 bp, synthetic construct
  misc_feature 1..26: note = Artificial sequence generated for Engineered CRISPR-Cas System sand Methods for Sensitive and Specific Diagnostics
  source 1..26: mol_type = other DNA
acttcatagccacaaggttaaagtca

SEQ ID NO: 73 - DNA, 23 bp, synthetic construct
  misc_feature 1..23: note = Artificial sequence generated for Engineered CRISPR-Cas System sand Methods for Sensitive and Specific Diagnostics
  source 1..23: mol_type = other DNA
acacaccactggttgttactcac

SEQ ID NO: 74 - DNA, 22 bp, synthetic construct
  misc_feature 1..22: note = Artificial sequence generated for Engineered CRISPR-Cas System sand Methods for Sensitive and Specific Diagnostics
  source 1..22: mol_type = other DNA
gtccacactctcctagcaccat

SEQ ID NO: 75 - DNA, 29 bp, synthetic construct
  misc_feature 1..29: note = Artificial sequence generated for Engineered CRISPR-Cas System sand Methods for Sensitive and Specific Diagnostics
  source 1..29: mol_type = other DNA
agtattgccctattttcttcataactggt

SEQ ID NO: 76 - DNA, 22 bp, synthetic construct
  misc_feature 1..22: note = Artificial sequence generated for Engineered CRISPR-Cas System sand Methods for Sensitive and Specific Diagnostics
  source 1..22: mol_type = other DNA
tgtaactggacacattgagccc

SEQ ID NO: 77 - DNA, 23 bp, synthetic construct
  misc_feature 1..23: note = Artificial sequence generated for Engineered CRISPR-Cas System sand Methods for Sensitive and Specific Diagnostics
  source 1..23: mol_type = other DNA
gttcccttccatcatatgcagct

SEQ ID NO: 78 - DNA, 25 bp, synthetic construct
  misc_feature 1..25: note = Artificial sequence generated for Engineered CRISPR-Cas System sand Methods for Sensitive and Specific Diagnostics
  source 1..25: mol_type = other DNA
tggtatgacaaccattagtttggct

SEQ ID NO: 79 - DNA, 22 bp, synthetic construct
  misc_feature 1..22: note = Artificial sequence generated for Engineered CRISPR-Cas System sand Methods for Sensitive and Specific Diagnostics
  source 1..22: mol_type = other DNA
tacgacagatgtcttgtgctgc

SEQ ID NO: 80 - DNA, 22 bp, synthetic construct
  misc_feature 1..22: note = Artificial sequence generated for Engineered CRISPR-Cas System sand Methods for Sensitive and Specific Diagnostics
  source 1..22: mol_type = other DNA
agcagcatctacagcaaaagca

SEQ ID NO: 81 - DNA, 25 bp, synthetic construct
  misc_feature 1..25: note = Artificial sequence generated for Engineered CRISPR-Cas System sand Methods for Sensitive and Specific Diagnostics
  source 1..25: mol_type = other DNA
tacctacaacttgtgctaatgaccc

SEQ ID NO: 82 - DNA, 29 bp, synthetic construct
  misc_feature 1..29: note = Artificial sequence generated for Engineered CRISPR-Cas System sand Methods for Sensitive and Specific Diagnostics
  source 1..29: mol_type = other DNA
agtatgtacaaatacctacaacttgtgct

SEQ ID NO: 83 - DNA, 30 bp, synthetic construct
  misc_feature 1..30: note = Artificial sequence generated for Engineered CRISPR-Cas System sand Methods for Sensitive and Specific Diagnostics
  source 1..30: mol_type = other DNA
aaattgtttcttcatgttggtagttagaga

SEQ ID NO: 84 - DNA, 29 bp, synthetic construct
  misc_feature 1..29: note = Artificial sequence generated for Engineered CRISPR-Cas System sand Methods for Sensitive and Specific Diagnostics
  source 1..29: mol_type = other DNA
ttcatgttggtagttagagaaagtgtgtc

SEQ ID NO: 85 - DNA, 28 bp, synthetic construct
  misc_feature 1..28: note = Artificial sequence generated for Engineered CRISPR-Cas System sand Methods for Sensitive and Specific Diagnostics
  source 1..28: mol_type = other DNA
aggactggtatgattttgtagaaaaccc

SEQ ID NO: 86 - DNA, 28 bp, synthetic construct
  misc_feature 1..28: note = Artificial sequence generated for Engineered CRISPR-Cas System sand Methods for Sensitive and Specific Diagnostics
  source 1..28: mol_type = other DNA
aataacggtcaaagagttttaacctctc

SEQ ID NO: 87 - DNA, 25 bp, synthetic construct
  misc_feature 1..25: note = Artificial sequence generated for Engineered CRISPR-Cas System sand Methods for Sensitive and Specific Diagnostics
  source 1..25: mol_type = other DNA
aggaattacttgtgtatgctgctga

SEQ ID NO: 88 - DNA, 28 bp, synthetic construct
  misc_feature 1..28: note = Artificial sequence generated for Engineered CRISPR-Cas System sand Methods for Sensitive and Specific Diagnostics
  source 1..28: mol_type = other DNA
tgacgatgacttggttagcattaataca

SEQ ID NO: 89 - DNA, 22 bp, synthetic construct
  misc_feature 1..22: note = Artificial sequence generated for Engineered CRISPR-Cas System sand Methods for Sensitive and Specific Diagnostics
  source 1..22: mol_type = other DNA
tcaatagccgccactagaggag

SEQ ID NO: 90 - DNA, 22 bp, synthetic construct
  misc_feature 1..22: note = Artificial sequence generated for Engineered CRISPR-Cas System sand Methods for Sensitive and Specific Diagnostics
  source 1..22: mol_type = other DNA
agtgcattaacattggccgtga

SEQ ID NO: 91 - DNA, 24 bp, synthetic construct
  misc_feature 1..24: note = Artificial sequence generated for Engineered CRISPR-Cas System sand Methods for Sensitive and Specific Diagnostics
  source 1..24: mol_type = other DNA
agcaaaatgttggactgagactga

SEQ ID NO: 92 - DNA, 23 bp, synthetic construct
  misc_feature 1..23: note = Artificial sequence generated for Engineered CRISPR-Cas System sand Methods for Sensitive and Specific Diagnostics
  source 1..23: mol_type = other DNA
agcctcataaaactcaggttccc

SEQ ID NO: 93 - DNA, 28 bp, synthetic construct
  misc_feature 1..28: note = Artificial sequence generated for Engineered CRISPR-Cas System sand Methods for Sensitive and Specific Diagnostics
  source 1..28: mol_type = other DNA
actcaactttacttaggaggtatgagct

SEQ ID NO: 94 - DNA, 29 bp, synthetic construct
  misc_feature 1..29: note = Artificial sequence generated for Engineered CRISPR-Cas System sand Methods for Sensitive and Specific Diagnostics
  source 1..29: mol_type = other DNA
ggtgtactctcctatttgtactttactgt

SEQ ID NO: 95 - DNA, 22 bp, synthetic construct
  misc_feature 1..22: note = Artificial sequence generated for Engineered CRISPR-Cas System sand Methods for Sensitive and Specific Diagnostics
  source 1..22: mol_type = other DNA
attctacactccagggaccacc

SEQ ID NO: 96 - DNA, 22 bp, synthetic construct
  misc_feature 1..22: note = Artificial sequence generated for Engineered CRISPR-Cas System sand Methods for Sensitive and Specific Diagnostics
  source 1..22: mol_type = other DNA
gtaattgagcagggtcgccaat

SEQ ID NO: 97 - DNA, 23 bp, synthetic construct
  misc_feature 1..23: note = Artificial sequence generated for Engineered CRISPR-Cas System sand Methods for Sensitive and Specific Diagnostics
  source 1..23: mol_type = other DNA
tcacgcatgatgtttcatctgca

SEQ ID NO: 98 - DNA, 26 bp, synthetic construct
  misc_feature 1..26: note = Artificial sequence generated for Engineered CRISPR-Cas System sand Methods for Sensitive and Specific Diagnostics
  source 1..26: mol_type = other DNA
aagagtcctgttacattttcagcttg

SEQ ID NO: 99 - DNA, 22 bp, synthetic construct
  misc_feature 1..22: note = Artificial sequence generated for Engineered CRISPR-Cas System sand Methods for Sensitive and Specific Diagnostics
  source 1..22: mol_type = other DNA
tgtttatcacccgcgaagaagc

SEQ ID NO: 100 - DNA, 22 bp, synthetic construct
  misc_feature 1..22: note = Artificial sequence generated for Engineered CRISPR-Cas System sand Methods for Sensitive and Specific Diagnostics
  source 1..22: mol_type = other DNA
atcacatagacaacaggtgcgc

SEQ ID NO: 101 - DNA, 22 bp, synthetic construct
  misc_feature 1..22: note = Artificial sequence generated for Engineered CRISPR-Cas System sand Methods for Sensitive and Specific Diagnostics
  source 1..22: mol_type = other DNA
tgttaagcgtgttgactggact

SEQ ID NO: 102 - DNA, 22 bp, synthetic construct
  misc_feature 1..22: note = Artificial sequence generated for Engineered CRISPR-Cas System sand Methods for Sensitive and Specific Diagnostics
  source 1..22: mol_type = other DNA
acaaactgccaccatcacaacc

SEQ ID NO: 103 - DNA, 22 bp, synthetic construct
  misc_feature 1..22: note = Artificial sequence generated for Engineered CRISPR-Cas System sand Methods for Sensitive and Specific Diagnostics
  source 1..22: mol_type = other DNA
gctggctttagcttgtgggttt

SEQ ID NO: 104 - DNA, 28 bp, synthetic construct
  misc_feature 1..28: note = Artificial sequence generated for Engineered CRISPR-Cas System sand Methods for Sensitive and Specific Diagnostics
  source 1..28: mol_type = other DNA
tgtcagtcatagaacaaacaccaatagt

SEQ ID NO: 105 - DNA, 28 bp, synthetic construct
  misc_feature 1..28: note = Artificial sequence generated for Engineered CRISPR-Cas System sand Methods for Sensitive and Specific Diagnostics
  source 1..28: mol_type = other DNA
gttgtccaacaattacctgaaacttact

SEQ ID NO: 106 - DNA, 30 bp, synthetic construct
  misc_feature 1..30: note = Artificial sequence generated for Engineered CRISPR-Cas System sand Methods for Sensitive and Specific Diagnostics
  source 1..30: mol_type = other DNA
caaccttagaaactacagataaatcttggg

SEQ ID NO: 107 - DNA, 27 bp, synthetic construct
  misc_feature 1..27: note = Artificial sequence generated for Engineered CRISPR-Cas System sand Methods for Sensitive and Specific Diagnostics
  source 1..27: mol_type = other DNA
tgtcgcaaaatatactcaactgtgtca

SEQ ID NO: 108 - DNA, 23 bp, synthetic construct
  misc_feature 1..23: note = Artificial sequence generated for Engineered CRISPR-Cas System sand Methods for Sensitive and Specific Diagnostics
  source 1..23: mol_type = other DNA
tctttatagccacggaacctcca

SEQ ID NO: 109 - DNA, 29 bp, synthetic construct
  misc_feature 1..29: note = Artificial sequence generated for Engineered CRISPR-Cas System sand Methods for Sensitive and Specific Diagnostics
  source 1..29: mol_type = other DNA
acaaatccaattcagttgtcttcctattc

SEQ ID NO: 110 - DNA, 27 bp, synthetic construct
  misc_feature 1..27: note = Artificial sequence generated for Engineered CRISPR-Cas System sand Methods for Sensitive and Specific Diagnostics
  source 1..27: mol_type = other DNA
tggaaaagaaaggtaagaacaagtcct

SEQ ID NO: 111 - DNA, 29 bp, synthetic construct
  misc_feature 1..29: note = Artificial sequence generated for Engineered CRISPR-Cas System sand Methods for Sensitive and Specific Diagnostics
  source 1..29: mol_type = other DNA
caattttgtaatgatccatttttgggtgt

SEQ ID NO: 112 - DNA, 22 bp, synthetic construct
  misc_feature 1..22: note = Artificial sequence generated for Engineered CRISPR-Cas System sand Methods for Sensitive and Specific Diagnostics
  source 1..22: mol_type = other DNA
caccagctgtccaacctgaaga

SEQ ID NO: 113 - DNA, 26 bp, synthetic construct
  misc_feature 1..26: note = Artificial sequence generated for Engineered CRISPR-Cas System sand Methods for Sensitive and Specific Diagnostics
  source 1..26: mol_type = other DNA
agagtccaaccaacagaatctattgt

SEQ ID NO: 114 - DNA, 26 bp, synthetic construct
  misc_feature 1..26: note = Artificial sequence generated for Engineered CRISPR-Cas System sand Methods for Sensitive and Specific Diagnostics
  source 1..26: mol_type = other DNA
accaccaaccttagaatcaagattgt

SEQ ID NO: 115 - DNA, 22 bp, synthetic construct
  misc_feature 1..22: note = Artificial sequence generated for Engineered CRISPR-Cas System sand Methods for Sensitive and Specific Diagnostics
  source 1..22: mol_type = other DNA
ccagcaactgtttgtggaccta

SEQ ID NO: 116 - DNA, 22 bp, synthetic construct
  misc_feature 1..22: note = Artificial sequence generated for Engineered CRISPR-Cas System sand Methods for Sensitive and Specific Diagnostics
  source 1..22: mol_type = other DNA
cagcccctattaaacagcctgc

SEQ ID NO: 117 - DNA, 23 bp, synthetic construct
  misc_feature 1..23: note = Artificial sequence generated for Engineered CRISPR-Cas System sand Methods for Sensitive and Specific Diagnostics
  source 1..23: mol_type = other DNA
gtggtgattcaactgaatgcagc

SEQ ID NO: 118 - DNA, 24 bp, synthetic construct
  misc_feature 1..24: note = Artificial sequence generated for Engineered CRISPR-Cas System sand Methods for Sensitive and Specific Diagnostics
  source 1..24: mol_type = other DNA
catttcatctgtgagcaaaggtgg

SEQ ID NO: 119 - DNA, 25 bp, synthetic construct
  misc_feature 1..25: note = Artificial sequence generated for Engineered CRISPR-Cas System sand Methods for Sensitive and Specific Diagnostics
  source 1..25: mol_type = other DNA
gcacttggaaaacttcaagatgtgg

SEQ ID NO: 120 - DNA, 24 bp, synthetic construct
  misc_feature 1..24: note = Artificial sequence generated for Engineered CRISPR-Cas System sand Methods for Sensitive and Specific Diagnostics
  source 1..24: mol_type = other DNA
gtgaagttcttttcttgtgcaggg

SEQ ID NO: 121 - DNA, 25 bp, synthetic construct
  misc_feature 1..25: note = Artificial sequence generated for Engineered CRISPR-Cas System sand Methods for Sensitive and Specific Diagnostics
  source 1..25: mol_type = other DNA
tcctttgcaacctgaattagactca

SEQ ID NO: 122 - DNA, 22 bp, synthetic construct
  misc_feature 1..22: note = Artificial sequence generated for Engineered CRISPR-Cas System sand Methods for Sensitive and Specific Diagnostics
  source 1..22: mol_type = other DNA
tttgactcctttgagcactggc

SEQ ID NO: 123 - DNA, 22 bp, synthetic construct
  misc_feature 1..22: note = Artificial sequence generated for Engineered CRISPR-Cas System sand Methods for Sensitive and Specific Diagnostics
  source 1..22: mol_type = other DNA
actagcactctccaagggtgtt

SEQ ID NO: 124 - DNA, 25 bp, synthetic construct
  misc_feature 1..25: note = Artificial sequence generated for Engineered CRISPR-Cas System sand Methods for Sensitive and Specific Diagnostics
  source 1..25: mol_type = other DNA
acacagtcttttactccagattccc

SEQ ID NO: 125 - DNA, 22 bp, synthetic construct
  misc_feature 1..22: note = Artificial sequence generated for Engineered CRISPR-Cas System sand Methods for Sensitive and Specific Diagnostics
  source 1..22: mol_type = other DNA
cgactactagcgtgcctttgta

SEQ ID NO: 126 - DNA, 24 bp, synthetic construct
  misc_feature 1..24: note = Artificial sequence generated for Engineered CRISPR-Cas System sand Methods for Sensitive and Specific Diagnostics
  source 1..24: mol_type = other DNA
actaggttccattgttcaaggagc

SEQ ID NO: 127 - DNA, 22 bp, synthetic construct
  misc_feature 1..22: note = Artificial sequence generated for Engineered CRISPR-Cas System sand Methods for Sensitive and Specific Diagnostics
  source 1..22: mol_type = other DNA
gtacgcgttccatgtggtcatt

SEQ ID NO: 128 - DNA, 22 bp, synthetic construct
  misc_feature 1..22: note = Artificial sequence generated for Engineered CRISPR-Cas System sand Methods for Sensitive and Specific Diagnostics
  source 1..22: mol_type = other DNA
cgcgttccatgtggtcattcaa

SEQ ID NO: 129 - DNA, 25 bp, synthetic construct
  misc_feature 1..25: note = Artificial sequence generated for Engineered CRISPR-Cas System sand Methods for Sensitive and Specific Diagnostics
  source 1..25: mol_type = other DNA
acctgaaagtcaacgagatgaaaca

SEQ ID NO: 130 - DNA, 25 bp, synthetic construct
  misc_feature 1..25: note = Artificial sequence generated for Engineered CRISPR-Cas System sand Methods for Sensitive and Specific Diagnostics
  source 1..25: mol_type = other DNA
acgagatgaaacatctgttgtcact

SEQ ID NO: 131 - DNA, 25 bp, synthetic construct
  misc_feature 1..25: note = Artificial sequence generated for Engineered CRISPR-Cas System sand Methods for Sensitive and Specific Diagnostics
  source 1..25: mol_type = other DNA
tcactaccaagagtgtgttagaggt

SEQ ID NO: 132 - DNA, 29 bp, synthetic construct
  misc_feature 1..29: note = Artificial sequence generated for Engineered CRISPR-Cas System sand Methods for Sensitive and Specific Diagnostics
  source 1..29: mol_type = other DNA
ttcaagtgagaaccaaaagataataagca

SEQ ID NO: 133 - DNA, 23 bp, synthetic construct
  misc_feature 1..23: note = Artificial sequence generated for Engineered CRISPR-Cas System sand Methods for Sensitive and Specific Diagnostics
  source 1..23: mol_type = other DNA
tgaggctggttctaaatcaccca

SEQ ID NO: 134 - DNA, 22 bp, synthetic construct
  misc_feature 1..22: note = Artificial sequence generated for Engineered CRISPR-Cas System sand Methods for Sensitive and Specific Diagnostics
  source 1..22: mol_type = other DNA
aggtcttccttgccatgttgag

SEQ ID NO: 135 - DNA, 22 bp, synthetic construct
  misc_feature 1..22: note = Artificial sequence generated for Engineered CRISPR-Cas System sand Methods for Sensitive and Specific Diagnostics
  source 1..22: mol_type = other DNA
tgagggagccttgaatacacca

SEQ ID NO: 136 - DNA, 22 bp, synthetic construct
  misc_feature 1..22: note = Artificial sequence generated for Engineered CRISPR-Cas System sand Methods for Sensitive and Specific Diagnostics
  source 1..22: mol_type = other DNA
cagtacgtttttgccgaggctt

SEQ ID NO: 137 - DNA, 28 bp, synthetic construct
  misc_feature 1..28: note = Artificial sequence generated for Engineered CRISPR-Cas System sand Methods for Sensitive and Specific Diagnostics
  source 1..28: mol_type = other DNA
tggatgacaaagatccaaatttcaaaga

SEQ ID NO: 138 - DNA, 28 bp, synthetic construct
  misc_feature 1..28: note = Artificial sequence generated for Engineered CRISPR-Cas System sand Methods for Sensitive and Specific Diagnostics
  source 1..28: mol_type = other DNA
acacactgattaaagattgctatgtgag

SEQ ID NO: 139 - DNA, 22 bp, synthetic construct
  misc_feature 1..22: note = Artificial sequence generated for Engineered CRISPR-Cas System sand Methods for Sensitive and Specific Diagnostics
  source 1..22: mol_type = other DNA
ctgttttacaggttcgcgacgt

SEQ ID NO: 140 - DNA, 22 bp, synthetic construct
  misc_feature 1..22: note = Artificial sequence generated for Engineered CRISPR-Cas System sand Methods for Sensitive and Specific Diagnostics
  source 1..22: mol_type = other DNA
taaggatcagtgccaagctcgt

SEQ ID NO: 141 - DNA, 22 bp, synthetic construct
  misc_feature 1..22: note = Artificial sequence generated for Engineered CRISPR-Cas System sand Methods for Sensitive and Specific Diagnostics
  source 1..22: mol_type = other DNA
ggtgtatactgctgccgtgaac

SEQ ID NO: 142 - DNA, 25 bp, synthetic construct
  misc_feature 1..25: note = Artificial sequence generated for Engineered CRISPR-Cas System sand Methods for Sensitive and Specific Diagnostics
  source 1..25: mol_type = other DNA
cacaagtagtggcaccttctttagt

SEQ ID NO: 143 - DNA, 22 bp, synthetic construct
  misc_feature 1..22: note = Artificial sequence generated for Engineered CRISPR-Cas System sand Methods for Sensitive and Specific Diagnostics
  source 1..22: mol_type = other DNA
ggtgttgttggagaaggttccg

SEQ ID NO: 144 - DNA, 22 bp, synthetic construct
  misc_feature 1..22: note = Artificial sequence generated for Engineered CRISPR-Cas System sand Methods for Sensitive and Specific Diagnostics
  source 1..22: mol_type = other DNA
tagcggccttctgtaaaacacg

SEQ ID NO: 145 - DNA, 24 bp, synthetic construct
  misc_feature 1..24: note = Artificial sequence generated for Engineered CRISPR-Cas System sand Methods for Sensitive and Specific Diagnostics
  source 1..24: mol_type = other DNA
agagtttcttagagacggttggga

SEQ ID NO: 146 - DNA, 24 bp, synthetic construct
  misc_feature 1..24: note = Artificial sequence generated for Engineered CRISPR-Cas System sand Methods for Sensitive and Specific Diagnostics
  source 1..24: mol_type = other DNA
gcttcaacagcttcactagtaggt

SEQ ID NO: 147 - DNA, 24 bp, synthetic construct
  misc_feature 1..24: note = Artificial sequence generated for Engineered CRISPR-Cas System sand Methods for Sensitive and Specific Diagnostics
  source 1..24: mol_type = other DNA
tgagaagtgctctgcctatacagt

SEQ ID NO: 148 - DNA, 27 bp, synthetic construct
  misc_feature 1..27: note = Artificial sequence generated for Engineered CRISPR-Cas System sand Methods for Sensitive and Specific Diagnostics
  source 1..27: mol_type = other DNA
tcatctaaccaatcttcttcttgctct

SEQ ID NO: 149 - DNA, 22 bp, synthetic construct
  misc_feature 1..22: note = Artificial sequence generated for Engineered CRISPR-Cas System sand Methods for Sensitive and Specific Diagnostics
  source 1..22: mol_type = other DNA
aaacatggaggaggtgttgcag

SEQ ID NO: 150 - DNA, 27 bp, synthetic construct
  misc_feature 1..27: note = Artificial sequence generated for Engineered CRISPR-Cas System sand Methods for Sensitive and Specific Diagnostics
  source 1..27: mol_type = other DNA
ttcactcttcatttccaaaaagcttga

SEQ ID NO: 151 - DNA, 23 bp, synthetic construct
  misc_feature 1..23: note = Artificial sequence generated for Engineered CRISPR-Cas System sand Methods for Sensitive and Specific Diagnostics
  source 1..23: mol_type = other DNA
catccagattctgccactcttgt

SEQ ID NO: 152 - DNA, 24 bp, synthetic construct
  misc_feature 1..24: note = Artificial sequence generated for Engineered CRISPR-Cas System sand Methods for Sensitive and Specific Diagnostics
  source 1..24: mol_type = other DNA
tggcaatcttcatccagattctgc

SEQ ID NO: 153 - DNA, 22 bp, synthetic construct
  misc_feature 1..22: note = Artificial sequence generated for Engineered CRISPR-Cas System sand Methods for Sensitive and Specific Diagnostics
  source 1..22: mol_type = other DNA
agtttccacacagacaggcatt

SEQ ID NO: 154 - DNA, 22 bp, synthetic construct
  misc_feature 1..22: note = Artificial sequence generated for Engineered CRISPR-Cas System sand Methods for Sensitive and Specific Diagnostics
  source 1..22: mol_type = other DNA
tgcgtgtttcttctgcatgtgc

SEQ ID NO: 155 - DNA, 22 bp, synthetic construct
  misc_feature 1..22: note = Artificial sequence generated for Engineered CRISPR-Cas System sand Methods for Sensitive and Specific Diagnostics
  source 1..22: mol_type = other DNA
aatttggaagaagctgctcggt

SEQ ID NO: 156 - DNA, 22 bp, synthetic construct
  misc_feature 1..22: note = Artificial sequence generated for Engineered CRISPR-Cas System sand Methods for Sensitive and Specific Diagnostics
  source 1..22: mol_type = other DNA
cacaacttgcgtgtggaggtta

SEQ ID NO: 157 - DNA, 29 bp, synthetic construct
  misc_feature 1..29: note = Artificial sequence generated for Engineered CRISPR-Cas System sand Methods for Sensitive and Specific Diagnostics
  source 1..29: mol_type = other DNA
tggaaatacccacaagttaatggtttaac

SEQ ID NO: 158 - DNA, 30 bp, synthetic construct
  misc_feature 1..30: note = Artificial sequence generated for Engineered CRISPR-Cas System sand Methods for Sensitive and Specific Diagnostics
  source 1..30: mol_type = other DNA
acttctattaaatgggcagataacaactgt

SEQ ID NO: 159 - DNA, 24 bp, synthetic construct
  misc_feature 1..24: note = Artificial sequence generated for Engineered CRISPR-Cas System sand Methods for Sensitive and Specific Diagnostics
  source 1..24: mol_type = other DNA
agcttgtttaccacacgtacaagg

SEQ ID NO: 160 - DNA, 23 bp, synthetic construct
  misc_feature 1..23: note = Artificial sequence generated for Engineered CRISPR-Cas System sand Methods for Sensitive and Specific Diagnostics
  source 1..23: mol_type = other DNA
gcttgtttaccacacgtacaagg

SEQ ID NO: 161 - DNA, 27 bp, synthetic construct
  misc_feature 1..27: note = Artificial sequence generated for Engineered CRISPR-Cas System sand Methods for Sensitive and Specific Diagnostics
  source 1..27: mol_type = other DNA
acaaagaaaacagttacacaacaacca

SEQ ID NO: 162 - DNA, 25 bp, synthetic construct
  misc_feature 1..25: note = Artificial sequence generated for Engineered CRISPR-Cas System sand Methods for Sensitive and Specific Diagnostics
  source 1..25: mol_type = other DNA
acgtggctttattagttgcattgtt

SEQ ID NO: 163 - DNA, 29 bp, synthetic construct
  misc_feature 1..29: note = Artificial sequence generated for Engineered CRISPR-Cas System sand Methods for Sensitive and Specific Diagnostics
  source 1..29: mol_type = other DNA
actaccgaagttgtaggagacattatact

SEQ ID NO: 164 - DNA, 27 bp, synthetic construct
  misc_feature 1..27: note = Artificial sequence generated for Engineered CRISPR-Cas System sand Methods for Sensitive and Specific Diagnostics
  source 1..27: mol_type = other DNA
acagtattctttgctatagtagtcggc

SEQ ID NO: 165 - DNA, 23 bp, synthetic construct
  misc_feature 1..23: note = Artificial sequence generated for Engineered CRISPR-Cas System sand Methods for Sensitive and Specific Diagnostics
  source 1..23: mol_type = other DNA
aggcatgccttcttactgtactg

SEQ ID NO: 166 - DNA, 26 bp, synthetic construct
  misc_feature 1..26: note = Artificial sequence generated for Engineered CRISPR-Cas System sand Methods for Sensitive and Specific Diagnostics
  source 1..26: mol_type = other DNA
acattctaaccatagctgaaatcggg

SEQ ID NO: 167 - DNA, 25 bp, synthetic construct
  misc_feature 1..25: note = Artificial sequence generated for Engineered CRISPR-Cas System sand Methods for Sensitive and Specific Diagnostics
  source 1..25: mol_type = other DNA
ttgtgatacattctgtgctggtagt

SEQ ID NO: 168 - DNA, 22 bp, synthetic construct
  misc_feature 1..22: note = Artificial sequence generated for Engineered CRISPR-Cas System sand Methods for Sensitive and Specific Diagnostics
  source 1..22: mol_type = other DNA
tccgcactatcaccaacatcag

SEQ ID NO: 169 - DNA, 26 bp, synthetic construct
  misc_feature 1..26: note = Artificial sequence generated for Engineered CRISPR-Cas System sand Methods for Sensitive and Specific Diagnostics
  source 1..26: mol_type = other DNA
acatagaagttactggcgatagttgt

SEQ ID NO: 170 - DNA, 26 bp, synthetic construct
  misc_feature 1..26: note = Artificial sequence generated for Engineered CRISPR-Cas System sand Methods for Sensitive and Specific Diagnostics
  source 1..26: mol_type = other DNA
tgtttagacatgacatgaacaggtgt

SEQ ID NO: 171 - DNA, 25 bp, synthetic construct
  misc_feature 1..25: note = Artificial sequence generated for Engineered CRISPR-Cas System sand Methods for Sensitive and Specific Diagnostics
  source 1..25: mol_type = other DNA
gcacaactaatggtgactttttgca

SEQ ID NO: 172 - DNA, 26 bp, synthetic construct
  misc_feature 1..26: note = Artificial sequence generated for Engineered CRISPR-Cas System sand Methods for Sensitive and Specific Diagnostics
  source 1..26: mol_type = other DNA
accactagtagatacacaaacaccag

SEQ ID NO: 173 - DNA, 25 bp, synthetic construct
  misc_feature 1..25: note = Artificial sequence generated for Engineered CRISPR-Cas System sand Methods for Sensitive and Specific Diagnostics
  source 1..25: mol_type = other DNA
tggtgaatacagtcatgtagttgcc

SEQ ID NO: 174 - DNA, 24 bp, synthetic construct
  misc_feature 1..24: note = Artificial sequence generated for Engineered CRISPR-Cas System sand Methods for Sensitive and Specific Diagnostics
  source 1..24: mol_type = other DNA
agcacatcactacgcaactttaga

SEQ ID NO: 175 - DNA, 23 bp, synthetic construct
  misc_feature 1..23: note = Artificial sequence generated for Engineered CRISPR-Cas System sand Methods for Sensitive and Specific Diagnostics
  source 1..23: mol_type = other DNA
tcccatctggtaaagttgagggt

SEQ ID NO: 176 - DNA, 22 bp, synthetic construct
  misc_feature 1..22: note = Artificial sequence generated for Engineered CRISPR-Cas System sand Methods for Sensitive and Specific Diagnostics
  source 1..22: mol_type = other DNA
agtgaaattgggcctcatagca

SEQ ID NO: 177 - DNA, 22 bp, synthetic construct
  misc_feature 1..22: note = Artificial sequence generated for Engineered CRISPR-Cas System sand Methods for Sensitive and Specific Diagnostics
  source 1..22: mol_type = other DNA
ttagcttggttgtacgctgctg

SEQ ID NO: 178 - DNA, 26 bp, synthetic construct
  misc_feature 1..26: note = Artificial sequence generated for Engineered CRISPR-Cas System sand Methods for Sensitive and Specific Diagnostics
  source 1..26: mol_type = other DNA
gaacaaagaccattgagtactctgga

SEQ ID NO: 179 - DNA, 25 bp, synthetic construct
  misc_feature 1..25: note = Artificial sequence generated for Engineered CRISPR-Cas System sand Methods for Sensitive and Specific Diagnostics
  source 1..25: mol_type = other DNA
actgtgttatgtatgcatcagctgt

SEQ ID NO: 180 - DNA, 25 bp, synthetic construct
  misc_feature 1..25: note = Artificial sequence generated for Engineered CRISPR-Cas System sand Methods for Sensitive and Specific Diagnostics
  source 1..25: mol_type = other DNA
caccaagagtcagtctaaagtagcg

SEQ ID NO: 181 - DNA, 26 bp, synthetic construct
  misc_feature 1..26: note = Artificial sequence generated for Engineered CRISPR-Cas System sand Methods for Sensitive and Specific Diagnostics
  source 1..26: mol_type = other DNA
tgcacatcagtagtcttactctcagt

SEQ ID NO: 182 - DNA, 22 bp, synthetic construct
  misc_feature 1..22: note = Artificial sequence generated for Engineered CRISPR-Cas System sand Methods for Sensitive and Specific Diagnostics
  source 1..22: mol_type = other DNA
catggctgcatcacggtcaaat

SEQ ID NO: 183 - DNA, 22 bp, synthetic construct
  misc_feature 1..22: note = Artificial sequence generated for Engineered CRISPR-Cas System sand Methods for Sensitive and Specific Diagnostics
  source 1..22: mol_type = other DNA
tgcaagagatggttgtgttccc

SEQ ID NO: 184 - DNA, 23 bp, synthetic construct
  misc_feature 1..23: note = Artificial sequence generated for Engineered CRISPR-Cas System sand Methods for Sensitive and Specific Diagnostics
  source 1..23: mol_type = other DNA
cctacctccctttgttgtgttgt

SEQ ID NO: 185 - DNA, 22 bp, synthetic construct
  misc_feature 1..22: note = Artificial sequence generated for Engineered CRISPR-Cas System sand Methods for Sensitive and Specific Diagnostics
  source 1..22: mol_type = other DNA
tgccacagtacgtctacaagct

SEQ ID NO: 186 - DNA, 22 bp, synthetic construct
  misc_feature 1..22: note = Artificial sequence generated for Engineered CRISPR-Cas System sand Methods for Sensitive and Specific Diagnostics
  source 1..22: mol_type = other DNA
ccacagtacgtctacaagctgg

SEQ ID NO: 187 - DNA, 22 bp, synthetic construct
  misc_feature 1..22: note = Artificial sequence generated for Engineered CRISPR-Cas System sand Methods for Sensitive and Specific Diagnostics
  source 1..22: mol_type = other DNA
aacctttccacataccgcagac

SEQ ID NO: 188 - DNA, 22 bp, synthetic construct
  misc_feature 1..22: note = Artificial sequence generated for Engineered CRISPR-Cas System sand Methods for Sensitive and Specific Diagnostics
  source 1..22: mol_type = other DNA
cgcagacggtacagactgtgtt

SEQ ID NO: 189 - DNA, 22 bp, synthetic construct
  misc_feature 1..22: note = Artificial sequence generated for Engineered CRISPR-Cas System sand Methods for Sensitive and Specific Diagnostics
  source 1..22: mol_type = other DNA
tgtcgcttccaagaaaaggacg

SEQ ID NO: 190 - DNA, 23 bp, synthetic construct
  misc_feature 1..23: note = Artificial sequence generated for Engineered CRISPR-Cas System sand Methods for Sensitive and Specific Diagnostics
  source 1..23: mol_type = other DNA
cgcttccaagaaaaggacgaaga

SEQ ID NO: 191 - DNA, 22 bp, synthetic construct
  misc_feature 1..22: note = Artificial sequence generated for Engineered CRISPR-Cas System sand Methods for Sensitive and Specific Diagnostics
  source 1..22: mol_type = other DNA
cacgttcacctaagttggcgta

SEQ ID NO: 192 - DNA, 23 bp, synthetic construct
  misc_feature 1..23: note = Artificial sequence generated for Engineered CRISPR-Cas System sand Methods for Sensitive and Specific Diagnostics
  source 1..23: mol_type = other DNA
cacgttcacctaagttggcgtat

SEQ ID NO: 193 - DNA, 25 bp, synthetic construct
  misc_feature 1..25: note = Artificial sequence generated for Engineered CRISPR-Cas System sand Methods for Sensitive and Specific Diagnostics
  source 1..25: mol_type = other DNA
tgttgacactgacttaacaaagcct

SEQ ID NO: 194 - DNA, 22 bp, synthetic construct
  misc_feature 1..22: note = Artificial sequence generated for Engineered CRISPR-Cas System sand Methods for Sensitive and Specific Diagnostics
  source 1..22: mol_type = other DNA
tagattaccagaagcagcgtgc

SEQ ID NO: 195 - DNA, 30 bp, synthetic construct
  misc_feature 1..30: note = Artificial sequence generated for Engineered CRISPR-Cas System sand Methods for Sensitive and Specific Diagnostics
  source 1..30: mol_type = other DNA
gttgataagtactttgattgttacgatggt

SEQ ID NO: 196 - DNA, 22 bp, synthetic construct
  misc_feature 1..22: note = Artificial sequence generated for Engineered CRISPR-Cas System sand Methods for Sensitive and Specific Diagnostics
  source 1..22: mol_type = other DNA
taacatgttgtgccaaccacca

SEQ ID NO: 197 - DNA, 22 bp, synthetic construct
  misc_feature 1..22: note = Artificial sequence generated for Engineered CRISPR-Cas System sand Methods for Sensitive and Specific Diagnostics
  source 1..22: mol_type = other DNA
catcaggagatgccacaactgc

SEQ ID NO: 198 - DNA, 25 bp, synthetic construct
  misc_feature 1..25: note = Artificial sequence generated for Engineered CRISPR-Cas System sand Methods for Sensitive and Specific Diagnostics
  source 1..25: mol_type = other DNA
gttgagagcaaaattcatgaggtcc

SEQ ID NO: 199 - DNA, 26 bp, synthetic construct
  misc_feature 1..26: note = Artificial sequence generated for Engineered CRISPR-Cas System sand Methods for Sensitive and Specific Diagnostics
  source 1..26: mol_type = other DNA
tgagttaacaggacacatgttagaca

SEQ ID NO: 200 - DNA, 25 bp, synthetic construct
  misc_feature 1..25: note = Artificial sequence generated for Engineered CRISPR-Cas System sand Methods for Sensitive and Specific Diagnostics
  source 1..25: mol_type = other DNA
aaccaaaaacttgtccattagcaca

SEQ ID NO: 201 - DNA, 22 bp, synthetic construct
  misc_feature 1..22: note = Artificial sequence generated for Engineered CRISPR-Cas System sand Methods for Sensitive and Specific Diagnostics
  source 1..22: mol_type = other DNA
acctagaccaccacttaaccga

SEQ ID NO: 202 - DNA, 22 bp, synthetic construct
  misc_feature 1..22: note = Artificial sequence generated for Engineered CRISPR-Cas System sand Methods for Sensitive and Specific Diagnostics
  source 1..22: mol_type = other DNA
acactatgcgagcagaagggta

SEQ ID NO: 203 - DNA, 25 bp, synthetic construct
  misc_feature 1..25: note = Artificial sequence generated for Engineered CRISPR-Cas System sand Methods for Sensitive and Specific Diagnostics
  source 1..25: mol_type = other DNA
tgatttgagtgttgtcaatgccaga

SEQ ID NO: 204 - DNA, 23 bp, synthetic construct
  misc_feature 1..23: note = Artificial sequence generated for Engineered CRISPR-Cas System sand Methods for Sensitive and Specific Diagnostics
  source 1..23: mol_type = other DNA
cttttctccaagcagggttacgt

SEQ ID NO: 205 - DNA, 27 bp, synthetic construct
  misc_feature 1..27: note = Artificial sequence generated for Engineered CRISPR-Cas System sand Methods for Sensitive and Specific Diagnostics
  source 1..27: mol_type = other DNA
tgatagagacctttatgacaagttgca

SEQ ID NO: 206 - DNA, 24 bp, synthetic construct
  misc_feature 1..24: note = Artificial sequence generated for Engineered CRISPR-Cas System sand Methods for Sensitive and Specific Diagnostics
  source 1..24: mol_type = other DNA
ggtaccaacagcttctctagtagc

SEQ ID NO: 207 - DNA, 22 bp, synthetic construct
  misc_feature 1..22: note = Artificial sequence generated for Engineered CRISPR-Cas System sand Methods for Sensitive and Specific Diagnostics
  source 1..22: mol_type = other DNA
ggcacatggctttgagttgaca

SEQ ID NO: 208 - DNA, 22 bp, synthetic construct
  misc_feature 1..22: note = Artificial sequence generated for Engineered CRISPR-Cas System sand Methods for Sensitive and Specific Diagnostics
  source 1..22: mol_type = other DNA
gttgaacctttctacaagccgc

SEQ ID NO: 209 - DNA, 28 bp, synthetic construct
  misc_feature 1..28: note = Artificial sequence generated for Engineered CRISPR-Cas System sand Methods for Sensitive and Specific Diagnostics
  source 1..28: mol_type = other DNA
tcgatagatatcctgctaattccattgt

SEQ ID NO: 210 - DNA, 25 bp, synthetic construct
  misc_feature 1..25: note = Artificial sequence generated for Engineered CRISPR-Cas System sand Methods for Sensitive and Specific Diagnostics
  source 1..25: mol_type = other DNA
agtcttgtaaaagtgttccagaggt

SEQ ID NO: 211 - DNA, 22 bp, synthetic construct
  misc_feature 1..22: note = Artificial sequence generated for Engineered CRISPR-Cas System sand Methods for Sensitive and Specific Diagnostics
  source 1..22: mol_type = other DNA
gggtgtggacattgctgctaat

SEQ ID NO: 212 - DNA, 24 bp, synthetic construct
  misc_feature 1..24: note = Artificial sequence generated for Engineered CRISPR-Cas System sand Methods for Sensitive and Specific Diagnostics
  source 1..24: mol_type = other DNA
tcaatttccatttgactcctgggt

SEQ ID NO: 213 - DNA, 24 bp, synthetic construct
  misc_feature 1..24: note = Artificial sequence generated for Engineered CRISPR-Cas System sand Methods for Sensitive and Specific Diagnostics
  source 1..24: mol_type = other DNA
acaggttcatctaagtgtgtgtgt

SEQ ID NO: 214 - DNA, 23 bp, synthetic construct
  misc_feature 1..23: note = Artificial sequence generated for Engineered CRISPR-Cas System sand Methods for Sensitive and Specific Diagnostics
  source 1..23: mol_type = other DNA
ctcctttatcagaaccagcacca

SEQ ID NO: 215 - DNA, 29 bp, synthetic construct
  misc_feature 1..29: note = Artificial sequence generated for Engineered CRISPR-Cas System sand Methods for Sensitive and Specific Diagnostics
  source 1..29: mol_type = other DNA
acaaaagaaaatgactctaaagagggttt

SEQ ID NO: 216 - DNA, 28 bp, synthetic construct
  misc_feature 1..28: note = Artificial sequence generated for Engineered CRISPR-Cas System sand Methods for Sensitive and Specific Diagnostics
  source 1..28: mol_type = other DNA
tgaccttcttttaaagacataacagcag

SEQ ID NO: 217 - DNA, 24 bp, synthetic construct
  misc_feature 1..24: note = Artificial sequence generated for Engineered CRISPR-Cas System sand Methods for Sensitive and Specific Diagnostics
  source 1..24: mol_type = other DNA
acacgtggtgtttattaccctgac

SEQ ID NO: 218 - DNA, 25 bp, synthetic construct
  misc_feature 1..25: note = Artificial sequence generated for Engineered CRISPR-Cas System sand Methods for Sensitive and Specific Diagnostics
  source 1..25: mol_type = other DNA
actctgaactcactttccatccaac

SEQ ID NO: 219 - DNA, 28 bp, synthetic construct
  misc_feature 1..28: note = Artificial sequence generated for Engineered CRISPR-Cas System sand Methods for Sensitive and Specific Diagnostics
  source 1..28: mol_type = other DNA
acatcactaggtttcaaactttacttgc

SEQ ID NO: 220 - DNA, 24 bp, synthetic construct
  misc_feature 1..24: note = Artificial sequence generated for Engineered CRISPR-Cas System sand Methods for Sensitive and Specific Diagnostics
  source 1..24: mol_type = other DNA
gcaacacagttgctgattctcttc

SEQ ID NO: 221 - DNA, 22 bp, synthetic construct
  misc_feature 1..22: note = Artificial sequence generated for Engineered CRISPR-Cas System sand Methods for Sensitive and Specific Diagnostics
  source 1..22: mol_type = other DNA
agggcaaactggaaagattgct

SEQ ID NO: 222 - DNA, 23 bp, synthetic construct
  misc_feature 1..23: note = Artificial sequence generated for Engineered CRISPR-Cas System sand Methods for Sensitive and Specific Diagnostics
  source 1..23: mol_type = other DNA
gggcaaactggaaagattgctga

SEQ ID NO: 223 - DNA, 22 bp, synthetic construct
  misc_feature 1..22: note = Artificial sequence generated for Engineered CRISPR-Cas System sand Methods for Sensitive and Specific Diagnostics
  source 1..22: mol_type = other DNA
acacctgtgcctgttaaaccat

SEQ ID NO: 224 - DNA, 23 bp, synthetic construct
  misc_feature 1..23: note = Artificial sequence generated for Engineered CRISPR-Cas System sand Methods for Sensitive and Specific Diagnostics
  source 1..23: mol_type = other DNA
acctgtgcctgttaaaccattga

SEQ ID NO: 225 - DNA, 23 bp, synthetic construct
  misc_feature 1..23: note = Artificial sequence generated for Engineered CRISPR-Cas System sand Methods for Sensitive and Specific Diagnostics
  source 1..23: mol_type = other DNA
caacttactcctacttggcgtgt

SEQ ID NO: 226 - DNA, 25 bp, synthetic construct
  misc_feature 1..25: note = Artificial sequence generated for Engineered CRISPR-Cas System sand Methods for Sensitive and Specific Diagnostics
  source 1..25: mol_type = other DNA
tgtgtacaaaaactgccatattgca

SEQ ID NO: 227 - DNA, 22 bp, synthetic construct
  misc_feature 1..22: note = Artificial sequence generated for Engineered CRISPR-Cas System sand Methods for Sensitive and Specific Diagnostics
  source 1..22: mol_type = other DNA
ttgccttggtgatattgctgct

SEQ ID NO: 228 - DNA, 24 bp, synthetic construct
  misc_feature 1..24: note = Artificial sequence generated for Engineered CRISPR-Cas System sand Methods for Sensitive and Specific Diagnostics
  source 1..24: mol_type = other DNA
tggagctaagttgtttaacaagcg

SEQ ID NO: 229 - DNA, 25 bp, synthetic construct
  misc_feature 1..25: note = Artificial sequence generated for Engineered CRISPR-Cas System sand Methods for Sensitive and Specific Diagnostics
  source 1..25: mol_type = other DNA
gggctatcatcttatgtccttccct

SEQ ID NO: 230 - DNA, 24 bp, synthetic construct
  misc_feature 1..24: note = Artificial sequence generated for Engineered CRISPR-Cas System sand Methods for Sensitive and Specific Diagnostics
  source 1..24: mol_type = other DNA
tgccagagatgtcacctaaatcaa

SEQ ID NO: 231 - DNA, 22 bp, synthetic construct
  misc_feature 1..22: note = Artificial sequence generated for Engineered CRISPR-Cas System sand Methods for Sensitive and Specific Diagnostics
  source 1..22: mol_type = other DNA
tgctgtagttgtctcaagggct

SEQ ID NO: 232 - DNA, 27 bp, synthetic construct
  misc_feature 1..27: note = Artificial sequence generated for Engineered CRISPR-Cas System sand Methods for Sensitive and Specific Diagnostics
  source 1..27: mol_type = other DNA
aggtgtgagtaaactgttacaaacaac

SEQ ID NO: 233 - DNA, 22 bp, synthetic construct
  misc_feature 1..22: note = Artificial sequence generated for Engineered CRISPR-Cas System sand Methods for Sensitive and Specific Diagnostics
  source 1..22: mol_type = other DNA
tcaggtgatggcacaacaagtc

SEQ ID NO: 234 - DNA, 25 bp, synthetic construct
  misc_feature 1..25: note = Artificial sequence generated for Engineered CRISPR-Cas System sand Methods for Sensitive and Specific Diagnostics
  source 1..25: mol_type = other DNA
acgaaagcaagaaaaagaagtacgc

SEQ ID NO: 235 - DNA, 22 bp, synthetic construct
  misc_feature 1..22: note = Artificial sequence generated for Engineered CRISPR-Cas System sand Methods for Sensitive and Specific Diagnostics
  source 1..22: mol_type = other DNA
ccatggcagattccaacggtac

SEQ ID NO: 236 - DNA, 23 bp, synthetic construct
  misc_feature 1..23: note = Artificial sequence generated for Engineered CRISPR-Cas System sand Methods for Sensitive and Specific Diagnostics
  source 1..23: mol_type = other DNA
tggtcagaatagtgccatggagt

SEQ ID NO: 237 - DNA, 23 bp, synthetic construct
  misc_feature 1..23: note = Artificial sequence generated for Engineered CRISPR-Cas System sand Methods for Sensitive and Specific Diagnostics
  source 1..23: mol_type = other DNA
acacagaccattccagtagcagt

SEQ ID NO: 238 - DNA, 22 bp, synthetic construct
  misc_feature 1..22: note = Artificial sequence generated for Engineered CRISPR-Cas System sand Methods for Sensitive and Specific Diagnostics
  source 1..22: mol_type = other DNA
tgaaatggtgaattgccctcgt

SEQ ID NO: 239 - DNA, 24 bp, synthetic construct
  misc_feature 1..24: note = Artificial sequence generated for Engineered CRISPR-Cas System sand Methods for Sensitive and Specific Diagnostics
  source 1..24: mol_type = other DNA
tttgtgctttttagcctttctgct

SEQ ID NO: 240 - DNA, 27 bp, synthetic construct
  misc_feature 1..27: note = Artificial sequence generated for Engineered CRISPR-Cas System sand Methods for Sensitive and Specific Diagnostics
  source 1..27: mol_type = other DNA
aggttcctggcaattaattgtaaaagg

SEQ ID NO: 241 - DNA, 22 bp, synthetic construct
  misc_feature 1..22: note = Artificial sequence generated for Engineered CRISPR-Cas System sand Methods for Sensitive and Specific Diagnostics
  source 1..22: mol_type = other DNA
ggccccaaggtttacccaataa

SEQ ID NO: 242 - DNA, 23 bp, synthetic construct
  misc_feature 1..23: note = Artificial sequence generated for Engineered CRISPR-Cas System sand Methods for Sensitive and Specific Diagnostics
  source 1..23: mol_type = other DNA
tttggcaatgttgttccttgagg

SEQ ID NO: 243 - DNA, 22 bp, synthetic construct
  misc_feature 1..22: note = Artificial sequence generated for Engineered CRISPR-Cas System sand Methods for Sensitive and Specific Diagnostics
  source 1..22: mol_type = other DNA
gccaacaacaacaaggccaaac

SEQ ID NO: 244 - DNA, 22 bp, synthetic construct
  misc_feature 1..22: note = Artificial sequence generated for Engineered CRISPR-Cas System sand Methods for Sensitive and Specific Diagnostics
  source 1..22: mol_type = other DNA
taggctctgttggtgggaatgt

SEQ ID NO: 245 - DNA, 24 bp, synthetic construct
  misc_feature 1..24: note = Artificial sequence generated for Engineered CRISPR-Cas System sand Methods for Sensitive and Specific Diagnostics
  source 1..24: mol_type = other DNA
aacaattgcaacaatccatgagca

SEQ ID NO: 246 - DNA, 30 bp, synthetic construct
  misc_feature 1..30: note = Artificial sequence generated for Engineered CRISPR-Cas System sand Methods for Sensitive and Specific Diagnostics
  source 1..30: mol_type = other DNA
ttctcctaagaagctattaaaatcacatgg

(19) United States
(12) Patent Application Publication
(10) Pub. No.: US 2023/0057482 A1
(43) Pub. Date: Feb. 23, 2023

(54) ENGINEERED CRISPR-CAS SYSTEMS AND METHODS FOR SENSITIVE AND SPECIFIC DIAGNOSTICS

(71) Applicant: Montana State University, Bozeman, MT (US)

(72) Inventors: Blake A. WIEDENHEFT, Bozeman, MT (US); Andrew SANTIAGO-FRANGOS, Bozeman, MT (US); Anna A. NEMUDRAIA, Bozeman, MT (US); Artem A. NEMUDRYI, Bozeman, MT (US)

(21) Appl. No.: 17/814,674

(22) Filed: Jul. 25, 2022

Related U.S. Application Data
(63) Continuation of application No. 17/240,858, filed on Apr. 26, 2021.
(60) Provisional application No. 63/320,199, filed on Mar. 15, 2022.
(60) Provisional application No. 63/016,081, filed on Apr. 27, 2020.

(51) Int. Cl. C12Q 1/70; C12N 9/22; C12N 15/11

(52) U.S. Cl. CPC C12Q 1/701; C12N 9/22; C12N 15/11; C12N 2800/80; C12N 2310/20

(57) ABSTRACT

The disclosure relates to an engineered type III CRISPR-Cas system for sensitive and sequence specific detection of nucleic acid in a sample. For example, the engineered type III CRISPR-Cas system may be implemented as an assay for testing SARS-CoV-2 virus (or other target nucleic acid in the sample) that can be performed quickly, such as in one hour or less. Nucleic acid recognition by type III systems may trigger Cas10-mediated nuclease activity and/or polymerase activity, which may generate pyrophosphates, protons and cyclic oligonucleotides. The nuclease activity and/or the one or more products of the Cas10-polymerase are detected using colorimetric, visible fluorometric, and/or instrumented fluorometric detection.

### CROSS-REFERENCE TO RELATED APPLICATIONS

This patent application is a continuation of U.S. patent application Ser. No. 17/240,858, filed on Apr. 26, 2021, and claims the benefit of priority of U.S. Provisional Application No. 63/016,081, filed on Apr. 27, 2020, U.S. Provisional Application No. 63/046,936, filed on Jul. 1, 2020, U.S. Provisional Application No. 63/047,598, filed on Jul. 2, 2020, U.S. Provisional Application No. 63/065,094, filed on Aug. 13, 2020, U.S. Provisional Application No. 63/065,626, filed on Aug. 14, 2020, U.S. Provisional Application No. 63/080,128, filed on Sep. 18, 2020, and U.S. Provisional Application No. 63/157,568, filed on Mar. 5, 2021, each of which is incorporated by reference herein for all purposes.

### SEQUENCES

A sequence listing, named “065869-0569568_SEQUENCE_LISTING.xml”, created Oct. 17, 2022, 337,122 bytes, in XML format accompanies this application. The sequence listing is incorporated by reference in its entirety herein for all purposes.

### BACKGROUND

Frequent tests and quick results are critical for stopping the spread of SARS-CoV-2 and ending the current COVID-19 pandemic C. RT-qPCR (reverse transcriptase-quantitative polymerase chain reaction) has been the gold standard for viral diagnostics, but this method is slow and requires sophisticated equipment that is expensive to purchase and operate. Thus, there is an urgent need for inexpensive new technologies that enable fast, reliable, and scalable detection of viruses.

### SUMMARY

The disclosure relates to methods and engineered systems to detect presence of nucleic acid in a sample. In some examples, the nucleic acid may be associated with disease of a subject that provided the sample, which may include bodily fluid and/or other type of sample. In some examples, the nucleic acid may be ribonucleic acid (RNA) or deoxyribonucleic acid (DNA). In some examples, the nucleic acid is from one or more genomes of a pathogen. In some examples, the pathogen may be a virus. In some examples, the pathogen may an RNA from bacteria. Various examples disclosed herein may be described in the context of detecting severe acute respiratory syndrome coronavirus 2 (SARS-CoV-2) that causes the Coronavirus Disease 2019 (COVID-19) in humans, although other nucleic acids may be detected. Furthermore, subjects other than humans may be tested based on the disclosures herein. It should be further understood that particular nucleic acids may be detected based on the disclosures herein, whether such nucleic acid to be detected originates from a pathogen or subject being tested. For example, the genetic makeup such as mutations or subject-specific sequences may be detected based on the disclosures herein.

One general aspect includes an engineered system to detect nucleic acid (NA) in a sample. The engineered system also includes an engineered type III clustered regularly interspaced short palindromic repeat (CRISPR)-Cas system to detect NA in the sample, the engineered CRISPR-Cas system may include: may include a CRISPR RNA-guide sequence that is complementary to a locus of the nucleic acid; a first subunit that undergoes a conformational change upon binding of the engineered type III CRISPR-Cas system to the locus of the nucleic acid, the conformational change activating DNase activity of the first subunit and/or polymerase activity of the first subunit, the polymerase activity generating one or more products. The system also includes a detection system to detect the DNase activity and/or the one or more products of the polymerase activity.

Implementations may include one or more of the following features. The nucleic acid may include a viral RNA. The viral RNA may include RNA of severe acute respiratory syndrome coronavirus 2 (SARS-CoV-2). The locus may include a nucleocapsid gene (n-gene) of the SARS-CoV-2. The locus may include a region of the viral RNA that is conserved among a plurality of SARS-CoV-2 genomes. The CRISPR guide sequence may include a nucleic acid sequence of SEQ ID NO. 1.

The one or more products may include a linear or cyclic oligonucleotide and where the detection system may include instrumented fluorometric detection may include: an RNA tether linking a fluorophore to a quencher; and a nuclease activated by the linear or cyclic oligonucleotide, the activated nuclease cleaving the RNA tether to thereby release the fluorophore that is detected by a fluorescence detecting instrument. The linear or cyclic oligonucleotide may include a cyclic oligoadenylate, and where the nuclease activated by the linear or cyclic oligonucleotide may include Csm6. The instrumented fluorometric detection further may include: a DNA tether linking the fluorophore or a second fluorophore to the quencher or a second quencher, where the DNase activity cleaves the DNA tether to thereby release the fluorophore or the second fluorophore. The detection system may include instrumented fluorometric detection may include: a DNA tether linking a fluorophore to a quencher, where the first subunit has a DNase activity that is activated upon hybridization of the RNA guide to the locus of the viral RNA, the DNase activity cleaving the DNA tether to thereby release the fluorophore that is detected. The one or more products may include a linear or cyclic oligonucleotide and where the detection system may include instrumented fluorometric detection may include: a DNA tether linking a fluorophore to a quencher; and a nuclease activated by the linear or cyclic oligonucleotide, the activated nuclease cleaving the DNA tether to thereby release the fluorophore that is detected by a fluorescence detecting instrument. The one or more products may include a pyrophosphate, and where the detection system may include visible fluorometric detection may include: a fluorescent dye quenched by a quencher; where the pyrophosphate forms an insoluble precipitate with the quencher to thereby unquench the fluorescent dye that is detected based on a color change. The fluorescent molecule may be calcein and the quencher may include manganese, and where unquenched calcein is bound by magnesium to form a fluorescent complex that is detected.

The first subunit may include a Cas10 subunit, the second subunit may include Csm3, and where an activity of the Cas10 subunit is moderated by activity of the second subunit in the wildtype form, and where the introduced mutation to the second subunit disrupts the moderation of the Cas10 subunit. The one or more products may include protons, and where the detection system may include a colorimetric system, the colorimetric system may include: a solution may include a pH-sensitive dye; and where the protons acidify the solution, resulting in a change in color of the pH-sensitive dye. The engineered type III CRISPR-Cas system further may include: an engineered second subunit may include a backbone subunit of the engineered type III CRISPR-Cas system with an introduced mutation, the engineered second subunit having RNase activity when in wildtype form, but the introduced mutation disrupting the RNase activity to prevent degradation of the viral RNA, thereby increasing signal generation of the detection system. The wildtype form of the second subunit may include an amino acid sequence of SEQ ID NO. 26 and the second subunit with the introduced mutation may include an amino acid sequence of SEQ ID NO. 27.

The one or more products may include (i) a linear or cyclic oligonucleotide, (ii) protons, and (iii) pyrophosphates where the detection system may include: fluorometric detection may include: an RNA tether linking a fluorophore to a quencher; a nuclease activated by the linear or cyclic oligonucleotide, the activated nuclease cleaving the RNA tether to thereby release the fluorophore that is detected; and colorimetric detection may include: a solution may include a pH-sensitive dye; and where the solution is acidified by the protons resulting in a change in color of the pH-sensitive dye. The fluorometric detection further may include: a DNA tether linking the fluorophore or a second fluorophore to the quencher or a second quencher, where the DNase activity cleaves the DNA tether to thereby release the fluorophore or the second fluorophore. The one or more products may include protons, where the detection system may include: fluorometric detection may include: a DNA tether linking a fluorophore to a quencher, where the DNase activity cleaves the DNA tether to thereby release the fluorophore that is detected; and colorimetric detection may include: a solution may include a pH-sensitive dye; and where the solution is acidified by the protons resulting in a change in color of the pH-sensitive dye. The nucleic acid may include RNA, the system may include: a reverse transcription loop-mediated isothermal amplification (RT-LAMP) primer having a T7 binding site for RT-LAMP-T7 amplification of the RNA. The RT-LAMP-T7 amplification and the detection of the RNA may include a single pot combination.

One general aspect includes a method of detecting nucleic acid in a sample based on an engineered type III clustered regularly interspaced short palindromic repeat (CRISPR)-Cas system. The method of detecting nucleic acid also includes contacting the sample with the engineered type III CRISPR-Cas system, the engineered type III CRISPR-Cas system may include: a first subunit, and a CRISPR guide may include a CRISPR guide sequence engineered to be complementary to a locus of the nucleic acid. When the engineered CRISPR-Cas system binds to the nucleic acid at the locus via the CRISPR guide, the first subunit undergoes a conformational change that activates a nuclease activity and/or a polymerase activity of the first subunit; and detecting the nuclease activity and/or one or more products of the polymerase activity.

Implementations may include one or more of the following features. The method where the nucleic acid may include a viral RNA. The viral RNA may include RNA of severe acute respiratory syndrome coronavirus 2 (SARS-CoV-2). The method may include: prior to contacting the sample with the engineered type III CRISPR-Cas system, amplifying the viral RNA with an isothermal amplification. The isothermal amplification may include a reverse transcription loop-mediated isothermal amplification based on primers directed to the locus, the primers including a T7 promotor site for T7 RNA polymerization. The viral RNA is amplified without a polymerase chain reaction (PCR). The type III engineered CRISPR-Cas system may include a Csm3 subunit that cleaves the viral RNA, the method may include: introducing a mutation to the Csm3 subunit in the engineered CRISPR-Cas system to prevent degradation of the viral RNA. Contacting the sample with the engineered type III CRISPR-Cas system may include: contacting the sample with a fluorophore and a quencher tethered together by a nucleic acid tether, where the conformational change causes the Cas10 subunit to generate a linear or cyclic oligonucleotide that activates a nuclease, the activated nuclease cleaving the nucleic acid tether to thereby release the fluorophore from the quencher; and where detecting the one or more products may include detecting a level of fluorescence of the released fluorophore. The tether may include a ribonucleic acid and/or a deoxyribonucleic acid tether.

Contacting the sample with the engineered type III CRISPR-Cas system may include: contacting the sample with a pH-sensitive dye, where the conformational change causes the Cas10 subunit to generate protons that acidifies the solution; and where detecting the occurrence of the conformational change may include detecting acidification of the solution through a change in color of the pH-sensitive dye. Contacting the sample with the engineered type III CRISPR-Cas system may include: contacting the sample with a solution may include a fluorescein dye quenched by metal ions, where the polymerase activity of the first subunit generates pyrophosphates that sequester the metal ions to free the fluorescein dye, the free fluorescein dye binding with the cofactors to generate a fluorescent complex; where detecting the one or more products may include detecting the fluorescent complex.

### BRIEF DESCRIPTION OF THE FIGURES

FIG. 1A illustrates an example of an engineered type III-CRISPR-Cas system of detecting nucleic acid (such as viral RNA) in a sample.

FIG. 1B illustrates an example of identifying a conserved and overrepresented region of the SARS-CoV-2 genome for generating a CRISPR guide sequence.

FIG. 1C illustrates an example of instrumented fluorometric detection.

FIG. 1D illustrates an example of colorimetric detection.

FIG. 1E illustrates an example of visible (naked eye) fluorometric detection.

FIG. 2A illustrates regions of SARS-CoV-2 genome targeted by each of the 10 guides, and schematic of RNA reporter-based assay (right) used to test guides.

FIG. 2B illustrates detection of SARS-CoV-2 IVT RNA, spiked into RNA extracted from patients lacking a SARS-CoV-2 infection, by ten different TtCsm^{Csm3-D34A} complexes (25 nM), via a reporter RNA-based assay illustrated in FIG. 1C and primer sequences.

FIG. 2C illustrates results of direct detection of SARS-CoV-2 genomes.

FIG. 2D illustrates a plot showing that slopes of increasing fluorescence calculated by simple linear regression.

FIG. 3A illustrates a schematic of RT-LAMP-T7-Csm based detection.

FIG. 3B illustrates a plot showing that RT-LAMP-T7-Csm is specific.

FIG. 3C illustrates a plot showing that RT-LAMP-T7-Csm is sensitive.

FIG. 3D illustrates a plot showing kinetics of fluorescence signal increase in T7-Csm reactions.

FIG. 3E illustrates a plot showing results of nasopharyngeal swabs from 56 individuals tested with RT-qPCR (X-axis) and RT-LAMP-T7-Csm (Y-axis).

FIG. 4A illustrates plots of size exclusion chromatography (SEC) profiles of TtCsmWT and TtCsmCsm3-D34A complexes loaded with different crRNA guides.

FIG. 4B illustrates images of SDS-PAGE results of fractions relating to FIG. 4A.

FIG. 4C illustrates a plot of RNA that was isolated from the pooled and concentrated SEC fractions.

FIG. 4D illustrates an SEC profile of TtCsmCsm3-D34A N1 complex.

FIG. 4E illustrates sequence-specific activation of Cas10 estimated for each of the six fractions in FIG. 4D.

FIG. 4F illustrates an SEC profile of TtCsm6 ancillary nuclease purified on a Superdex 200 26/600 size-exclusion column (Cytiva).

FIG. 4G illustrates an SDS-PAGE gel image of purified TtCsm6.

FIG. 5 illustrates a plot of fluorescent signal generation based on cA4-dependent activation of Csm6.

FIG. 6 illustrates colorimetric detection of a specific RNA sequence.

FIGS. 7A and 7B illustrate visible fluorometric detection of a specific RNA sequence.

FIGS. 8A and 8B illustrate rapid and specific detection of SARS-CoV-2 by RT-LAMP-T7-Csm.

FIG. 9 illustrates standard curves for absolute quantification of SARS-CoV-2 titers.

FIG. 10 illustrates a comparison of viral RNA detection using RT-qPCR amplification and viral RNA detection via RT-LAMP-T7 amplification.

FIG. 11 illustrates an example of results of viral RNA detection using RT-qPCR amplification and RT-LAMP-T7 amplification followed by type III-CRISPR-Cas illustrated in FIG. 10.

FIG. 12A illustrates an example of RNase dead mutation in the Csm complex to amplify diagnostic signal.

FIG. 12B illustrates results from a VIRIS detection assay.

FIG. 13A illustrates an example of check-double-check detection of viral RNA in a sample.

FIG. 13B illustrates plots of RT-LAMP-T7 and VIRIS reactions.

### DETAILED DESCRIPTION

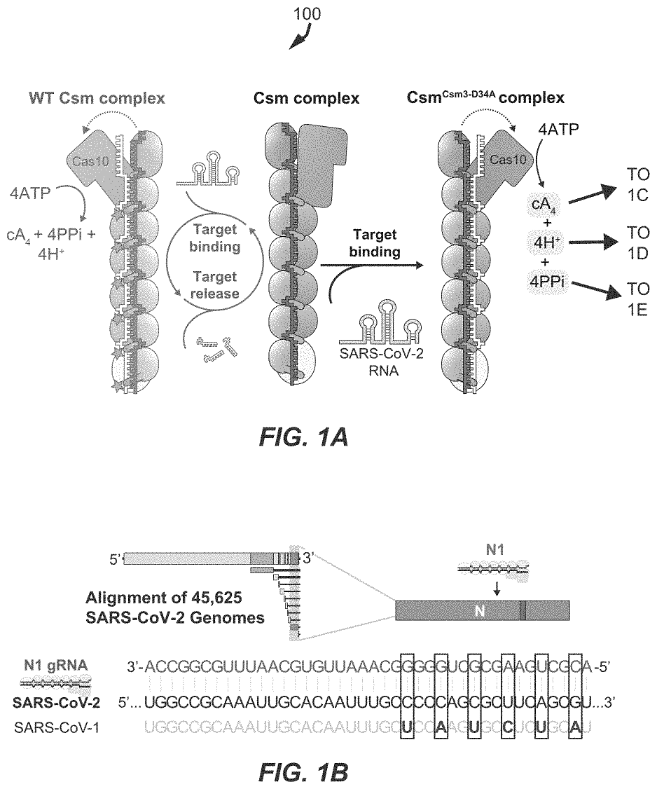

FIG. 1A illustrates an example of an engineered system of detecting nucleic acid in a sample. As used herein, the term “engineered” may refer to a deliberate generation of a system that is otherwise non-naturally occurring. Such engineering may include introducing one or more mutations to a genetic sequence, designing a genetic sequence, combining a set of components such as proteins and detection components where such combination does not occur in nature, and/or otherwise generating a non-naturally occurring system to detect nucleic acid in a sample. The nucleic acid to be detected may be referred to interchangeably herein as a target nucleic acid. The target nucleic acid may be RNA or DNA.

The engineered type III CRISPR-Cas system may be implemented as an assay for testing SARS-CoV-2 virus (or other target nucleic acid in the sample) that can be performed quickly, such as in one hour or less. Nucleic acid recognition by type III systems may trigger Cas10-mediated nuclease activity and/or polymerase activity, which may generate one or more products such as pyrophosphates, protons and cyclic oligonucleotides. The nuclease activity and/or the one or more products of the Cas10-polymerase are detected using colorimetric, visible fluorometric, and/or instrumented fluorometric detection.

The engineered system 100 may include a modified CRISPR complex, detection components, and/or other components. The modified CRISPR complex may include a modified type III CRISPR complex. The modified CRISPR complex may include a CRISPR guide and a plurality of subunits.

The plurality of subunits may include a CRISPR guide, Cas10 subunit, backbone subunits associated with the Csm (such as Csm3) or Cmr systems, and/or other subunits necessary for assembly of the type III surveillance complex as well as the ancillary nucleases (such as Csm6, Can1, Csx). Various examples described herein may describe a CRISPR complex purified from the organism Thermus thermophilus. These examples may further describe the use of protein subunits of T. thermophilus CRISPR complexes. Accordingly, these examples may refer to the subunits as TtCas10, TtCsm3, TtCsm6, and so forth. It should be noted, however, that other protein subunits that perform similar functions may be used as well and/or instead of these examples of subunits.

The CRISPR guide may include a CRISPR guide sequence that is engineered to be complementary to a locus of the nucleic acid. The CRISPR guide sequence may be selected based on one or more conserved regions of the target nucleic acid. For example, FIG. 1B illustrates an example of identifying a conserved region of a target genome for generating a CRISPR guide sequence. Other target nucleic acids from other target organisms (including the subject itself) may be targeted based on FIG. 1B as well. As shown, different genomes of the SARS-CoV-2 virus may be aligned with one another to identify conserved regions. Generally speaking, regions having a greater alignment (fewer base pair differences and longer alignment lengths) than regions having lesser alignment are better candidates for generating a complementary sequence to use as the CRISPR guide sequence.

The CRISPR guide sequence may be designed based on conserved sequence across different samples of the SARS-CoV-2, different strains of the SARS-CoV-2, and/or other samples available for the SARS-CoV-2. Such conserved sequence may be determined based on sequence alignments. A pairwise match may be considered when an alignment quality of the pairwise match is sufficient to determine that aligned portions of two sequences represent a conservation of the nucleotides in the sequences across genomes of SARS-CoV-2 (or other target). The alignment quality may be specified as having a minimum overlap of at least about 1 base, 2 bases, 4 bases, 4 bases, 5 bases, 10 bases, 15 bases, 40 bases, 25 bases, 40 bases, 45 bases, 40 bases, 45 bases, 50 bases, 55 bases, 60 bases, 65 bases, 70 bases, 75 bases, 80 bases, 85 bases, 90 bases, 95 bases, or 100 bases. Alternatively, or additionally, the alignment quality may be based on a minimum alignment identity of at least about 5%, 10%, 15%, 40%, 25%, 40%, 45%, 40%, 45%, 50%, 55%, 60%, 65%, 70%, 75%, 80%, 85%, 90%, 95%, or more. In some cases, a criterion may require at least a 25-nt overlap with at least about 70% identity.

In this example, the sequencing encoding the SARS-CoV-2 nucleocapsid (N) gene was selected to serve as the basis for generating the CRISPR guide sequence. Examples of the CRISPR guide sequence include SEQ ID NO. 1 and SEQ ID NO. 2.

Recently, loop-mediated isothermal amplification (LAMP) (Notomi et al., 2000) has been developed as a sensitive (1-100 copies/μL) point-of-care diagnostic (Dao Thi et al., 2020; Zhang et al., 2020). However, LAMP is prone to generating false positives unless a second sequence-specific technique is used to check the amplified DNA (Dao Thi et al., 2020; Rolando et al., 2020). The type V (Cas12-based) and type VI (Cas13-based) CRISPR systems have been coupled to LAMP or RPA (recombinase polymerase amplification) for sensitive and reliable detection of viral nucleic acids (Broughton et al., 2020; Chen et al., 2018; Gootenberg et al., 2018, 2017; Joung et al., 2020). Following isothermal amplification, the RNA-guided Cas12 or Cas13 proteins bind to the amplified target and trigger a non-sequence specific nuclease activity that cleaves a fluorophore and quencher labelled DNA or RNA (Chen et al., 2018; Gootenberg et al., 2018). Cleavage of the tether results in an increase in fluorescence that can be detected in 45 minutes. While Cas12 and Cas13 detection methods have been optimized over several iterations to be compatible with isothermal amplification of viral RNA, the ultimate goal is to develop CRISPR-based technologies that are sensitive enough to detect the viral RNA directly, without prior amplification. Recently, Fozouni et al reported that the type IV (Cas13a-based) CRISPR systems can be used for amplification-free detection of SARS-CoV-2 RNA in ˜30 minutes and with sensitivity of ˜100 copies/μL (Fozouni et al., 2020).

Like the type VI (Cas13-based) systems, type III systems also target RNA (Hale et al., 2009; Kazlauskiene et al., 2016; Samai et al., 2015; Staals et al., 2014; Tamulaitis et al., 2014). However, type III systems rely on a unique intrinsic signal amplification mechanism (FIG. 1). Here we set out to determine if intrinsic amplification by type III systems could be used to detect viral RNA without prior amplification. The proof-of-concept presented here demonstrates that the Csm complex from Thermus thermophilus (TtCsm) can be programed to specifically recognize the SARS-CoV-2 genome. SARS-CoV-2, but not SARS-CoV-1 or a panel of other respiratory pathogens, activates the Cas10 polymerase, which generates ˜1000 cyclic nucleotides (e.g., cA4) after binding an RNA target (Jia et al., 2019; Kazlauskiene et al., 2017; Niewoehner et al., 2017; Rouillon et al., 2018). Like all polymerases, nucleotide polymerization by Cas10, also generates protons (H⁺) and pyrophosphates (PPi). As disclosed herein, each of these three products can be used, separately or in combination with one another, to detect SARS-CoV-2 RNA using either colorimetric, fluorometric, or both methods simultaneously. The assay can be performed in 1 to 30 minutes depending on the detection method and the concentration of the RNA. When coupled with reverse-transcription LAMP (RT-LAMP) the assay can be performed in less than 30 minutes and has a limit of detection of ˜200 copies/μL.

Results and Discussion

Sequence-Specific Activation of Cas10 Polymerase Yields Three Detectable Products

Sequence-specific recognition of RNA by type III CRISPR systems initiates a signaling Cascade, as illustrated in FIG. 1A. (Kazlauskiene et al., 2017; Niewoehner et al., 2017; Rouillon et al., 2018). RNA binding by the TtCsm complex triggers a conformational change that activates the Palm domain of the Cas10 subunit, which amplifies the RNA binding signal by converting ATP into approximately 1,000 cyclic oligoadenylates (e.g. cA4) (Jia et al., 2019; Kazlauskiene et al., 2017; Niewoehner et al., 2017; Rouillon et al., 2018). We hypothesized that the intrinsic signal amplification unique to type III CRISPR systems would boost the sensitivity of direct RNA detection, while maintaining specificity. To test this hypothesis, we expressed and purified the type III-A CRISPR RNA (crRNA)-guided surveillance complex from Thermus thermophilus (TtCsm) with a guide complementary to the N-gene of SARS-CoV-2 (crRNA_{N1}), an example of which is illustrated in FIG. 1B.

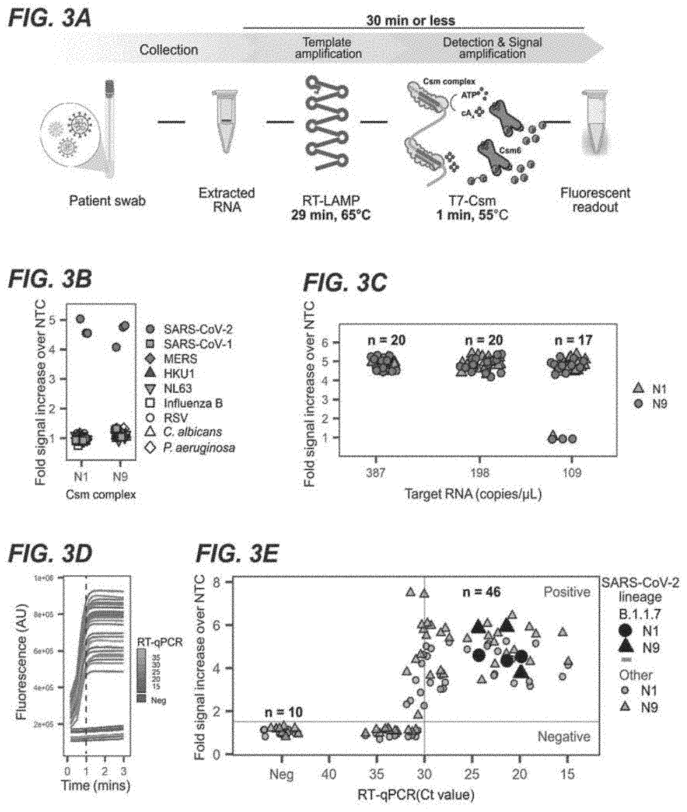

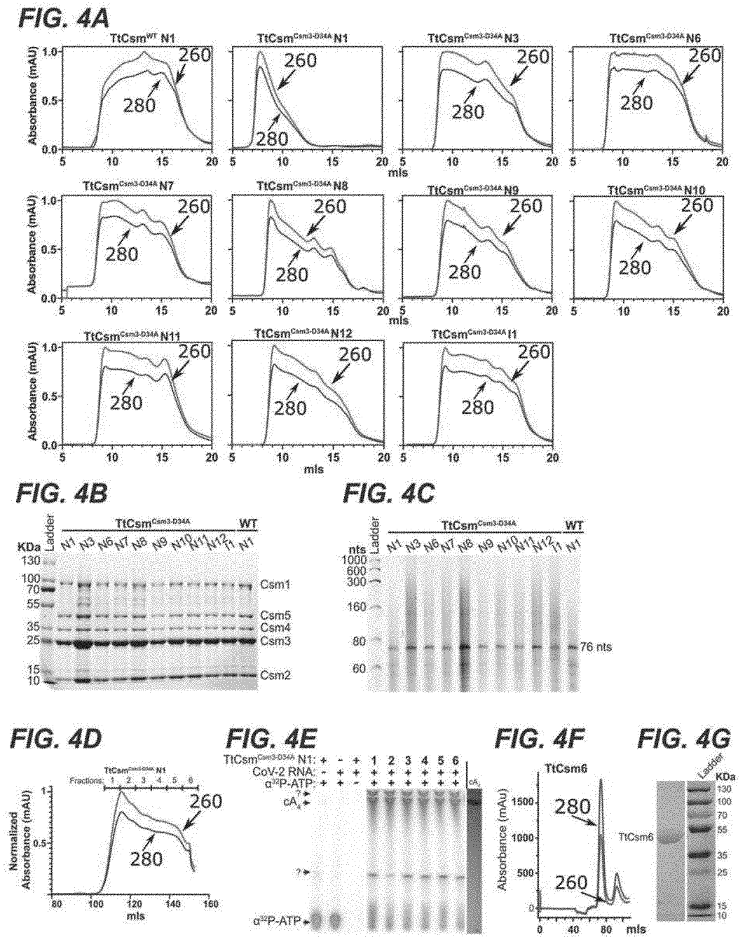

Still referring to FIG. 1A, the Csm3 subunits, which form the “backbone” of the Csm complex, are nucleases that cleave bound target RNA in 6-nt increments (Liu et al., 2017; Samai et al., 2015; Tamulaitis et al., 2014). The cleaved RNA fragments dissociate and the Csm complex returns to the “inactive” state (i.e., no Cas10-polymerase activity) (Nasef et al., 2019; Rouillon et al., 2018). Thus, in the context of an immune response, the RNase activity of Csm3 moderates Cas10 polymerase activity to limit excess nuclease activation that may otherwise kill the cell (Athukoralage et al., 2020; Nasef et al., 2019; Rouillon et al., 2018). However, we reasoned that a Csm3 mutation that prevents target RNA degradation, would have two related benefits as a diagnostic. First, an RNase-dead Csm complex is expected to stay bound to target RNA longer, which would sustain the Cas10 polymerase activity. Second, Csm3-mediated cleavage of the target RNA (e.g., SARS-CoV-2 RNA) would reduce the target RNA concentration over time and thus limit the sensitivity of the assay. Therefore, we mutated residues in the Csm3 subunit responsible for target RNA cleavage (D34A) (Liu et al., 2017; Tamulaitis et al., 2014), and purified the RNase-dead complex (TtCsm^{csm3-D34A}) as illustrated in FIG. 4. To measure the limit of detection (LoD), we added the mutant or wildtype Csm complex to a reaction containing the TtCsm6 nuclease, a fluorescent reporter (i.e., FAM-RNA-Iowa Black FQ), and a titration of RNA corresponding to the N-gene of either SARS-CoV-2 or SARS-CoV-1. An example of fluorometric detection based on a fluorescent report is illustrated in FIG. 1C. An example of Csm-mediated synthesis of cA4 is necessary for activation of the non-sequence specific nuclease Csm6 is illustrated in FIG. 5.

An example of purification of TtCsm complexes and TtCsm6 is illustrated in FIGS. 4A-G and examples of TtCsm guide RNA sequences are provided in the seq listing (SEQ ID NOS. 3 to 12).

FIG. 4A illustrates plots of size exclusion chromatography (SEC) profiles of TtCsmWT and TtCsmCsm3-D34A complexes loaded with different crRNA guides. SEC performed using a Superose 6 Increase 10/300 GL size-exclusion column (Cytiva). Normalized absorbance (mAU) was measured at 260 nm (“260”) and 280 nm (“280”). Fractions 9 up to 16 of each SEC purification were collected, concentrated, and stored at −80° C.

FIG. 4B illustrates images of SDS-PAGE results. Fractions discussed in FIG. 4A were combined, concentrated and run on an SDS-PAGE. All five Csm proteins are present and the intensities of each band correspond with our understanding of the protein stoichiometry of the assembled TtCsm complex.

FIG. 4C illustrates a plot of RNA that was isolated from the pooled and concentrated SEC fractions. Denaturing urea polyacrylamide gel of nucleic acids associated with each TtCsm complex. The full-length crRNA intermediate is expected to be 76 nucleotides (nts) long. FIG. 4D illustrates an SEC profile of TtCsmCsm3-D34A N1 complex. Six successive fractions, representing the entire peak, were collected concentrated and stored separately.

FIG. 4E illustrates sequence-specific activation of Cas10 was estimated for each of the six fractions illustrated in FIG. 4D. 32P-ATP polymerization was measured using thin-layer chromatography (TLC). 500 nM TtCsmCsm3-D34A N1 complex was incubated with 10¹⁰ copies of target RNA, 50 μM ATP and 10 nM α32P-ATP, at 60° C. for 1 hour. Nucleic acids were phenol-chloroform extracted from each reaction and spotted on a silica gel TLC plate coated with fluorescent indicator F254, developed in solvent (0.2 M ammonium bicarbonate pH 9.3, 70% ethanol, 30% water). An unlabeled cA4 standard (Axxora) was run on the same TLC plate, in a parallel lane, and was visualized by illumination with a handheld shortwave (254 nm) UV lamp (Analytik Jena) and a Galaxy S9 phone (Samsung). One of the two major 32P-labelled products generated by target RNA-bound TtCsm complex migrates similarly to the cA4 standard. All TtCsm complexes polymerize similar amounts of α32P-ATP (bottom band) into similar ratios of similarly migrating products (top bands).

FIG. 4F illustrates an SEC profile of TtCsm6 ancillary nuclease purified on a Superdex 200 26/600 size-exclusion column (Cytiva). FIG. 4G illustrates an SDS-PAGE gel image of purified TtCsm6.

Referring to FIG. 5, detection of in vitro transcribed SARS-CoV-2 RNA using the type III surveillance complex TtCsm^{Csm3-D34A} and the ancillary nuclease, TtCsm6. TtCsm^{csm3-D34A}, TtCsm6 and SARS-CoV-2 RNA, but not SARS-CoV-1 RNA, may all be required to trigger cleavage of the fluorescent RNA reporter. Addition of cA4, bypasses the signaling pathway and activates TtCsm6 directly, cleaving the reporter RNA in the absence of SARS-CoV-2 RNA (last lane). These reactions were performed using 10⁸ copies of in vitro transcribed SARS-CoV-1 or -2 RNA spiked into nasopharyngeal swab clinical matrix. Results from three technical replicates are shown. Results presented in the final column (cA4) demonstrate that the fluorescent signal is generated by cA4-dependent activation of Csm6. An example of fluorescent reporter RNAs is illustrated in Table 1.

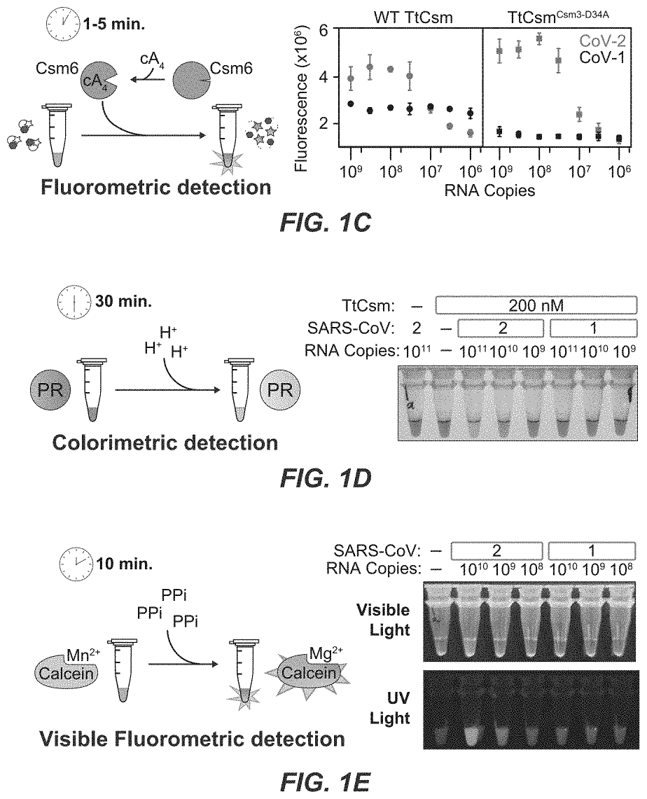

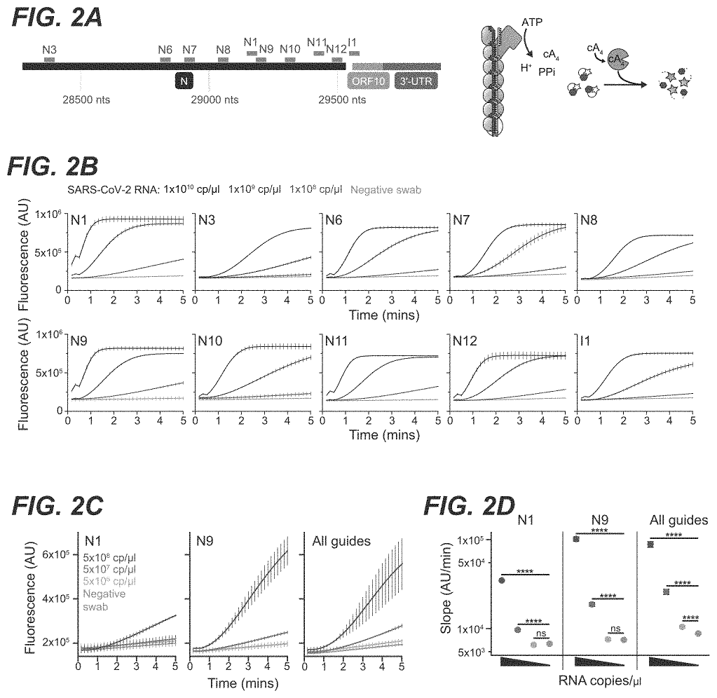

TABLE 1
Examples of fluorescent reporter RNAs.

| Name | Sequence (5’-3’) |
|---|---|
| RNA reporter A | /56-FAM/rCrUrCrUrCrU/3IABkFQ/(FIG. 1) |
| RNA reporter B | /56-FAM/rArUrCrUrUrCrUrUrArU/3IABkFQ/ |
| | (FIGS. 2 and 3) |

Single mismatches in the target RNA have been shown to result in 10-fold lower amounts of cyclic oligoadenylate production by other Csm complexes (Nasef et al., 2019). Using fluorometric detection, both the mutant and the wildtype Csm complex could detect the SARS-CoV-2 RNA at concentrations above 10⁸ copies per reaction, and neither complex cross-reacted with the SARS-CoV-1 RNA at the highest concentrations tested. The RNase-dead TtCsm complex was roughly 3-fold more sensitive than wildtype, with an LoD of ˜10⁷ copies per reaction.

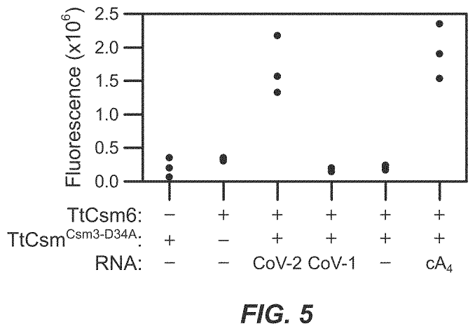

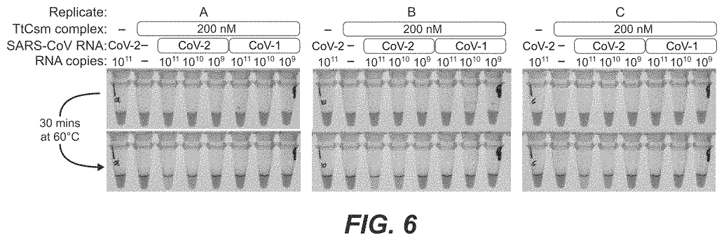

FIGS. 1D and 6 each illustrate colorimetric RNA detection that utilizes a pH change that occurs during nucleotide polymerization. Referring to FIG. 6, TtCsm^{Csm3-D34A} was incubated with either RNA target, in the presence of ATP, for 30 minutes at 60° C. Specific RNA recognition, and associated Cas10-mediated ATP polymerization, causes acidification of the solution, and changes the color of Phenol Red pH indicator from fuchsia, through orange, to yellow. Three technical replicates (A, B and C) are shown. Specific recognition of SARS-CoV-2 by RNase-dead TtCsm complex, activates Cas10. Cas10 polymerizes ATP (Jia et al., 2019; Kazlauskiene et al., 2017; Niewoehner et al., 2017; Rouillon et al., 2018), releasing one proton per incorporated nucleotide. Cas10-generated protons acidify the solution and change the color of a pH indicator (i.e. Phenol Red) from fuchsia through orange (10¹⁰ RNA copies), to yellow (10¹¹ RNA copies). Similarly, we developed a visible fluorometric detection method that relies on the sequestration of metallic ions by pyrophosphate. The metal indicator Calcein is initially quenched by bound Mn²⁺ ions (Tomita et al., 2008). In addition to the cyclic oligoadenylates and protons, Cas10 polymerase generates one pyrophosphate per ATP polymerized. Pyrophosphate forms an insoluble precipitate with Mn²⁺, which unquenches Calcein. Free Calcein is then bound by excess Mg²⁺, forming a fluorescent complex that can be seen by eye or with a UV lamp in less than 10 minutes as illustrated in FIGS. 1E, 7A and 7B. Referring to FIG. 7A, TtCsm^{Csm3-D34A} was incubated with no RNA, a specific (i.e., SARS-CoV-2) or non-specific (SARS-CoV-1) RNA target, in the presence of ATP for 50 minutes at 60° C. The reactions contain an excess of Mg²⁺ relative to Mn²⁺ ions, and the metal indicator Calcein. Calcein preferentially binds Mn²⁺, forming a quenched complex. Specific RNA recognition, and associated Cas10-mediated ATP polymerization, generates pyrophosphate which precipitates with Mg2+ and Mn2+ ions. Excess Mg²⁺ ions bind Calcein to form a highly fluorescent complex that can be seen by eye in visible light or with UV light. Referring to FIG. 7B, kinetics of Calcein fluorescence for corresponding reactions shown in FIG. 7A. 10¹⁰ SARS-CoV-2 RNAs can be detected in 10 minutes, and 10⁹ copies are detected in 40 minutes. No increase in fluorescence is seen beyond the first five minutes for either a no RNA control, or for SARS-CoV-1 RNA containing samples.

Csm-Based Direct Detection of SARS-CoV-2 RNA in Patient Samples

The LoD using crRNA_{N1} is between 10⁷ and 10⁸ copies of IVT RNA per μL, which is insufficient to be clinically relevant. To identify other guides that might outperform or complement the activity of crRNA_{N1} we aligned 45,641 SARS-CoV-2 genomes available from GISAID (Elbe and Buckland-Merrett, 2017). These alignments were used to select guides based on four key criteria. First, each target sequence had to be more than 99% identical among the available SARS-CoV-2 genomes. Second, complementarity between the target and the crRNA was not allowed to extend beyond the spacer sequence (guide), and into repeat derived portions of the crRNA that have been shown to suppress Cas10 activity (Kazlauskiene et al., 2017). Third, we targeted regions of SARS-CoV-2 that were different by at least two-nucleotides in SARS-CoV-1 and MERS-CoV. Fourth, the list of target sequences was pruned to remove guides with similarity to human mRNA sequences, or common oral and respiratory pathogen sequences (E-value <1000). Finally, we focused on target sequences located 3′ of the ORF3a gene, which are present on both the viral genome and on subgenomic RNAs generated during infection. In total, we designed crRNAs targeting 10 different locations on the SARS-CoV-2 genome as illustrated in FIG. 2A. Examples of the guide RNAs are listed as SEQ ID NOS. 3-12. FIG. 2A illustrates regions of SARS-CoV-2 genome targeted by each of the 10 guides, and schematic of RNA reporter-based assay (right) used to test guides.

To determine how each of these guides perform, we measured sequence specific detection of RNA using a fluorometric reporter assay (i.e., FAM-RNA-Iowa Black FQ), results of which are illustrated in FIG. 2B. FIG. 2B illustrates detection of SARS-CoV-2 IVT RNA, spiked into RNA extracted from patients lacking a SARS-CoV-2 infection, by ten different TtCsm^{Csm3-D34A} complexes (25 nM), via a reporter RNA-based assay illustrated in FIG. 1C and primer sequences (SEQ ID NOS. 13-18). Mean and standard deviation of two technical replicates is shown. Most of the crRNAs provide similar sensitivity, however crRNA_{N1} and crRNA_{N9} generated significantly more signal than the next best complex tested (p-value<0.0001). We then tested crRNAN1 and crRNAN9 on RNA isolated from the nasal swab of an infected patient results of which are illustrated in FIG. 2C. FIG. 2C illustrates direct detection of SARS-CoV-2 genomes in RNA extracted from patient samples by 25 nM TtCsm^{Csm3-D34A} N1 or N9, or a mixture of ten different TtCsm^{Csm3-D34A} complexes each at 2.5 nM, via a reporter RNA-based assay. RNA extracted from a patient with a high viral load (5×10⁸ copies/μL as determined by RT-qPCR) was diluted into RNA extracted from patients lacking a SARS-CoV-2 infection. Mean and standard deviation of three technical replicates is shown. Both crRNA_{N1} and crRNA_{N9} guided complexes generate a similar signal for either IVT RNA or RNA isolated from a SARS-CoV-2 positive patient (i.e., 2- to 3-fold increase in signal by 5 minutes, relative to first timepoint). The LoD for crRNA_{N1} and crRNA_{N9} is 10⁷ copies/μL as illustrated in FIG. 2D (p-value<0.0001). Referring to FIG. 2D, slopes of increasing fluorescence, illustrated in FIG. 2C, were calculated by simple linear regression. The calculated slope and ±95% confidence intervals are shown. Positive RNA slopes were compared to the negative swab RNA slope by an F-test: ****p<0.0001, ns=not significantly higher than negative swab RNA control.

Fozouni et al., recently showed that multiplexing Cas13 (i.e., combining multiple guides into a single reaction) improves the sensitivity of SARS CoV-2 detection (Fozouni et al., 2020). We reasoned that similar benefits might be possible for Csm-based detection. To test this idea, we combined 10 of the guides (2.5 nM each) into a single multiplexed reaction. Multiplexing 10 guides improves the sensitivity of TtCsm-mediated detection of SARS-CoV-2 RNA isolated form the nasal swab of a positive patient by approximately 10 times as shown in FIGS. 2C and 2D. However, the sensitivity of direct detection appears to increase additively with the number of TtCsm complexes, which precludes direct detection of RNA at concentration that are clinically relevant.

Testing Clinical Samples for SARS-CoV-2 Using RT-LAMP and T7-Csm.

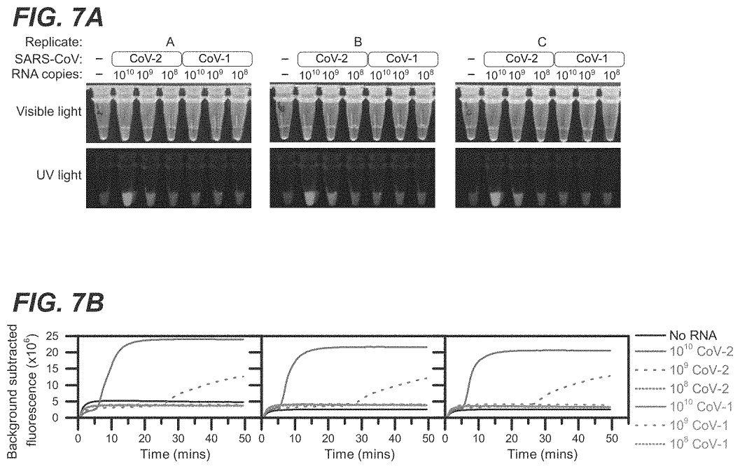

Csm-based detection is currently not sensitive enough to directly detect SARS-CoV-2 in all patients capable of spreading the infection, which requires an LoD of 10³ RNA copies/μL. (La Scola et al., 2020; Larremore et al., 2021; Paltiel et al., 2020; Wölfel et al., 2020). To decrease the LoD of a type III CRISPR-based diagnostic to 10³ RNA copies/μL or lower, we incorporated an upstream nucleic acid amplification technique as illustrated in FIG. 3A. FIG. 3A illustrates a schematic of RT-LAMP-T7-Csm based detection. The viral RNA is reverse transcribed, and resulting DNA is amplified in an RT-LAMP reaction to produce transcription templates for T7 RNA polymerase, in one pot. An aliquot of the RT-LAMP reaction (29 min) is then mixed with the T7-Csm reaction (1 min). First, SARS-CoV-2 genomic RNA is reverse transcribed (RT) into DNA, which is then amplified by LAMP using primers that flank regions of the SARS-CoV-2 genome targeted by crRNA_{N1} and crRNA_{N9}. One of the LAMP primers incorporates a T7 promoter into the amplified DNA, which is then used for in vitro transcription (T7) and detected by TtCsm as illustrated in FIGS. 3A and 8. Examples of primers for RT-LAMP are provided in the sequence listing (SEQ ID NOS. 19-25).

To confirm the specificity of TtCsm-based detection, we tested SARS-CoV-2 alongside a panel of eight other oral and respiratory pathogens, including coronaviruses SARS-CoV-1, Middle East respiratory syndrome coronavirus (MERS-CoV), Human coronavirus HKU1 and Human coronavirus NL63 as illustrated in FIG. 3B. FIG. 3B illustrates a plot showing that RT-LAMP-T7-Csm is specific. Neither crRNA_{N1} or crRNA_{N9} cross-react with other coronaviruses, or other common human oral pathogens or flora. Detection of SARS-CoV-2 by both crRNAs is rapid, 1 min, and robust, 4-5-fold increase in signal over no template control (NTC). Technical replicates are shown in triplicate.

These samples resulted in background signal similar to the no template control (NTC). In contrast, SARS-CoV-2 RNA results in a 4-5-fold increase in signal.

To determine the LoD of RT-LAMP-T7-Csm, we tested 20 replicates of 2-fold serial dilutions ranging from ˜100-400 copies/μL SARS-CoV-2 RNA as illustrated in FIG. 3C. FIG. 3C illustrates a plot showing that RT-LAMP-T7-Csm is sensitive. TtCsm^{csm3-D34A} complexes loaded with either crRNA_{N1} or crRNA_{N9} have LoDs of 198 copies/μL (20/20 replicates).

The LoD of RT-LAMP-T7-Csm is 198 copies/μL SARS-CoV-2 RNA (20/20 replicates), in an assay that relies on a 29-minute RT-LAMP step, followed by a 1-minute T7-Csm fluorometric detection reaction.

FIG. 3D illustrates a plot showing kinetics of fluorescence signal increase in T7-Csm reactions. SARS-CoV-2 positive patient samples are observed to have a ˜2-fold increase in signal over NTC by 10 seconds (crRNA_{N1} median=1.8, crRNA_{N9} median=2.3), which rapidly increases to a 4-5-fold increase in signal over an NTC reaction by 1 minute (crRNA_{N1} median=4.2, crRNA_{N9} median=5.5). A subset of traces is shown for clarity.

To further validate this method, we next tested RNA extracted from 56 nasopharyngeal swab samples taken from patients that had previously been tested using RT-qPCR. Of the 56 samples tested, 46 were positive for SARS-CoV-2 and 10 were negative by RT-qPCR as illustrated in FIG. 3E. FIG. 3E illustrates a plot showing results of nasopharyngeal swabs from 56 individuals tested with RT-qPCR (X-axis) and RT-LAMP-T7-Csm (Y-axis). Swabs with Ct values below 40 for both N1 and N2 CDC diagnostic primers are considered positive for SARS-CoV-2 RNA. RT-LAMP-T7-Csm reliably identifies patient samples with a Ct<30.7 (200-100 RNA copies/μL) as positive for SARS-CoV-2. The B.1.1.7 variant is positively identified by both crRNA_{N1} and crRNA_{N9}. Data is shown as fold change in fluorescence as compared to NTC reaction.

Using two different crRNA guides, we demonstrate that the type III CRISPR system has a specificity (negative predictive agreement) of 100%, as well as a positive predictive agreement of 100% for nasopharyngeal swab samples with 100-200 copies/μL SARS-CoV-2 RNA as determined by RT-qPCR. Whole genome sequencing revealed three of the patient samples used here belonging to the B.1.1.7. lineage. These genome sequences have been deposited in GISAID (Accession IDs: EPI_ISL_1081321, EPI-ISL_1081322, EPI_ISL_1081323) (Elbe and Buckland-Merrett, 2017). Importantly, the B.1.1.7. variants were positively identified by RT-LAMP-T7-Csm with both N1 and N9 crRNA guides (illustrated in FIG. 3E as filled squares for N1 filled diamonds for N9). FIGS. 8A and 8B illustrate rapid and specific detection of SARS-CoV-2 by RT-LAMP-T7-Csm. Raw fluorescence kinetics for the T7-Csm stage of RT-LAMP-T7-Csm detection of SARS-CoV-2 RNA from patient samples with Ct values of 14.9-30.4 (left), 30.7-36.2 (middle), and 40+(right), as detected by the RNase dead TtCsm complex (dTtCsm) loaded with (A) crRNA_{N1} or (B) crRNA_{N9}. Reactions were first incubated in an RT-qPCR machine at 4° C. and fluorescence was measured every 15 seconds for 150 seconds. Fluorescence readings in both positive and negative reactions are low until the T7-Csm reactions are heated to 55° C., upon which fluorescence rapidly increases within 10 seconds and continues to increase for 60 seconds in most samples.

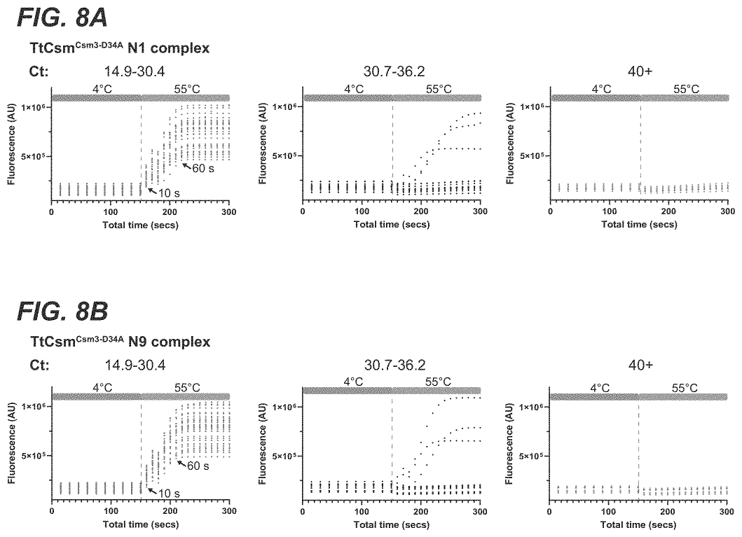

FIG. 9 illustrates standard curves for absolute quantification of SARS-CoV-2 titers. A 10-fold dilution series of SARS-CoV-2 synthetic RNA fragment (RTGM 10169, National Institute of Standards and Technology) containing the Nucleocapsid gene was used. Data was plotted as Cycle Threshold (Ct) on y-axis versus log 10 (copies per ml) on x-axis. The mean Ct of three technical replicates are shown, error bars represent ±1 standard deviation. Trend lines were fit to the data using the geom_smooth function of the ggplot2 R package; linear equations and R2 values are shown. Boundaries corresponding to non-infectious and infectious Ct values refer to observations that patients with viral titers below 10⁶ copies/ml are rarely infectious.

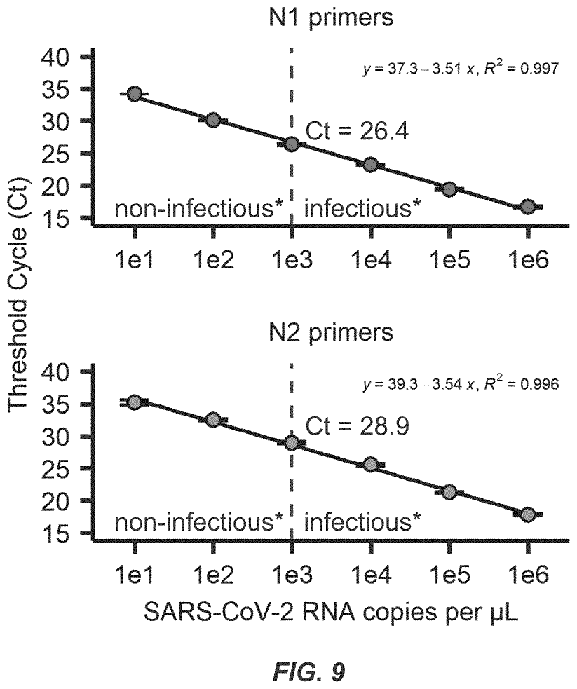

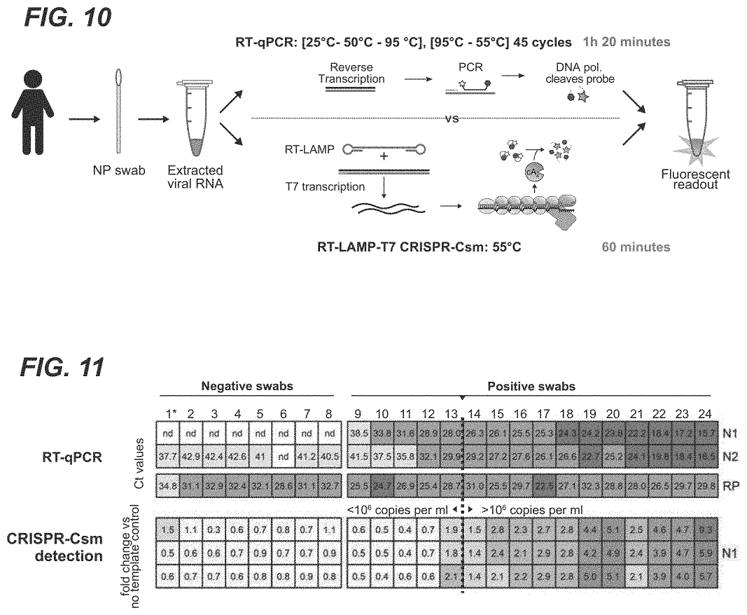

FIG. 11 illustrates an example of results of viral RNA detection using RT-qPCR amplification and RT-LAMP-T7 amplification illustrated in FIG. 10.

In some examples, at least one of the plurality of subunits may be genetically modified. For example, the TtCsm3 subunit may be genetically modified according to the sequence of SEQ ID NO. 27.

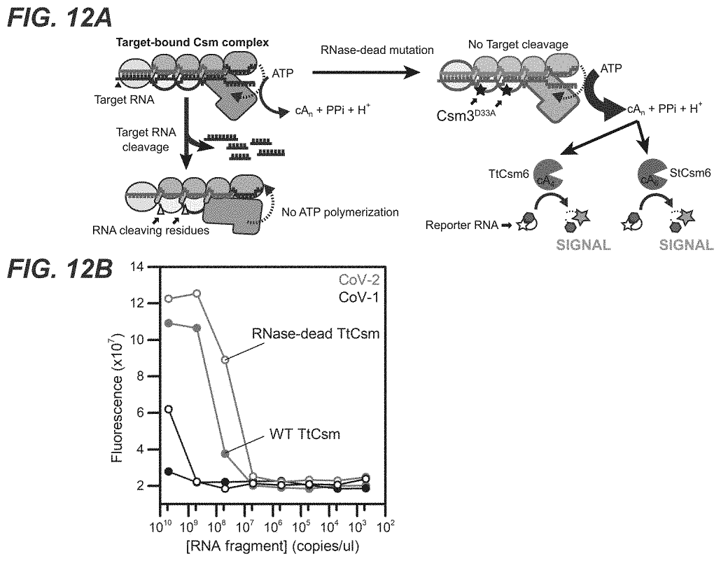

FIG. 12A illustrates an example of RNase dead mutation to amplify diagnostic signal. Wildtype TtCsm complex cleaves bound RNA target over time, decreasing the amount of target RNA and limiting the amount of cyclic oligonucleotides produced per bound RNA target. Mutation of aspartic acid residue number 34 on the Csm3 subunit to alanine (Csm3-D34A; black stars) eliminates the RNase activity of the TtCsm complex, resulting in a complex that no longer turns-over the target RNA. Thus, RNA binding by the RNase dead Csm complex locks the complex into a conformational state that polymerizes NTPs (i.e., “ON” state). More cyclic oligonucleotides are produced for every RNA target bound by a mutant TtCsm complex, as compared to wildtype. Further, a mixture of ancillary nucleases that are activated by different cyclic oligonucleotides (i.e., TtCsm6 activated by cA4; StCsm6 activated by cA6) could be used to convert more of the cyclic nucleotide pool into a fluorescent signal by cleavage of the tether. FIG. 12B illustrates results from a VIRIS detection assay. RNase-dead TtCsm complex binds to SARS-CoV-2 RNA fragment and produces cA4, which activates the ancillary nuclease TtCsm6, which then cleaves a fluorescent reporter RNA, causing an increase in fluorescence. The RNase-dead TtCsm complex allows for more sensitive detection of 5×10⁷ copies of SARS-CoV-2 RNA fragment, as compared to wildtype TtCsm complex. While the D34A mutation in Csm3 eliminates the RNase activity, it may also change the profile of cyclic nucleotides. This change may not activate as many molecules of the ancillary nuclease (TtCsm6) as expected, and this may explain the modest increase in fluorescent signal. A combination of ancillary nucleases that are each activated by different cyclic nucleotides can be used to fully convert the signal generated by TtCsm into cleavage of a reporter RNA. While the species of oligomeric nucleotides may differ, the byproducts of nucleotide polymerization (PPi and protons) will remain unaffected and are expected to increase the sensitivity of detection using pH sensitive indicators.

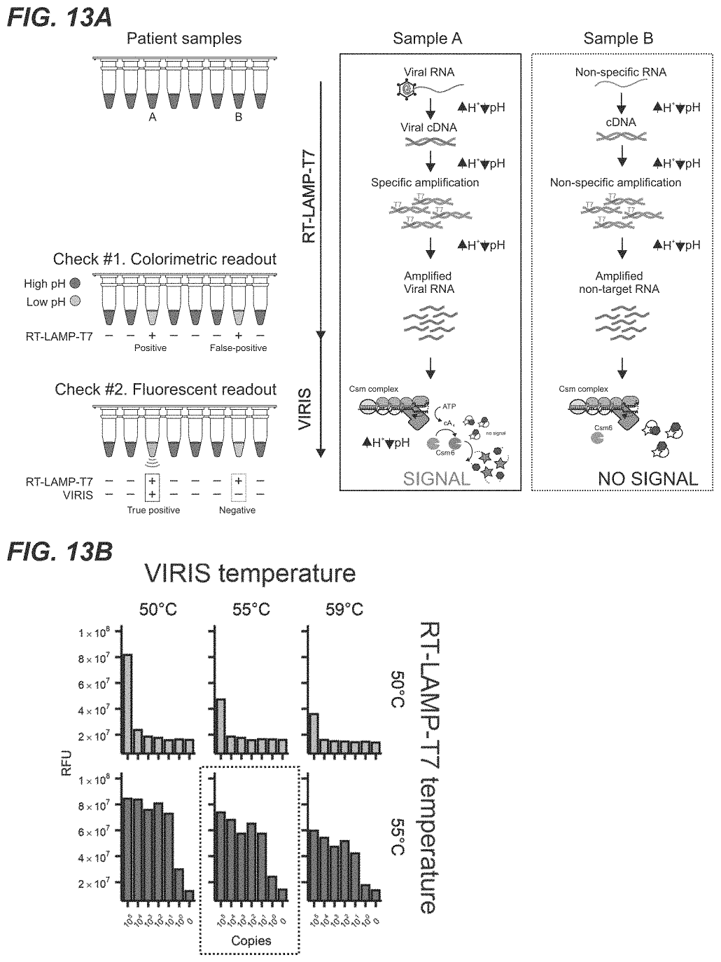

FIG. 13A illustrates an example of check-double-check detection of viral RNA in a sample. RNA is amplified by RT-LAMP-T7 using sequence specific primers (Sample A). RNA is first converted to cDNA by reverse transcriptase. The cDNA is further amplified using Loop-Mediated Isothermal Amplification (LAMP). A promoter for the T7 RNA polymerase is incorporated into amplified cDNA. Hi-T7 polymerase uses the cDNA as a template for transcription of more target (e.g., SARS-CoV-2) RNA. The polymerase activities of RT-LAMP-T7 produce protons (i.e., H+) and thus reduces the pH of the reaction. The change in pH is detected using a pH-sensitive dye (Check #1). The type III crRNA-guided surveillance complex (e.g., Csm complex) is then used to reduce the risk of potential false-positive results due to non-specific polymerization (Sample B). CRISPR RNA (crRNA)-guided binding of type III surveillance complexes to amplified target RNA that is complementary to the crRNA-guide (Sample A) triggers the Cas10 subunit to synthesize a mixture of linear and cyclic oligonucleotides. Sequence-specific activation of the Cas10 polymerase activity generates more protons, which accelerates the decrease in pH and thus accelerates the colorimetric readout in a way that decreases the time to result and adds sequence specificity to the generation of this signal. In addition to generating protons, the Cas10-synthesized oligonucleotides activate previously dormant ancillary nucleases (e.g., Csm6), which cleave an RNA tether connecting a quencher to a fluorophore. Cleavage of the tether liberates the fluorophore, resulting in a fluorescent signal. Positive results of RT-LAMP-T7 are “double-checked” using a fluorimeter to verify a target-specific (e.g., SARS-CoV-2) signal (Check #2). Nonspecifically amplified RNA due to miss-priming during LAMP, could results in a color change (Sample B), resulting in a false positive. The “double-check” is enabled by crRNA-guided binding to a specific RNA, which simultaneously generates protons and cyclic nucleotides. The latter activate ancillary nucleases, which are used to generate a sequence specific fluorescent signal. Samples positive in RT-LAMP-T7 and negative in VIRIS are ruled out as false-positives.

FIG. 13B illustrates plots of RT-LAMP-T7 and VIRIS reactions. A single temperature (55° C., boxed graph) can be used for both RT-LAMP-T7 and VIRIS reactions, and reliably detects 10 copies of SARS-CoV-2 RNA with potential for detection of as low as 1 copy of the virus. One-pot RT-LAMP-T7 reactions with serial dilutions of SARS-CoV-2 genomes were performed at two different temperatures (rows). Subsequent VIRIS detection reactions were performed at three different temperatures (columns) using 5 μL of pre-amplified sample. Signal was measured with fluorescence plate-reader and plotted on y-axis in Relative Fluorescence Units (RFU)

Examples of Methods

Nucleic Acid Preparation

Previously published LAMP primers (Eurofins) were designed to amplify the SARS-CoV-2 N-gene (Broughton et al., 2020). Target SARS-CoV-2 and SARS-CoV-1 RNAs were in vitro transcribed with MEGAscript T7 (Thermo Fisher Scientific) from PCR products generated from pairs of synthesized overlapping DNA oligos or using SARS-CoV-2 genome as a template (SEQ ID NOS. 13-17). Previously designed primer pools (IDT) were used for RT-PCR and sequencing of SARS-CoV-2 genomes (https://artic.network/ncov-2019) (link should omit spaces). Transcribed RNAs were purified by denaturing PAGE. Fluorescent reporter RNA A and fluorescent reporter RNA B purified by RNase-free HPLC (See Table 1) (IDT). Purified genomes of viral, bacterial and fungal pathogens were used as is, or resuspended in 1×TE (10 mM Tris-HCl pH 7.5, 1 mM Ethylenediaminetetraacetic acid (EDTA)) to ˜1×10⁶ genomes/μL. Examples of purified genomic nucleic acids (such as purified genomes of the viral, bacterial and fungal pathogens) are illustrated in Table 2.

| Name | Source |
|---|---|
| SARS-CoV-2 | The National Institute of Standards and |
| | Technology (RGTM 10169) |
| SARS-CoV-1 | American Type Culture Collection |
| | (ATCC) (VR-3280SD) |
| MERS-CoV | ATCC (VR-3248SD) |
| Human coronavirus | ATCC (VR-3262SD) |
| HKU1 | |
| Influenza B | ATCC (VR-1885DQ) |
| Human coronavirus | ATCC (VR-3263SD) |
| NL63 | |
| Human respiratory | ATCC (VR-1580DQ) |
| syncytial virus | |
| Pseudomonas | ATCC (27853D-5) |
| aeruginosa | |
| Candida albicans | ATCC (10231D-5) |

Plasmids

Expression vectors for Thermus thermophilus type III-A Csm1-Csm5 genes, pCDF-5×T7-TtCsm (Liu et al., 2019) were used as a template for site-directed mutagenesis to mutate the Csm3 residue D33 to alanine (D33A) to inactivate Csm3-mediated cleavage of target RNA (pCDF-5×T7-Tt^{CsmCsm3-D34A}) (Liu et al., 2017). The CRISPR array in pACYC-TtCas6-4×crRNA4.5 (Liu et al., 2019) was replaced with a synthetic CRISPR array (GeneArt) containing five repeats and four identical spacers, designed to target the N-gene of SARS-CoV2 (i.e., pACYC-TtCas6-4×gCoV2N1). TtCas6 was PCR amplified from the pACYC-TtCas6-4×crRNA4.5 plasmid and cloned between the NcoI and XhoI sites of pRSF-1b (pRSF-TtCas6). The CARF-HEPN nuclease TtCsm6 was expressed from pC0075 TtCsm6 His6-TwinStrep-SUMO-BsaI (Gootenberg et al., 2018).

Protein Purifications

Expression and purification of the TtCsm complex was performed as previously described with minor modifications (Liu et al., 2019). Briefly, the crRNA plasmid (such as pACYC-TtCas6-4×gCoV2N1) was co-transformed with pRSF-TtCas6 and either pCDF-5×T7-TtCsm or pCDF-5×T7-Tt^{CsmCsm3-D34A} into Escherichia coli BL21(DE3) cells and grown in LB Broth (Lennox) (Thermo Fisher Scientific) at 37° C. to an OD₆₀₀ of 0.5. Cultures were then induced with 0.5 mM IPTG (isopropyl-β-D-thiogalactoside) for expression overnight at 25° C. Cells were pelleted (3,000×g for 25 mins at 4° C.) and lysed via sonication in Lysis buffer (25 mM HEPES pH 7.5, 150 mM KCl, 10 mM imidazole, 1 mM TCEP, 0.01% Triton X-100, 5% glycerol, 1 mM PMSF). Lysate was clarified by centrifugation at 10,000×g for 25 mins at 4° C. The lysate was then heat-treated at 55° C. for 45 minutes and further clarified by centrifugation at 10,000×g for 25 mins at 4° C. His-tagged Csm1 and TtCsm complex were bound to HisTrap HP resin (Cytiva) and washed with Wash buffer (50 mM HEPES pH 7.5, 150 mM KCl, 1 mM TCEP, 5% glycerol, 20 mM imidazole). Protein was eluted in Lysis buffer supplemented with 300 mM imidazole. Eluted protein was concentrated (100 k MWCO Corning Spin-X concentrators) at 4° C. before further purification over HiLoad Superdex 200 26/600 or Superose 6 Increase 10/300 GL size-exclusion columns (Cytiva) in 25 mM HEPES pH 7.5, 150 mM NaCl, 5% glycerol, 1 mM TCEP. Fractions containing the TtCsm complex were pooled, concentrated, aliquoted, flash frozen in liquid nitrogen, and stored at −80° C.

Expression and purification of TtCsm6 was performed as previously described with minor modifications (Gootenberg et al., 2018). pTtCsm6 was transformed into Escherichia coli BL21(DE3) cells and grown in LB Broth (Lennox) (Thermo Fisher Scientific) at 37° C. to an OD₆₀₀ of 0.5. Cultures were then incubated on ice for 1 hour, and then induced with 0.5 mM IPTG for expression overnight at 16° C. Cells were lysed via sonication in TtCsm6 Lysis buffer (20 mM Tris-HCl pH 8, 500 mM NaCl, 1 mM TCEP) and lysate was clarified by centrifugation at 10,000×g for 25 mins at 4° C. The lysate was heat-treated at 55° C. for 45 minutes and clarified by centrifugation at 10,000×g for 25 mins at 4° C. His6-TwinStrep-tagged TtCsm6 was bound to StrepTrap HP resin (Cytiva) and washed in TtCsm6 Lysis buffer. The protein was eluted with TtCsm6 Lysis buffer supplemented with 2.5 mM desthiobiotin and concentrated (10 k MWCO Corning Spin-X concentrators) at 4° C. Affinity tags were removed from TtCsm6 using SUMO protease (100 μL of 2.5 mg/ml protease per 20 mg of TtCsm6 substrate) during dialysis against SUMO digest buffer (30 mM Tris-HCl pH 8, 500 mM NaCl 1 mM DTT, 0.15% Igepal) at 4° C. overnight. Cleaved His6-TwinStrep tag and uncleaved His6-TwinStrep-TtCsm6 were removed by binding to HisTrap HP resin (Cytiva), and the flow-through was concentrated using Corning Spin-X concentrators at 4° C. Finally, TtCsm6 was purified using a HiLoad Superdex 200 26/600 size-exclusion column (Cytiva) in 20 mM Tris-HCl pH 7.5, 1 mM DTT, 400 mM monopotassium glutamate, 5% glycerol. Fractions containing TtCsm6 were pooled, concentrated, aliquoted, flash frozen in liquid nitrogen, and stored at −80° C.

To screen guide RNAs in a high throughput format, ten TtCsm complexes were first crudely purified. 8 mL cultures of E. coli BL21-DE3 cells transformed with pTtCsm and pT7-5×CRISPR-Cas6 were grown at 37° C. and 250 RPM in LB media with selective antibiotics until they reached an OD₆₀₀ reading of 0.4. Protein expression was then induced with the addition of 0.5 mM IPTG to the media, and cells were grown overnight at 16° C. Cells were collected by centrifugation at 4000 RPM, and cell pellets were resuspended in 250 μL of Ni-NTA Equilibration buffer (PBS; 100 mM sodium phosphate, 600 mM sodium chloride), 0.05% Tween™-20 Detergent, 30 mM imidazole; pH 8.0). Resuspended cells were sonicated twice for twenty seconds, then clarified by centrifugation at 15,000 rpm for 20 minutes at −4° C. to remove cellular debris. The lysate was then heat-treated at 55° C. for 45 minutes, and re-clarified by centrifugation at 15,000 rpm, for 30 mins at 4° C. TtCsm was then purified using HisPur Ni-NTA magnetic beads (ThermoFisher) according to the manufacturers recommendations, but with modified wash (25 mM HEPES pH 7.5, 150 mM NaCl, 0.05% Tween-20, 1 mM TCEP) and equilibration (25 mM HEPES pH 7.5, 150 mM NaCl, 1 mM TCEP) and elution buffers (25 mM HEPES pH 7.5, 150 mM NaCl, 1 mM TCEP, 300 mM Imidazole). TtCsm complex concentration was quantified on a Nanodrop (ThermoFisher).

Type III CRISPR-Based RNA Detection

Fluorescent CRISPR-Csm Based Detection

For experiments shown in FIG. 1C, RNA was extracted from nasopharyngeal swabs derived from patients that tested negative for SARS-CoV-2 as determined by RT-qPCR. This RNA was used as is or spiked with in vitro transcribed SARS-CoV-2 or SARS-CoV-1 RNA. These RNA samples were mixed with 250 μM ATP, 500 nM fluorescent reporter RNA A, 500 nM TtCsm complex, and 2500 nM of TtCsm6 in reaction buffer (20 mM Tris-HCl pH 7.9, 200 mM Monopotassium glutamate, 10 mM Ammonium sulphate, 5 mM Magnesium sulphate and 1 mM TCEP (tris(2-carboxyethyl)phosphine)) in a 30 μL reaction. Reactions were incubated at 60° C. (CRISPR-Csm alone), and fluorescence was measured over time in an ABI 7500 Fast Real-Time PCR System (Applied Biosystems), using the manufacturers default filter settings for FAM dye. Fluorescence measurements at an incubation time of 45 minutes are reported.

For experiments shown in FIG. 2C, an RNA detection mixture was made containing either 25 nM TtCsm^{CsmCsm3-D34A} N1, or 25 nM TtCsm^{CsmCsm3-D34A}, or 2.5 nM each of ten complexes (TtCsm^{CsmCsm3-D34A} N1, N3, N6, N7, N8, N9, N10, N11, N12 and I1), mixed with 250 μM ATP, 150 nM fluorescent reporter RNA B, 300 nM TtCsm6, in reaction buffer (20 mM Tris-HCl pH 7.8, 250 mM Monopotassium glutamate, 10 mM Ammonium sulphate, 5 mM Magnesium sulphate and 1 mM TCEP). 3 μL of RNA extracted from a patient nasopharyngeal swab with high SARS-CoV-2 viral load (˜5×10⁸ copies/μL) was added to 27 μL of the above RNA detection mixture. Alternatively, RNA from this positive patient sample was first diluted 10- or 100-fold into RNA extracted from a patient negative for SARS-CoV-2 (CT>40), and 3 μL of these dilutions was added to 27 μL of the above RNA detection mixture. Reactions were incubated at 60° C. and fluorescence was measured every 10 seconds for up to 20 minutes, in a QuantStudio 3 Real-Time PCR system (ThermoFisher), using the manufacturers default filter settings for FAM dye.

Colorimetric CRISPR-Csm Based Detection

TtCsm^{Csm3-D34A} stocks were buffer exchanged into a low buffering capacity buffer (0.5 mM Tris-HCl pH 8.8, 50 mM Potassium chloride, 10 mM Ammonium sulphate, 8 mM Magnesium sulphate) using Microspin G25 columns (Cytiva) as per the manufacturer's instructions. TE buffer (10 mM Tris-HCl pH 7.5, 1 mM EDTA) or in vitro transcribed SARS-CoV-2 or SARS-CoV-1 RNA were incubated with 200 nM TtCsm^{Csm3-D34A} in 1× WarmStart Colorimetric LAMP Master Mix (NEB), supplemented with an additional 1 mM ATP, in a 25 μL reaction. The volume of buffer-exchanged TtCsm used contributed approximately 40 μM Tris-HCl pH 8.8 buffer to the final reaction. Reactions were assembled on ice and imaged on an LED tracing pad with a Galaxy S9 phone (Samsung). Then reactions were incubated at 60° C. for 30 minutes, rapidly cooled, and imaged again.

Visible Fluorometric CRISPR-Csm Based Detection

TE buffer or in vitro transcribed SARS-CoV-2 or SARS-CoV-1 RNA were incubated with 500 nM TtCsm^{Csm3-D34A} in reaction buffer (20 mM Tris-HCl pH 8.8, 100 mM Potassium chloride, 10 mM Ammonium sulphate, 6 mM Magnesium sulphate, 0.5 mM Manganese chloride, 1 mM TCEP, 1 mM ATP and 25 μM Calcein), in a 30 μL reaction. Reactions were incubated at 60° C., and fluorescence was measured over time in an ABI 7500 Fast Real-Time PCR System (Applied Biosystems), using the manufacturers default filter settings for FAM dye. After incubating at 60° C. for 50 minutes, the same reactions were then imaged under visible light, and under UV light (365 nm) with a Galaxy S9 phone (Samsung). To screen guide RNAs in a high throughput format (FIG. 2B), 200 nM crude purified TtCsm complex was incubated with 10¹² copies of IVT SARS-CoV-2 RNA in the above buffers and fluorescence was recorded in a ABI 7500 Fast Real-Time PCR System (Applied Biosystems) machine as above.

RT-LAMP-T7-Csm

Isothermal amplification of nucleic acids in swab samples was performed by RT-LAMP. In brief, 25 μL reactions contained 8 units (U) of WarmStart Bst 2.0 (NEB), and 7.5 U of WarmStart RTx Reverse Transriptase (NEB), 1.4 mM dNTPs, LAMP primers, 25 U of Murine RNase Inhibitor (NEB) in reaction buffer (20 mM Tris-HCl pH 7.8, 8 mM Magnesium sulfate, 10 mM Ammonium sulfate, 50 mM potassium chloride, 0.1% Tween-20). LAMP primers designed to amplify the SARS-CoV-2 N-gene (Broughton et al., 2020), were added at an optimized final concentration of 0.2 μM F3 and B3, 0.4 μM LoopF and LoopB, 1.6 μM BIP, 0.53 μM FIP, and 1.07 μM of T7-FIP (such as the primers for RT-LAMP: SEQ ID NOS. 19-25). The T7-FIP primer consists of a T7 promoter fused to the 5′ end of the FIP primer, and allows for the generation of T7 transcription templates during the second step of T7-Csm reaction. RT-LAMP reactions were performed using 5 μL of input RNA at 65° C. for 29 minutes. 3 μL of RT-LAMP reactions were mixed with 27 μL of a modified T7-Csm fluorescent detection reaction containing 0.5 mM rNTPs, 300 nM TtCsm6, 150 nM fluorescent reporter RNA B, and 20 nM of either TtCsm^{Csm3-D34A} N1 or N9, in reaction buffer (40 mM Tris-HCl pH 7.5, 4 mM Magnesium chloride, 50 mM Sodium chloride, 2 mM spermidine, 1 mM DTT). Reactions were incubated at 55° C. for up to 20 min and fluorescence kinetics was monitored in a QuantStudio 3 Real-Time PCR system (ThermoFisher) as described above.

LoD standards were prepared by diluting SARS-CoV-2 RNA into RNA extracted from COVID-19-negative patient nasopharyngeal swabs. Concentrations were determined with RT-qPCR using a standard curve generated from 10-fold dilution series)(1×10⁶-1×10⁰ of IVT fragment.

Human Clinical Sample Collection and Preparation

Nasopharyngeal swabs from patients that either tested negative or positive for SARS-CoV-2 were collected in viral transport media. RNA was extracted from all patient samples using QIAamp Viral RNA Mini Kit (Qiagen).

RT-qPCR

RT-qPCR was performed using two primers pairs (N1 and N2) and probes from the 2019-nCoV CDC EUA Kit (IDT #10006606). SARS-CoV-2 in RNA-extracted, nasopharyngeal patient samples was detected and quantified using one-step RT-qPCR in ABI 7500 Fast Real-Time PCR System according to CDC guidelines and protocols (https://www.fda.gov/media/134922/download) (link should omit spaces). In brief, 20 μL reactions included 8.5 μL of Nuclease-free Water, 1.5 μL of Primer and Probe mix (IDT, 10006713), 5 μL of TaqPath 1-Step RT-qPCR Master Mix (ThermoFisher, A15299) and 5 μL of the template. Nuclease-free water was used as negative template control (NTC). Amplification was performed as follows: 25° C. for 2 min, 50° C. for 15 min, 95° C. for 2 min followed by 45 cycles of 95° C. for 3 s and 55° C. for 30 s. To quantify viral genome copy numbers in the samples, standard curves for N1 and N2 were generated using a dilution series of a SARS-CoV-2 synthetic RNA fragment (RTGM 10169, National Institute of Standards and Technology) spanning N gene with concentrations ranging from 10 to 10⁶ copies per μL. Three technical replicates were performed at each dilution. The NTC showed no amplification throughout the 45 cycles of qPCR.

Bioinformatic Design of TtCsm crRNA Guides Targeting SARS-CoV-2

An alignment of 45,641 SARS-CoV-2 genomes was downloaded front the GISAID database (Global Initiative for Sharing All Influenza Data; GISAID.org) (link should omit spaces) on 6-23-2020 (Elbe and Buckland-Merrett, 2017; Katoh and Standley, 2013). The alignment was scanned for conservation with a 40-nucleotide sliding window, and 40-nucleotide segments with strong conservation were saved for downstream analysis. Next, four nucleotides flanking the above 40-nucleotide candidate viral target sequences were checked for base pairing to the first four nucleotides of the prospective 5′-crRNA handle (underlined; 5′-AUUGCGAC-3′), only candidates lacking handle complementarity were considered further. Candidate sites with less than two mismatches to SARS-CoV (NC_004718.3) and MERS-CoV (NC_019843.3) in the first 18 nucleotides of the target sequence were discarded. Next, candidate crRNAs targeting the above sites were screened for potential cross-reactivity with human mRNAs and a list of human pathogens and common respiratory flora downloaded from the FDA's Emergency Use Authorization requirements (downloaded on 7-29-2020) using BLAST (−evalue 1000). The remaining 6,229 crRNA sequences were then sorted by genomic location and only guides that were located 3′ of the SARS-CoV-2 ORF3a gene (positions 25,393 to 29,903) were considered further. Finally, 76 guides were selected from the remaining pool that had the greatest conservation amongst SARS-CoV-2. sequences and the largest number of mismatches to SARS and MERS-CoV sequences.

Sequencing of SARS-CoV-2 RNA Isolated from Patient Samples

SARS-CoV-2 genomic RNA isolated from patient samples was sequenced as previously described (Nemudryi et al., 2021). In brief, 10 μL of SARS-CoV-2 genomic RNA extracted from nasopharyngeal patient swabs was first reverse transcribed with SuperScript IV (ThermoFisher) according to the manufacturer's instructions. The ARTIC Network protocol was followed to generate a sequence amplicon library covering the whole SARS-CoV-2 genome on Oxford Nanopore using a ligation sequencing kit (Oxford Nanopore, SQK-LSK109) (https://artic.network/ncov-2019) (Grubaugh et al., 2019; Tyson et al., 2020) (link should omit spaces). Two multiplex PCR reactions were performed with primer pools described in the ARTIC nCoV-2019 V3 Panel (such as primers to generate an amplicon library for SARS-CoV-2 whole genome sequencing: SEQ ID NOS. 29-246), amplified with Q5 DNA Polymerase (NEB). The two resulting amplicon pools for each patient sample were then combined and used for library preparation. Samples were end repaired (NEB, E7546) and then barcoded using Native Barcoding Expansion Kits (Oxford Nanopore, EXP-NBD104 and EXP-NBD114). Barcoded samples were pooled together and then Nanopore adaptors were ligated.

The multiplexed library was loaded onto the MinION flowcell, and a total of 0.3 Gb of raw sequencing data was collected per patient sample. Raw Nanopore reads were base-called in high-accuracy mode (Oxford Nanopore, MinKNOW), and further analyzed using the ARTIC bioinformatic pipeline for COVID-19 (https://artic.network/ncov-2019) (link should omit spaces). Consensus sequences were uploaded to GISAID (https://www.gisaid.org/) (link should omit spaces), IDs: EPI_ISL_1081321, EPI-ISL_1081322, EPI_ISL_1081323 (Elbe and Buckland-Merrett, 2017). These three SARS-CoV-2 genome sequences were identified as members of the B.1.1.7 lineage by an automated lineage assigner (Rambaut et al., 2020) (https://github.com/hCoV-2019/pangolin) (link should omit spaces).

Statistical Analyses

All experiments were performed in triplicate or duplicate and error is reported as ±1 standard deviation. The merged datasets of replicates of fluorescence kinetics of direct Csm-based detection of SARS-CoV-2 RNA in patient samples was fit to a simple linear regression, in Prism 9 (Graphpad). The fitted slopes of SARS-CoV-2 RNA-containing patient samples were compared pairwise to the negative swab RNA control by an F-test, ****p<0.0001.

### Sequences

TABLE 3
Examples of sequences. SEQ ID NOS. 29-138
belong to primer pool nCoV-2019_1 and SEQ ID NOS. 139-246
belong to primer pool nCoV-2019_2.

| SEQ | | |
|---|---|---|
| ID NO. | Sequence | Description |
| 1 | AUUGCGACACGCUGAAGCGCUGGG | RNA sequence for example |
| | GGCAAAUUGUGCAAUUUGCGGCCA | CRISPR guide. RNA sequences are |
| | GUUGCAAGGGAUUGAGCCCCGUAA | shown 5′ to 3′. Bases in italics |
| | GGGG | correspond to portions of the |
| | | CRISPR repeat. Bases that are |
| | | underlined correspond to the |
| | | CRISPR spacer-that is |
| | | complementary to the SARS-CoV-2 |
| | | N gene. |
| 2 | AUUGCGACACGCUGAAGCGCUGGG | RNA sequence for example |
| | GGCAAAUUGUGCAAUUUGCGGCCA | CRISPR guide. RNA sequences are |
| | GUUGCAAG | shown 5′ to 3′. Bases in italics |
| | | correspond to portions of the |
| | | CRISPR repeat. Bases that are |
| | | underlined correspond to the |
| | | CRISPR spacer-that is |
| | | complementary to the SARS-CoV-2 |
| | | N gene. |
| 3 | AUUGCGACACGCUGAAGCGCUGGG | N1 (76 nt guide) |
| | GGCAAAUUGUGCAAUUUGCGGCCA | |
| | GUUGCAAGGGAUUGAGCCCCGUAA | |
| | GGGG | |
| 4 | AUUGCGACGGCCGACGUUGUUUUG | N3 (76 nt guide) |
| | AUCGCGCCCCACUGCGUUCUCCAU | |
| | GUUGCAAGGGAUUGAGCCCCGUAA | |
| | GGGG | |
| 5 | AUUGCGACGUUGCGACUACGUGAU | N6 (76 nt guide) |
| | GAGGAACGAGAAGAGGCUUGACU | |
| | GGUUGCAAGGGAUUGAGCCCCGUA | |
| | AGGGG | |
| 6 | AUUGCGACAGCAGCAGCAAAGCAA | N7 (76 nt guide) |
| | GAGCAGCAUCACCGCCAUUGCCAG | |
| | GUUGCAAGGGAUUGAGCCCCGUAA | |
| | GGGG | |
| 7 | AUUGCGACAUGCUUUAGUGGCAG | N8 (76 nt guide) |
| | UACGUUUUUGCCGAGGCUUCUUAG | |
| | AGUUGCAAGGGAUUGAGCCCCGUA | |
| | AGGGG | |
| 8 | AUUGCGACUUCCGAAGAACGCUGA | N9 (76 nt guide) |
| | AGCGCUGGGGGCAAAUUGUGCAA | |
| | UGUUGCAAGGGAUUGAGCCCCGUA | |
| | AGGGG | |
| 9 | AUUGCGACAUUCAGCAAAAUGACU | N10 (76 nt guide) |
| | UGAUCUUUGAAAUUUGGAUCUUU | |
| | GGUUGCAAGGGAUUGAGCCCCGUA | |
| | AGGGG | |
| 10 | AUUGCGACCAGUUUGCUGUUUCUU | N11 (76 nt guide) |
| | CUGUCUCUGCGGUAAGGCUUGAGU | |
| | GUUGCAAGGGAUUGAGCCCCGUAA | |
| | GGGG | |
| 11 | AUUGCGACGUCAGCACUGCUCAUG | N12 (76 nt guide) |
| | GAUUGUUGCAAUUGUUUGGAGAA | |
| | AGUUGCAAGGGAUUGAGCCCCGUA | |
| | AGGGG | |
| 12 | AUUGCGACAAAAGCGAAAACGUU | I1 (76 nt guide) |
| | UAUAUAGCCCAUCUGCCUUGUGUG | |
| | GGUUGCAAGGGAUUGAGCCCCGUA | |
| | AGGGG | |
| 13 | GATAATACGACTCACTATAGGGAA | SARS-CoV-2 N1 T7 template |
| | CTGATTACAAACATTGGCCGCAAAT | Forward Primer |
| | TGCACAATT | |
| 14 | GCGCGACATTCCGAAGAACGCTGA | SARS-CoV-2 N1 T7 template |
| | AGCGCTGGGGGCAAATTGTGCAAT | Reverse Primer |
| | TTGCGGCC | |
| 15 | GATAATACGACTCACTATAGGGAA | SARS-CoV-1 N1 T7 template |
| | CTGATTACAAACATTGGCCGCAAAT | Forward Primer |
| | TGCACAATT | |
| 16 | GCGTGACATTCCAAAGAATGCAGA | SARS-CoV-1 N1 T7 template |
| | GGCACTTGGAGCAAATTGTGCAATT | Reverse Primer |
| | TGCGGCC | |
| 17 | CTAGAGCTCGATAATACGACTCACT | SARS-CoV-2 target all ten |
| | ATAGGGCGTGTTGTTTTAGATTTCA | complexes Forward Primer |
| | TCTAAACG | |
| 18 | ATCCTGCAGGCACACTGATTAAAG | SARS-CoV-2 target all ten |
| | ATTGCTATGTG | complexes Reverse Primer |
| 19 | GCTGCTGAGGCTTCTAAG | RT-LAMP Primer F3 |
| 20 | GCGTCAATATGCTTATTCAGC | RT-LAMP Primer B3 |
| 21 | TCAGCGTTCTTCGGAATGTCGCTGT | RT-LAMP Primer BIP |
| | GTAGGTCAACCACG | |
| 22 | GCGGCCAATGTTTGTAATCAGTAGA | RT-LAMP Primer FIP |
| | CGTGGTCCAGAACAA | |
| 23 | CCTTGTCTGATTAGTTCCTGGT | RT-LAMP Primer Loop Forward |
| 24 | TGGCATGGAAGTCACACC | RT-LAMP Primer Loop Reverse |
| 25 | TAATACGACTCACTATAGGGAGAC | RT-LAMP Primer T7-FIP |
| | GTGGTCCAGAACAA | |
| 26 | MKLKKVIRIRSVLLAKTGLRIGMSRD | Amino Acid sequence of Wildtype |
| | QMAIGDLDNPVVRNPLTDEPYIPGSS | TtCsm3 sequence |
| | LKGKLRYLLEWSLGGDYILKAKERQ | |
| | VYASPDPKDPVARIFGLAPENDERSL | |
| | AVARERGPTRLLVRDAYLTEDAKEA | |
| | LERTSARGGLYTEIKQEVFIPRLGGN | |
| | ANPRTTERVPAGARFRVEMTYRVLD | |
| | DLDEEYFGKYLLRALELLELDGLGG | |
| | HISRGYGQVYFLHPERLTEDQEGWPL | |
| | KERLKVEEVVL | |
| 27 | MKLKKVIRIRSVLLAKTGLRIGMSRDQMAIG | Amino Acid sequence of Mutant |
| | DLANPVVRNPLTDEPYIPGSSLKGKLRYLLE | TtCsm3 sequence (showing amino |
| | WSLGGDYILKAKERQVYASPDPKDPVARIFG | acid mutation in bold, underline) |
| | LAPENDERSLAVARERGPTRLLVRDAYLTED | |
| | AKEALERTSARGGLYTEIKQEVFIPRLGGNA | |
| | NPRTTERVPAGARFRVEMTYRVLDDLDEEY | |
| | FGKYLLRALELLELDGLGGHISRGYGQVYFL | |
| | HPERLTEDQEGWPLKERLKVEEVVL | |
| 28 | TAATACGACTCACTATAGGGagacgtg | The T7 promoter sequence is in |
| | gtccagaacaa | CAPS in this primer. The |
| | | lowerCase bases are the same as |
| | | those found in the 3′ end of the |
| | | Forward Inner Primer. |
| 29 | ACCAACCAACTTTCGATCTCTTGT | nCoV-2019_1_LEFT |
| 30 | CATCTTTAAGATGTTGACGTGCCTC | nCoV-2019_1_RIGHT |
| 31 | CGGTAATAAAGGAGCTGGTGGC | nCoV-2019_3_LEFT |
| 32 | AAGGTGTCTGCAATTCATAGCTCT | nCoV-2019_3_RIGHT |
| 33 | TGGTGAAACTTCATGGCAGACG | nCoV-2019_5_LEFT |
| 34 | ATTGATGTTGACTTTCTCTTTTTGGA | nCoV-2019_5_RIGHT |
| | GT | |
| 35 | ATCAGAGGCTGCTCGTGTTGTA | nCoV-2019_7_LEFT |
| 36 | CATTTGCATCAGAGGCTGCTCG | nCoV-2019_7_LEFT_alt0 |
| 37 | TGCACAGGTGACAATTTGTCCA | nCoV-2019_7_RIGHT |
| 38 | AGGTGACAATTTGTCCACCGAC | nCoV-2019_7_RIGHT_alt5 |
| 39 | TCCCACAGAAGTGTTAACAGAGGA | nCoV-2019_9_LEFT |
| 40 | TTCCCACAGAAGTGTTAACAGAGG | nCoV-2019_9_LEFT_alt4 |
| 41 | ATGACAGCATCTGCCACAACAC | nCoV-2019_9_RIGHT |
| 42 | GACAGCATCTGCCACAACACAG | nCoV-2019_9_RIGHT_alt2 |
| 43 | GGAATTTGGTGCCACTTCTGCT | nCoV-2019_11_LEFT |
| 44 | TCATCAGATTCAACTTGCATGGCA | nCoV-2019_11_RIGHT |
| 45 | TCGCACAAATGTCTACTTAGCTGT | nCoV-2019_13_LEFT |
| 46 | ACCACAGCAGTTAAAACACCCT | nCoV-2019_13_RIGHT |
| 47 | ACAGTGCTTAAAAAGTGTAAAAGT | nCoV-2019_15_LEFT |
| | GCC | |
| 48 | AGTGCTTAAAAAGTGTAAAAGTGC | nCoV-2019_15_LEFT_alt1 |
| | CT | |
| 49 | AACAGAAACTGTAGCTGGCACT | nCoV-2019_15_RIGHT |
| 50 | ACTGTAGCTGGCACTTTGAGAGA | nCoV-2019_15_RIGHT_alt3 |
| 51 | CTTCTTTCTTTGAGAGAAGTGAGGA | nCoV-2019_17_LEFT |
| | CT | |
| 52 | TTTGTTGGAGTGTTAACAATGCAGT | nCoV-2019_17_RIGHT |
| 53 | GCTGTTATGTACATGGGCACACT | nCoV-2019_19_LEFT |
| 54 | TGTCCAACTTAGGGTCAATTTCTGT | nCoV-2019_19_RIGHT |
| 55 | TGGCTATTGATTATAAACACTACAC | nCoV-2019_21_LEFT |
| | ACCC | |
| 56 | GGCTATTGATTATAAACACTACACA | nCoV-2019_21_LEFT_alt2 |
| | CCCT | |
| 57 | TAGATCTGTGTGGCCAACCTCT | nCoV-2019_21_RIGHT |
| 58 | GATCTGTGTGGCCAACCTCTTC | nCoV-2019_21_RIGHT_alt0 |
| 59 | ACAACTACTAACATAGTTACACGGT | nCoV-2019_23_LEFT |
| | GT | |
| 60 | ACCAGTACAGTAGGTTGCAATAGT | nCoV-2019_23_RIGHT |
| | G | |
| 61 | GCAATTGTTTTTCAGCTATTTTGCA | nCoV-2019_25_LEFT |
| | GT | |
| 62 | ACTGTAGTGACAAGTCTCTCGCA | nCoV-2019_25_RIGHT |
| 63 | ACTACAGTCAGCTTATGTGTCAACC | nCoV-2019_27_LEFT |
| 64 | AATACAAGCACCAAGGTCACGG | nCoV-2019_27_RIGHT |
| 65 | ACTTGTGTTCCTTTTTGTTGCTGC | nCoV-2019_29_LEFT |
| 66 | AGTGTACTCTATAAGTTTTGATGGT | nCoV-2019_29_RIGHT |
| | GTGT | |
| 67 | TTCTGAGTACTGTAGGCACGGC | nCoV-2019_31_LEFT |
| 68 | ACAGAATAAACACCAGGTAAGAAT | nCoV-2019_31_RIGHT |
| | GAGT | |
| 69 | ACTTTTGAAGAAGCTGCGCTGT | nCoV-2019_33_LEFT |
| 70 | TGGACAGTAAACTACGTCATCAAG | nCoV-2019_33_RIGHT |
| | C | |
| 71 | TGTTCGCATTCAACCAGGACAG | nCoV-2019_35_LEFT |
| 72 | ACTTCATAGCCACAAGGTTAAAGTC | nCoV-2019_35_RIGHT |
| | A | |
| 73 | ACACACCACTGGTTGTTACTCAC | nCoV-2019_37_LEFT |
| 74 | GTCCACACTCTCCTAGCACCAT | nCoV-2019_37_RIGHT |
| 75 | AGTATTGCCCTATTTTCTTCATAAC | nCoV-2019_39_LEFT |
| | TGGT | |
| 76 | TGTAACTGGACACATTGAGCCC | nCoV-2019_39_RIGHT |
| 77 | GTTCCCTTCCATCATATGCAGCT | nCoV-2019_41_LEFT |
| 78 | TGGTATGACAACCATTAGTTTGGCT | nCoV-2019_41_RIGHT |
| 79 | TACGACAGATGTCTTGTGCTGC | nCoV-2019_43_LEFT |
| 80 | AGCAGCATCTACAGCAAAAGCA | nCoV-2019_43_RIGHT |
| 81 | TACCTACAACTTGTGCTAATGACCC | nCoV-2019_45_LEFT |
| 82 | AGTATGTACAAATACCTACAACTTG | nCoV-2019_45_LEFT_alt2 |
| | TGCT | |
| 83 | AAATTGTTTCTTCATGTTGGTAGTT | nCoV-2019_45_RIGHT |
| | AGAGA | |
| 84 | TTCATGTTGGTAGTTAGAGAAAGTG | nCoV-2019_45_RIGHT_alT7 |
| | TGTC | |
| 85 | AGGACTGGTATGATTTTGTAGAAA | nCoV-2019_47_LEFT |
| | ACCC | |
| 86 | AATAACGGTCAAAGAGTTTTAACCT | nCoV-2019_47_RIGHT |
| | CTC | |
| 87 | AGGAATTACTTGTGTATGCTGCTGA | nCoV-2019_49_LEFT |
| 88 | TGACGATGACTTGGTTAGCATTAAT | nCoV-2019_49_RIGHT |
| | ACA | |
| 89 | TCAATAGCCGCCACTAGAGGAG | nCoV-2019_51_LEFT |
| 90 | AGTGCATTAACATTGGCCGTGA | nCoV-2019_51_RIGHT |
| 91 | AGCAAAATGTTGGACTGAGACTGA | nCoV-2019_53_LEFT |
| 92 | AGCCTCATAAAACTCAGGTTCCC | nCoV-2019_53_RIGHT |
| 93 | ACTCAACTTTACTTAGGAGGTATGA | nCoV-2019_55_LEFT |
| | GCT | |
| 94 | GGTGTACTCTCCTATTTGTACTTTA | nCoV-2019_55_RIGHT |
| | CTGT | |
| 95 | ATTCTACACTCCAGGGACCACC | nCoV-2019_57_LEFT |
| 96 | GTAATTGAGCAGGGTCGCCAAT | nCoV-2019_57_RIGHT |
| 97 | TCACGCATGATGTTTCATCTGCA | nCoV-2019_59_LEFT |
| 98 | AAGAGTCCTGTTACATTTTCAGCTT | nCoV-2019_59_RIGHT |
| | G | |
| 99 | TGTTTATCACCCGCGAAGAAGC | nCoV-2019_61_LEFT |
| 100 | ATCACATAGACAACAGGTGCGC | nCoV-2019_61_RIGHT |
| 101 | TGTTAAGCGTGTTGACTGGACT | nCoV-2019_63_LEFT |
| 102 | ACAAACTGCCACCATCACAACC | nCoV-2019_63_RIGHT |
| 103 | GCTGGCTTTAGCTTGTGGGTTT | nCoV-2019_65_LEFT |
| 104 | TGTCAGTCATAGAACAAACACCAA | nCoV-2019_65_RIGHT |
| | TAGT | |
| 105 | GTTGTCCAACAATTACCTGAAACTT | nCoV-2019_67_LEFT |
| | ACT | |
| 106 | CAACCTTAGAAACTACAGATAAAT | nCoV-2019_67_RIGHT |
| | CTTGGG | |
| 107 | TGTCGCAAAATATACTCAACTGTGT | nCoV-2019_69_LEFT |
| | CA | |
| 108 | TCTTTATAGCCACGGAACCTCCA | nCoV-2019_69_RIGHT |
| 109 | ACAAATCCAATTCAGTTGTCTTCCT | nCoV-2019_71_LEFT |
| | ATTC | |
| 110 | TGGAAAAGAAAGGTAAGAACAAGT | nCoV-2019_71_RIGHT |
| | CCT | |
| 111 | CAATTTTGTAATGATCCATTTTTGG | nCoV-2019_73_LEFT |
| | GTGT | |
| 112 | CACCAGCTGTCCAACCTGAAGA | nCoV-2019_73_RIGHT |
| 113 | AGAGTCCAACCAACAGAATCTATT | nCoV-2019_75_LEFT |
| | GT | |
| 114 | ACCACCAACCTTAGAATCAAGATT | nCoV-2019_75_RIGHT |
| | GT | |
| 115 | CCAGCAACTGTTTGTGGACCTA | nCoV-2019_77_LEFT |
| 116 | CAGCCCCTATTAAACAGCCTGC | nCoV-2019_77_RIGHT |
| 117 | GTGGTGATTCAACTGAATGCAGC | nCoV-2019_79_LEFT |
| 118 | CATTTCATCTGTGAGCAAAGGTGG | nCoV-2019_79_RIGHT |
| 119 | GCACTTGGAAAACTTCAAGATGTG | nCoV-2019_81_LEFT |
| | G | |
| 120 | GTGAAGTTCTTTTCTTGTGCAGGG | nCoV-2019_81_RIGHT |
| 121 | TCCTTTGCAACCTGAATTAGACTCA | nCoV-2019_83_LEFT |
| 122 | TTTGACTCCTTTGAGCACTGGC | nCoV-2019_83_RIGHT |
| 123 | ACTAGCACTCTCCAAGGGTGTT | nCoV-2019_85_LEFT |
| 124 | ACACAGTCTTTTACTCCAGATTCCC | nCoV-2019_85_RIGHT |
| 125 | CGACTACTAGCGTGCCTTTGTA | nCoV-2019_87_LEFT |
| 126 | ACTAGGTTCCATTGTTCAAGGAGC | nCoV-2019_87_RIGHT |
| 127 | GTACGCGTTCCATGTGGTCATT | nCoV-2019_89_LEFT |
| 128 | CGCGTTCCATGTGGTCATTCAA | nCoV-2019_89_LEFT_alt2 |
| 129 | ACCTGAAAGTCAACGAGATGAAAC | nCoV-2019_89_RIGHT |
| | A | |
| 130 | ACGAGATGAAACATCTGTTGTCACT | nCoV-2019_89_RIGHT_alt4 |
| 131 | TCACTACCAAGAGTGTGTTAGAGGT | nCoV-2019_91_LEFT |
| 132 | TTCAAGTGAGAACCAAAAGATAAT | nCoV-2019_91_RIGHT |
| | AAGCA | |
| 133 | TGAGGCTGGTTCTAAATCACCCA | nCoV-2019_93_LEFT |
| 134 | AGGTCTTCCTTGCCATGTTGAG | nCoV-2019_93_RIGHT |
| 135 | TGAGGGAGCCTTGAATACACCA | nCoV-2019_95_LEFT |
| 136 | CAGTACGTTTTTGCCGAGGCTT | nCoV-2019_95_RIGHT |
| 137 | TGGATGACAAAGATCCAAATTTCA | nCoV-2019_97_LEFT |
| | AAGA | |
| 138 | ACACACTGATTAAAGATTGCTATGT | nCoV-2019_97_RIGHT |
| | GAG | |
| 139 | CTGTTTTACAGGTTCGCGACGT | nCoV-2019_2_LEFT |
| 140 | TAAGGATCAGTGCCAAGCTCGT | nCoV-2019_2_RIGHT |
| 141 | GGTGTATACTGCTGCCGTGAAC | nCoV-2019_4_LEFT |
| 142 | CACAAGTAGTGGCACCTTCTTTAGT | nCoV-2019_4_RIGHT |
| 143 | GGTGTTGTTGGAGAAGGTTCCG | nCoV-2019_6_LEFT |
| 144 | TAGCGGCCTTCTGTAAAACACG | nCoV-2019_6_RIGHT |
| 145 | AGAGTTTCTTAGAGACGGTTGGGA | nCoV-2019_8_LEFT |
| 146 | GCTTCAACAGCTTCACTAGTAGGT | nCoV-2019_8_RIGHT |
| 147 | TGAGAAGTGCTCTGCCTATACAGT | nCoV-2019_10_LEFT |
| 148 | TCATCTAACCAATCTTCTTCTTGCTC | nCoV-2019_10_RIGHT |
| | T | |
| 149 | AAACATGGAGGAGGTGTTGCAG | nCoV-2019_12_LEFT |
| 150 | TTCACTCTTCATTTCCAAAAAGCTT | nCoV-2019_12_RIGHT |
| | GA | |
| 151 | CATCCAGATTCTGCCACTCTTGT | nCoV-2019_14_LEFT |
| 152 | TGGCAATCTTCATCCAGATTCTGC | nCoV-2019_14_LEFT_alt4 |
| 153 | AGTTTCCACACAGACAGGCATT | nCoV-2019_14_RIGHT |
| 154 | TGCGTGTTTCTTCTGCATGTGC | nCoV-2019_14_RIGHT_alt2 |
| 155 | AATTTGGAAGAAGCTGCTCGGT | nCoV-2019_16_LEFT |
| 156 | CACAACTTGCGTGTGGAGGTTA | nCoV-2019_16_RIGHT |
| 157 | TGGAAATACCCACAAGTTAATGGTT | nCoV-2019_18_LEFT |
| | TAAC | |
| 158 | ACTTCTATTAAATGGGCAGATAACA | nCoV-2019_18_LEFT_alt2 |
| | ACTGT | |
| 159 | AGCTTGTTTACCACACGTACAAGG | nCoV-2019_18_RIGHT |
| 160 | GCTTGTTTACCACACGTACAAGG | nCoV-2019_18_RIGHT_alt1 |
| 161 | ACAAAGAAAACAGTTACACAACAA | nCoV-2019_20_LEFT |
| | CCA | |
| 162 | ACGTGGCTTTATTAGTTGCATTGTT | nCoV-2019_20_RIGHT |
| 163 | ACTACCGAAGTTGTAGGAGACATT | nCoV-2019_22_LEFT |
| | ATACT | |
| 164 | ACAGTATTCTTTGCTATAGTAGTCG | nCoV-2019_22_RIGHT |
| | GC | |
| 165 | AGGCATGCCTTCTTACTGTACTG | nCoV-2019_24_LEFT |
| 166 | ACATTCTAACCATAGCTGAAATCGG | nCoV-2019_24_RIGHT |
| | G | |
| 167 | TTGTGATACATTCTGTGCTGGTAGT | nCoV-2019_26_LEFT |
| 168 | TCCGCACTATCACCAACATCAG | nCoV-2019_26_RIGHT |
| 169 | ACATAGAAGTTACTGGCGATAGTT | nCoV-2019_28_LEFT |
| | GT | |
| 170 | TGTTTAGACATGACATGAACAGGT | nCoV-2019_28_RIGHT |
| | GT | |
| 171 | GCACAACTAATGGTGACTTTTTGCA | nCoV-2019_30_LEFT |
| 172 | ACCACTAGTAGATACACAAACACC | nCoV-2019_30_RIGHT |
| | AG | |
| 173 | TGGTGAATACAGTCATGTAGTTGCC | nCoV-2019_32_LEFT |
| 174 | AGCACATCACTACGCAACTTTAGA | nCoV-2019_32_RIGHT |
| 175 | TCCCATCTGGTAAAGTTGAGGGT | nCoV-2019_34_LEFT |
| 176 | AGTGAAATTGGGCCTCATAGCA | nCoV-2019_34_RIGHT |
| 177 | TTAGCTTGGTTGTACGCTGCTG | nCoV-2019_36_LEFT |
| 178 | GAACAAAGACCATTGAGTACTCTG | nCoV-2019_36_RIGHT |
| | GA | |
| 179 | ACTGTGTTATGTATGCATCAGCTGT | nCoV-2019_38_LEFT |
| 180 | CACCAAGAGTCAGTCTAAAGTAGC | nCoV-2019_38_RIGHT |
| | G | |
| 181 | TGCACATCAGTAGTCTTACTCTCAG | nCoV-2019_40_LEFT |
| | T | |
| 182 | CATGGCTGCATCACGGTCAAAT | nCoV-2019_40_RIGHT |
| 183 | TGCAAGAGATGGTTGTGTTCCC | nCoV-2019_42_LEFT |
| 184 | CCTACCTCCCTTTGTTGTGTTGT | nCoV-2019_42_RIGHT |
| 185 | TGCCACAGTACGTCTACAAGCT | nCoV-2019_44_LEFT |
| 186 | CCACAGTACGTCTACAAGCTGG | nCoV-2019_44_LEFT_alt3 |
| 187 | AACCTTTCCACATACCGCAGAC | nCoV-2019_44_RIGHT |
| 188 | CGCAGACGGTACAGACTGTGTT | nCoV-2019_44_RIGHT_alt0 |
| 189 | TGTCGCTTCCAAGAAAAGGACG | nCoV-2019_46_LEFT |
| 190 | CGCTTCCAAGAAAAGGACGAAGA | nCoV-2019_46_LEFT_alt1 |
| 191 | CACGTTCACCTAAGTTGGCGTA | nCoV-2019_46_RIGHT |
| 192 | CACGTTCACCTAAGTTGGCGTAT | nCoV-2019_46_RIGHT_alt2 |
| 193 | TGTTGACACTGACTTAACAAAGCCT | nCoV-2019_48_LEFT |
| 194 | TAGATTACCAGAAGCAGCGTGC | nCoV-2019_48_RIGHT |
| 195 | GTTGATAAGTACTTTGATTGTTACG | nCoV-2019_50_LEFT |
| | ATGGT | |
| 196 | TAACATGTTGTGCCAACCACCA | nCoV-2019_50_RIGHT |
| 197 | CATCAGGAGATGCCACAACTGC | nCoV-2019_52_LEFT |
| 198 | GTTGAGAGCAAAATTCATGAGGTC | nCoV-2019_52_RIGHT |
| | C | |
| 199 | TGAGTTAACAGGACACATGTTAGA | nCoV-2019_54_LEFT |
| | CA | |
| 200 | AACCAAAAACTTGTCCATTAGCAC | nCoV-2019_54_RIGHT |
| | A | |
| 201 | ACCTAGACCACCACTTAACCGA | nCoV-2019_56_LEFT |
| 202 | ACACTATGCGAGCAGAAGGGTA | nCoV-2019_56_RIGHT |
| 203 | TGATTTGAGTGTTGTCAATGCCAGA | nCoV-2019_58_LEFT |
| 204 | CTTTTCTCCAAGCAGGGTTACGT | nCoV-2019_58_RIGHT |
| 205 | TGATAGAGACCTTTATGACAAGTTG | nCoV-2019_60_LEFT |
| | CA | |
| 206 | GGTACCAACAGCTTCTCTAGTAGC | nCoV-2019_60_RIGHT |
| 207 | GGCACATGGCTTTGAGTTGACA | nCoV-2019_62_LEFT |
| 208 | GTTGAACCTTTCTACAAGCCGC | nCoV-2019_62_RIGHT |
| 209 | TCGATAGATATCCTGCTAATTCCAT | nCoV-2019_64_LEFT |
| | TGT | |
| 210 | AGTCTTGTAAAAGTGTTCCAGAGGT | nCoV-2019_64_RIGHT |
| 211 | GGGTGTGGACATTGCTGCTAAT | nCoV-2019_66_LEFT |
| 212 | TCAATTTCCATTTGACTCCTGGGT | nCoV-2019_66_RIGHT |
| 213 | ACAGGTTCATCTAAGTGTGTGTGT | nCoV-2019_68_LEFT |
| 214 | CTCCTTTATCAGAACCAGCACCA | nCoV-2019_68_RIGHT |
| 215 | ACAAAAGAAAATGACTCTAAAGAG | nCoV-2019_70_LEFT |
| | GGTTT | |
| 216 | TGACCTTCTTTTAAAGACATAACAG | nCoV-2019_70_RIGHT |
| | CAG | |
| 217 | ACACGTGGTGTTTATTACCCTGAC | nCoV-2019_72_LEFT |
| 218 | ACTCTGAACTCACTTTCCATCCAAC | nCoV-2019_72_RIGHT |
| 219 | ACATCACTAGGTTTCAAACTTTACT | nCoV-2019_74_LEFT |
| | TGC | |
| 220 | GCAACACAGTTGCTGATTCTCTTC | nCoV-2019_74_RIGHT |
| 221 | AGGGCAAACTGGAAAGATTGCT | nCoV-2019_76_LEFT |
| 222 | GGGCAAACTGGAAAGATTGCTGA | nCoV-2019_76_LEFT_alt3 |
| 223 | ACACCTGTGCCTGTTAAACCAT | nCoV-2019_76_RIGHT |
| 224 | ACCTGTGCCTGTTAAACCATTGA | nCoV-2019_76_RIGHT_alt0 |
| 225 | CAACTTACTCCTACTTGGCGTGT | nCoV-2019_78_LEFT |
| 226 | TGTGTACAAAAACTGCCATATTGCA | nCoV-2019_78_RIGHT |
| 227 | TTGCCTTGGTGATATTGCTGCT | nCoV-2019_80_LEFT |
| 228 | TGGAGCTAAGTTGTTTAACAAGCG | nCoV-2019_80_RIGHT |
| 229 | GGGCTATCATCTTATGTCCTTCCCT | nCoV-2019_82_LEFT |
| 230 | TGCCAGAGATGTCACCTAAATCAA | nCoV-2019_82_RIGHT |
| 231 | TGCTGTAGTTGTCTCAAGGGCT | nCoV-2019_84_LEFT |
| 232 | AGGTGTGAGTAAACTGTTACAAAC | nCoV-2019_84_RIGHT |
| | AAC | |
| 233 | TCAGGTGATGGCACAACAAGTC | nCoV-2019_86_LEFT |
| 234 | ACGAAAGCAAGAAAAAGAAGTACG | nCoV-2019_86_RIGHT |
| | C | |
| 235 | CCATGGCAGATTCCAACGGTAC | nCoV-2019_88_LEFT |
| 236 | TGGTCAGAATAGTGCCATGGAGT | nCoV-2019_88_RIGHT |
| 237 | ACACAGACCATTCCAGTAGCAGT | nCoV-2019_90_LEFT |
| 238 | TGAAATGGTGAATTGCCCTCGT | nCoV-2019_90_RIGHT |
| 239 | TTTGTGCTTTTTAGCCTTTCTGCT | nCoV-2019_92_LEFT |
| 240 | AGGTTCCTGGCAATTAATTGTAAAA | nCoV-2019_92_RIGHT |
| | GG | |
| 241 | GGCCCCAAGGTTTACCCAATAA | nCoV-2019_94_LEFT |
| 242 | TTTGGCAATGTTGTTCCTTGAGG | nCoV-2019_94_RIGHT |
| 243 | GCCAACAACAACAAGGCCAAAC | nCoV-2019_96_LEFT |
| 244 | TAGGCTCTGTTGGTGGGAATGT | nCoV-2019_96_RIGHT |
| 245 | AACAATTGCAACAATCCATGAGCA | nCoV-2019_98_LEFT |
| 246 | TTCTCCTAAGAAGCTATTAAAATCA | nCoV-2019_98_RIGHT |
| | CATGG | |

A subject may refer to an animal, such as a mammalian species (preferably human) or avian (e.g., bird) species, or other organism, such as a plant. More specifically, a subject can be a vertebrate, e.g., a mammal such as a mouse, a primate, a simian or a human. Animals include farm animals, sport animals, and pets. A subject can be a healthy individual, an individual that has symptoms or signs or is suspected of having a disease or a predisposition to the disease, or an individual that is in need of therapy or suspected of needing therapy.

A genetic modification or mutation in the context of an engineered system may refer to an alteration, variant or polymorphism in a nucleic acid that may result in altered or disabled functionality of a corresponding protein. Such alteration, variant or polymorphism can be with respect to a reference genome, the subject or other individual. Variations include one or more single nucleotide variations (SNVs), insertions, deletions, repeats, small insertions, small deletions, small repeats, structural variant junctions, variable length tandem repeats, and/or flanking sequences, CNVs, transversions, gene fusions and other rearrangements may also be considered forms of genetic variation. A variation can be a base change, insertion, deletion, repeat, copy number variation, transversion, or a combination thereof.

A “polynucleotide”, “nucleic acid”, “nucleic acid molecule”, or “oligonucleotide” may each refer to a polymer of nucleosides (including deoxyribonucleosides, ribonucleosides, or analogs thereof) joined by inter-nucleosidic linkages. Typically, a polynucleotide comprises at least three nucleosides. Oligonucleotides often range in size from a few monomeric units, e.g. 3-4, to hundreds of monomeric units. Whenever a polynucleotide is represented by a sequence of letters, such as “ATGCCTG,” it will be understood that the nucleotides are in 5′→3′ order from left to right and that “A” denotes deoxyadenosine, “C” denotes deoxycytidine, “G” denotes deoxyguanosine, and “T” denotes thymidine, unless otherwise noted. The letters A, C, G, and T (or “U” denoting Uracil in RNA) may be used to refer to the bases themselves, to nucleosides, or to nucleotides comprising the bases, as is standard in the art.

A “polynucleotide”, “nucleic acid”, “nucleic acid molecule”, or “oligonucleotide” may each refer to a polymer of nucleosides (including deoxyribonucleosides, ribonucleosides, or analogs thereof) joined by inter-nucleosidic linkages. Typically, a polynucleotide comprises at least three nucleosides. Oligonucleotides often range in size from a few monomeric units, e.g. 3-4, to hundreds of monomeric units. Whenever a polynucleotide is represented by a sequence of letters, such as “ATGCCTG,” it will be understood that the nucleotides are in 5′→3′ order from left to right and that “A” denotes deoxyadenosine, “C” denotes deoxycytidine, “G” denotes deoxyguanosine, and “T” denotes thymidine, unless otherwise noted. The letters A, C, G, and T (or “U” denoting Uracil in RNA) may be used to refer to the bases themselves, to nucleosides, or

### REFERENCES

Athukoralage J S, Graham S, Rouillon C, Grüschow S, Czekster C M, White M F. 2020. The dynamic interplay of host and viral enzymes in type III CRISPR-mediated cyclic nucleotide signalling. Elife 9:1-16. doi:10.7554/eLife.55852
Broughton J P, Deng X, Yu G, Fasching C L, Servellita V, Singh J, Miao X, Streithorst J A, Granados A, Sotomayor-Gonzalez A, Zorn K, Gopez A, Hsu E, Gu W, Miller S, Pan C, Guevara H, Wadford D A, Chen J S, Chiu C Y. 2020. CRISPR-Cas12-based detection of SARS-CoV-2. Nat Biotechnol. doi:10.1038/s41587-020-0513-4
Chen J S, Ma E, Harrington L B, Da Costa M, Tian X, Palefsky J M, DouDNA J A. 2018. CRISPR-Cas12a target binding unleashes indiscriminate single-stranded DNase activity. Science (80-) 360:436-439. doi:10.1126/science.aar6245
Dao Thi V L, Herbst K, Boerner K, Meurer M, Kremer L P, Kirrmaier D, Freistaedter A, Papagiannidis D, Galmozzi C, Stanifer M L, Boulant S, Klein S, Chlanda P, Khalid D, Miranda I B, Schnitzler P, Kräusslich H-G, Knop M, Anders S. 2020. A colorimetric RT-LAMP assay and LAMP-sequencing for detecting SARS-CoV-2 RNA in clinical samples. Sci Transl Med 7075:1-20. doi:10.1126/scitranslmed.abc7075
Elbe S, Buckland-Merrett G. 2017. Data, disease and diplomacy: GISAID's innovative contribution to global health. Glob Challenges 1:33-46. doi:10.1002/gch2.1018
Elmore J R, Sheppard N F, Ramia N, Deighan T, Li H, Terns R M, Terns M P. 2016. Bipartite recognition of target RNAs activates DNA cleavage by the Type III-B CRISPR-Cas system. Genes Dev 30:447-459. doi:10.1101/gad.272153.115
Fozouni P, Son S, Díaz de Leon Derby M, Knott G J, Gray C N, D'Ambrosio M V., Zhao C, Switz N A, Kumar G R, Stephens S I, Boehm D, Tsou C-L, Shu J, Bhuiya A, Armstrong M, Harris A R, Chen P-Y, Osterloh J M, Meyer-Franke A, Joehnk B, Walcott K, Sil A, Langelier C, Pollard K S, Crawford E D, Puschnik A S, Phelps M, Kistler A, DeRisi J L, DouDNA J A, Fletcher D A, Ott M. 2020. Amplification-free detection of SARS-CoV-2 with CRISPR-Cas13a and mobile phone microscopy. Cell 1-11. doi:10.1016/j.cell.2020.12.001
Gootenberg J S, Abudayyeh 00, Kellner M J, Joung J, Collins J J, Zhang F. 2018. Multiplexed and portable nucleic acid detection platform with Cas13, Cas12a and Csm6. Science (80-) 360:439-444. doi:10.1126/science.aaq0179
Gootenberg J S, Abudayyeh 00, Lee J W, Essletzbichler P, Dy A J, Joung J, Verdine V, Donghia N, Daringer N M, Freije C A, Myhrvold C, Bhattacharyya R P, Livny J, Regev A, Koonin E V., Hung D T, Sabeti P C, Collins J J, Zhang F. 2017. Nucleic acid detection with CRISPR-Cas13a/C2c2. Science (80-) 356.
Grubaugh N D, Gangavarapu K, Quick J, Matteson N L, De Jesus J G, Main B J, Tan A L, Paul L M, Brackney D E, Grewal S, Gurfield N, Van Rompay K K A, Isern S, Michael S F, Coffey L L, Loman N J, Andersen K G. 2019. An amplicon-based sequencing framework for accurately measuring intrahost virus diversity using PrimalSeq and iVar. Genome Biol 20:8. doi:10.1186/s13059-018-1618-7
Hale C R, Zhao P, Olson S, Duff M O, Graveley B R, Wells L, Terns R M, Terns M P. 2009. RNA-Guided RNA Cleavage by a CRISPR RNA-Cas Protein Complex. Cell 139:945-956. doi:10.1016/j.cell.2009.07.040
Jia N, Jones R, Sukenick G, Patel D J. 2019. Second Messenger cA4 Formation within the Composite Csm1 Palm Pocket of Type III-A CRISPR-Cas Csm Complex and Its Release Path. Mol Cell 75: 933-943.e6. doi:10.1016/j.molcel.2019.06.013
Joung J, Ladha A, Saito M, Kim N-G, Woolley A E, Segel M, Barretto R P J, Ranu A, Macrae R K, Faure G, Ioannidi E I, Krajeski R N, Bruneau R, Huang M-L W, Yu X G, Li J Z, Walker B D, Hung D T, Greninger A L, Jerome K R, Gootenberg J S, Abudayyeh O O, Zhang F. 2020. Detection of SARS-CoV-2 with SHERLOCK One-Pot Testing. N Engl J Med 382:NEJMc2026172. doi:10.1056/NEJMc2026172
Katoh K, Standley D M. 2013. MAFFT Multiple Sequence Alignment Software Version 7: Improvements in Performance and Usability. Mol Biol Evol 30:772-780. doi:10.1093/molbev/mst010
Kazlauskiene M, Kostiuk G, Venclovas Č, Tamulaitis G, Siksnys V. 2017. A cyclic oligonucleotide signaling pathway in type III CRISPR-Cas systems. Science (80-) 357:605-609. doi:10.1126/science.aao0100
Kazlauskiene M, Tamulaitis G, Kostiuk G, Venclovas Č, Siksnys V. 2016. Spatiotemporal Control of Type III-A CRISPR-Cas Immunity: Coupling DNA Degradation with the Target RNA Recognition. Mol Cell 62:295-306. doi:10.1016/j.molcel.2016.03.024
La Scola B, Le Bideau M, Andreani J, Hoang V T, Grimaldier C, Colson P, Gautret P, Raoult D. 2020. Viral RNA load as determined by cell culture as a management tool for discharge of SARS-CoV-2 patients from infectious disease wards. Eur J Clin Microbiol Infect Dis 39:1059-1061. doi:10.1007/s10096-020-03913-9
Larremore D B, Wilder B, Lester E, Shehata S, Burke J M, Hay J A, Tambe M, Mina M J, Parker R. 2021. Test sensitivity is secondary to frequency and tuRNAround time for COVID-19 screening. Sci Adv 7:1-11. doi:10.1126/sciadv.abd5393
Lazer D, Santillana M, Perlis R H, Ognyanova K, Baum M A, Quintana A, Druckman J, Volpe J D, Chwe H, Simonson M. 2020. State of the Nation: A 50-State COVID-19 Survey: Report #8: Failing the Test: Waiting Times for COVID Diagnostic Tests Across the U.S. doi:10.31219/osf.io/gj9x8
Liu T Y, Iavarone A T, DouDNA J A. 2017. RNA and DNA targeting by a reconstituted Thermus thermophiles Type III-A CRISPR-Cas system. PLoS One 12:1-20. doi:10.1371/jouRNAl.pone.0170552
Liu T Y, Liu J J, Aditham A J, Nogales E, DouDNA J A. 2019. Target preference of Type III-A CRISPR-Cas complexes at the transcription bubble. Nat Commun 10. doi:10.1038/s41467-019-10780-2
Makarova K S, Timinskas A, Wolf Y I, Gussow A B, Siksnys V, Venclovas Č, Koonin E V. 2020. Evolutionary and functional classification of the CARF domain superfamily, key sensors in prokaryotic antivirus defense. Nucleic Acids Res 1-20. doi:10.1093/nar/gkaa635
Nasef M, Muffly M C, Beckman A B, Rowe S J, Walker F C, Hatoum-Aslan A, Dunkle J A. 2019. Regulation of cyclic oligoadenylate synthesis by the Staphylococcus epidermidis Cas10-Csm complex. Rna 25:948-962. doi:10.1261/RNA.070417.119
Nemudryi A, Nemudraia A, Wiegand T, Nichols J, Deann T, Hedges J F, Cicha C, Lee H, Vanderwood K K, Bimczok D, Jutila M, Wiedenheft B. 2021. SARS-CoV-2 genomic surveillance identifies naturally occurring truncations of ORF7a that limit immune suppression. medRxiv. doi:doi.org/10.1101/2021.02.22.21252253
Niewoehner O, Garcia-Doval C, Rostøl J T, Berk C, Schwede F, Bigler L, Hall J, Marraffini L A, Jinek M. 2017. Type III CRISPR-Cas systems produce cyclic oligoadenylate second messengers. Nature 548:543-548. doi:10.1038/nature23467
Notomi T, Okayama H, Masubuchi H, Yonekawa T, Watanabe K, Amino N, Hase T. 2000. Loop-mediated isothermal amplification of DNA. Nucleic Acids Res 28:E63. doi:10.1093/nar/28.12.e63
Paltiel A D, Zheng A, Walensky R P. 2020. Assessment of SARS-CoV-2 Screening Strategies to Permit the Safe Reopening of College Campuses in the United States. JAMA Netw open 3:e2016818. doi:10.1001/jamanetworkopen.2020.16818
Rambaut A, Holmes E C, O'Toole A, Hill V, McCrone J T, Ruis C, du Plessis L, Pybus O G. 2020. A dynamic nomenclature proposal for SARS-CoV-2 lineages to assist genomic epidemiology. Nat Microbiol 5:1403-1407. doi:10.1038/s41564-020-0770-5
Rolando J C, Jue E, Barlow J T, Ismagilov R F. 2020. Real-time kinetics and high-resolution melt curves in single-molecule digital LAMP to differentiate and study specific and non-specific amplification. Nucleic Acids Res 48:e42. doi:10.1093/nar/gkaa099
Rouillon C, Athukoralage J S, Graham S, Grüschow S, White M F. 2018. Control of cyclic oligoadenylate synthesis in a type III CRISPR system. Elife 7:1-22. doi:10.7554/eLife.36734
Samai P, Pyenson N, Jiang W, Goldberg G W, Hatoum-Aslan A, Marraffini L A. 2015. Co-transcriptional DNA and RNA cleavage during type III CRISPR-Cas immunity. Cell 161:1164-1174. doi:10.1016/j.cell.2015.04.027
Sofos N, Feng M, Stella S, Pape T, Fuglsang A, Lin J, Huang Q, Li Y, She Q, Montoya G. 2020. Structures of the Cmr-β Complex Reveal the Regulation of the Immunity Mechanism of Type III-B CRISPR-Cas. Mol Cell 79:741-757.e7. doi:10.1016/j.molcel.2020.07.008
Staals R H J, Zhu Y, Taylor D W, Kornfeld J E, Sharma K, Barendregt A, Koehorst J J, Vlot M, Neupane N, Varossieau K, Sakamoto K, Suzuki T, Dohmae N, Yokoyama S, Schaap P J, Urlaub H, Heck A J R, Nogales E, DouDNA J A, Shinkai A, vanderOost J. 2014. RNA Targeting by the Type III-A CRISPR-Cas Csm Complex of Thermus thermophilus. Mol Cell 56:518-530. doi:10.1016/j.molcel.2014.10.005
Tamulaitis G, Kazlauskiene M, Manakova E, Venclovas Č, Nwokeoji A O, Dickman M J, Horvath P, Siksnys V. 2014. Programmable RNA Shredding by the Type III-A CRISPR-Cas System of Streptococcus thermophilus. Mol Cell 56:506-517. doi:10.1016/j.molcel.2014.09.027
Tomita N, Mori Y, Kanda H, Notomi T. 2008. Loop-mediated isothermal amplification (LAMP) of gene sequences and simple visual detection of products. Nat Protoc 3:877-882. doi:10.1038/nprot.2008.57
Tyson J R, James P, Stoddart D, Sparks N, Wickenhagen A, Hall G, Choi J H, Lapointe H, Kamelian K, Smith A D, Prystajecky N, Goodfellow I, Wilson S J, Harrigan R, Snutch T P, Loman N J, Quick J. 2020. Improvements to the ARTIC multiplex PCR method for SARS-CoV-2 genome sequencing using nanopore. bioRxiv. doi:10.1101/2020.09.04.283077
Wölfe R, Corman V M, Guggemos W, Seilmaier M, Zange S, Müller M A, Niemeyer D, Jones T C, Vollmar P, Rothe C, Hoelscher M, Bleicker T, Brünink S, Schneider J, Ehmann R, Zwirglmaier K, Drosten C, Wendtner C. 2020. Virological assessment of hospitalized patients with COVID-2019. Nature 581:465-469. doi:10.1038/s41586-020-2196-x
Yan W X, Hunnewell P, Alfonse L E, Carte J M, Keston-Smith E, Sothiselvam S, Garrity A J, Chong S, Makarova K S, Koonin E V., Cheng D R, Scott D A. 2019. Functionally diverse type V CRISPR-Cas systems. Science (80-) 363:88-91. doi:10.1126/science.aav7271
Zhang J, Graham S, Tello A, Iu H, White M F. 2016. Multiple nucleic acid cleavage modes in divergent type III CRISPR systems. Nucleic Acids Res 44:1789-1799. doi:10.1093/nar/gkw020
Zhang Y, Ren G, Buss J, Barry A J, Patton G C, Tanner N A. 2020. Enhancing colorimetric loop-mediated isothermal amplification speed and sensitivity with guanidine chloride. Biotechniques 69:1-8. doi:10.2144/btn-2020-0078

All patent filings, websites, other publications, accession numbers and the like cited above or below are incorporated by reference in their entirety for all purposes to the same extent as if each individual item were specifically and individually indicated to be so incorporated by reference. If different versions of a sequence are associated with an accession number at different times, the version associated with the accession number at the effective filing date of this application is meant. The effective filing date means the earlier of the actual filing date or filing date of a priority application referring to the accession number if applicable. Likewise, if different versions of a publication, website or the like are published at different times, the version most recently published at the effective filing date of the application is meant unless otherwise indicated. Any feature, step, element, embodiment, or aspect of the disclosure can be used in combination with any other unless specifically indicated otherwise. Although the present disclosure has been described in some detail by way of illustration and example for purposes of clarity and understanding, it will be apparent that certain changes and modifications may be practiced within the scope of the appended claims.

What is claimed is:

1. A method of detecting nucleic acid in a sample, comprising:
contacting the sample with an engineered type III Clustered Regularly Interspaced Short Palindromic Repeat (CRISPR)-Cas system to detect NA in the sample, the engineered CRISPR-Cas system comprising:
a CRISPR guide comprising a CRISPR guide sequence that is complementary to a locus of the nucleic acid;
a first subunit that undergoes a conformational change upon binding of the engineered type III CRISPR-Cas system to the locus of the nucleic acid, the conformational change activating DNase activity of the first subunit and/or polymerase activity of the first subunit, the polymerase activity generating one or more products; and
detecting the DNase activity and/or the one or more products of the polymerase activity.

2. The method of claim 1, wherein the nucleic acid comprises a viral ribonucleic acid (RNA).

3. The method of claim 2, wherein the viral RNA comprises RNA of severe acute respiratory syndrome coronavirus 2 (SARS-CoV-2).

4. The method of claim 3, wherein the locus comprises a nucleocapsid gene (N-gene) of the SARS-CoV-2.

5. The method of claim 3, wherein the locus comprises a region of the viral RNA that is conserved among a plurality of SARS-CoV-2 genomes.

6. The method of claim 2, wherein the CRISPR guide sequence comprises a nucleic acid sequence of SEQ ID NO. 1.

7. The method of claim 2, wherein the CRISPR guide sequence comprises a nucleic acid sequence of SEQ ID NO. 2.

8. The method of claim 1, wherein the one or more products comprise a linear or cyclic oligonucleotide and wherein the detection system comprises instrumented fluorometric detection comprising:
an RNA tether linking a fluorophore to a quencher; and
a nuclease activated by the linear or cyclic oligonucleotide, the activated nuclease cleaving the RNA tether to thereby release the fluorophore that is detected by a fluorescence detecting instrument.

9. The method of claim 8, wherein the linear or cyclic oligonucleotide comprises a cyclic oligoadenylate, and wherein the nuclease activated by the linear or cyclic oligonucleotide comprises Csm6 or Can1.

10. The method of claim 8, wherein the instrumented fluorometric detection further comprises:
a deoxyribonucleic acid (DNA) tether linking the fluorophore or a second fluorophore to the quencher or a second quencher,
wherein the DNase activity cleaves the DNA tether to thereby release the fluorophore or the second fluorophore.

11. The method of claim 1, wherein the detection system comprises instrumented fluorometric detection comprising:
a deoxyribonucleic acid (DNA) tether linking a fluorophore to a quencher,
wherein the first subunit has a DNase activity that is activated upon hybridization of the RNA guide to the locus of the viral RNA, the DNase activity cleaving the DNA tether to thereby release the fluorophore that is detected.

12. The method of claim 1, wherein the one or more products comprise a linear or cyclic oligonucleotide and wherein the detection system comprises instrumented fluorometric detection comprising:
a deoxyribonucleic acid (DNA) tether linking a fluorophore to a quencher; and a nuclease activated by the linear or cyclic oligonucleotide, the activated nuclease cleaving the DNA tether to thereby release the fluorophore that is detected by a fluorescence detecting instrument.

13. The method of claim 1, wherein the one or more products comprise a pyrophosphate, and wherein the detection system comprises visible fluorometric detection comprising:
a fluorescent dye quenched by a quencher;
wherein the pyrophosphate forms an insoluble precipitate with the quencher to thereby unquench the fluorescent dye that is detected based on a color change.

14. The method of claim 13, wherein the fluorescent dye comprises calcein and the quencher comprises Manganese, and wherein unquenched calcein is bound by Magnesium to form a fluorescent complex that is detected.

15. The method of claim 1, wherein the one or more products comprise protons, and wherein the detection system comprises a colorimetric system, the colorimetric system comprising:
a solution comprising a pH-sensitive dye; and
wherein the protons acidify the solution, resulting in a change in color of the pH-sensitive dye.

16. The method of claim 1, wherein the engineered type III CRISPR-Cas system further comprises:
an engineered second subunit comprising a backbone subunit of the engineered type III CRISPR-Cas system with an introduced mutation, the engineered second subunit having RNase activity when in wildtype form, but the introduced mutation disrupting the RNase activity to prevent degradation of the viral RNA, thereby increasing signal generation of the detection system.

17. The method of claim 13, wherein the first subunit comprises a Cas10 subunit, the second subunit comprises Csm3, and wherein an activity of the Cas10 subunit is moderated by activity of the second subunit in the wildtype form, and wherein the introduced mutation to the second subunit disrupts the moderation of the Cas10 subunit.

18. The method of claim 16, wherein the wildtype form of the second subunit comprises an amino acid sequence of SEQ ID NO. 26 and the second subunit with the introduced mutation comprises an amino acid sequence of SEQ ID NO. 27.

19. The method of claim 1, wherein the one or more products comprise (i) a linear or cyclic oligonucleotide and (ii) protons, wherein the detection system comprises:
fluorometric detection comprising:
an RNA tether linking a fluorophore to a quencher;
a nuclease activated by the linear or cyclic oligonucleotide, the activated nuclease cleaving the RNA tether to thereby release the fluorophore that is detected; and
colorimetric detection comprising:
a solution comprising a pH-sensitive dye; and
wherein the solution is acidified by the protons resulting in a change in color of the pH-sensitive dye.

20. The method of claim 19, wherein the fluorometric detection further comprises:
a deoxyribonucleic acid (DNA) tether linking the fluorophore or a second fluorophore to the quencher or a second quencher,
wherein the DNase activity cleaves the DNA tether to thereby release the fluorophore or the second fluorophore.

21. The method of claim 1, wherein the one or more products comprise protons, wherein the detection system comprises:
fluorometric detection comprising:
a deoxyribonucleic acid (DNA) tether linking a fluorophore to a quencher, wherein the DNase activity cleaves the DNA tether to thereby release the fluorophore that is detected; and
colorimetric detection comprising:
a solution comprising a pH-sensitive dye; and
wherein the solution is acidified by the protons resulting in a change in color of the pH-sensitive dye.

22. The method of claim 1, wherein the nucleic acid comprises ribonucleic acid (RNA), the system further comprising: a reverse transcription loop-mediated isothermal amplification (RT-LAMP) primer having a T7 binding site for RT-LAMP-T7 amplification of the RNA.

23. The method of claim 22, wherein the RT-LAMP-T7 amplification and the detection of the RNA comprises a single pot combination.
